(19) United States
(12) Patent Application Publication
(10) Pub. No.: US 2004/0033595 A1
(43) Pub. Date: Feb. 19, 2004

(54) Conditionally replicating vectors for inhibiting viral infections

(76) Inventors: Laurent Humeau, Gaithersburg, MD (US); Yuexia Li, Gaithersburg, MD (US); Randall Merling, North Potomac, MD (US); Boro Dropulic, Ellicott City, MD (US); Kathy L. Schonely, Germantown, MD (US)

Correspondence Address: MORRISON & FOERSTER LLP, 3811 VALLEY CENTRE DRIVE, SUITE 500, SAN DIEGO, CA, 92130-2332, US

(21) Appl. No.: 10/388,667

(22) Filed: Mar. 14, 2003

Related U.S. Application Data
(63) Continuation of application No. 09/819,401, filed on Mar. 27, 2001.

(51) Int. Cl. C12N 15/63; C12N 15/09; C12N 15/00; C12N 15/74; C12N 15/70; A61K 39/21; C07H 21/04

(52) U.S. Cl. 435/320.1; 424/207.1; 536/23.72

(57) ABSTRACT

The present invention provides improved conditionally replicating vectors that have improved safety against the generation of replication competent vectors or virus. Also disclosed are methods of making, propagating and selectively packaging, modifying, and using such vectors. Included are improved helper constructs, host cells, for use with the improved vectors as well as pharmaceutical compositions and host cells comprising the vectors, the use of vector containing host cells to screen drugs, and methods of using the vectors to determine gene function. The methods also include the prophylactic and therapeutic treatment of disease, especially viral infection, and HIV infection in particular.

### RELATED APPLICATIONS

This application is related to U.S. patent application Ser No. 09/524,006, filed Mar. 13, 2000, and Ser. No. 09/667,893, filed Sep. 22, 2000, which are hereby incorporated by reference as if fully set forth.

### TECHNICAL FIELD OF THE INVENTION

The present invention provides improved conditionally replicating vectors and methods for their use in the prophylactic and therapeutic treatment of viral infections, especially viral infection, and HIV infection in particular, as well as diseases associated therewith.

### BACKGROUND OF THE INVENTION

The discovery of the human immunodeficiency virus (HIV), a lentivirus, as the cause of acquired immune deficiency syndrome (AIDS) has fostered a plethora of research into the underlying mechanisms of the viral infectious cycle and viral pathogenesis. Studies on these mechanisms have provided researchers with an ever-increasing number of targets for the development of antiviral agents effective not only against HIV, but against HIV products, its genome, and other viruses as well. These antiviral agents, particularly those directed against HIV, can be categorized into groups depending on their mode of action. Such groups include inhibitors of reverse transcriptase, competitors of viral entry into cells, vaccines, and protease inhibitors, as well as a more recent group referred to herein as “genetic antiviral agents.” Generally, each type of antiviral agent has its own associated benefits and limitations, and must be assessed in terms of the exigencies of the particular treatment situation. Antiviral agents, such as zidovudine (3′-azido-3′-deoxythymidine, also known as AZT), protease inhibitors and the like, can be delivered into the cells of a patient's body with relative ease and have been studied extensively. Targeting one specific factor in the viral infectious cycle, such agents have proven relatively ineffective against HIV. This is primarily due to the fact that strains of HIV change rapidly and become resistant to agents having a singular locus of effect (Richman, AIDS Res. and Hum. Retrovir., 8, 1065-1071 (1992)). Accordingly, the problems of genetic variation and rapid mutation in HIV genomes compel consideration of new antiviral strategies to treat HIV infections. Along these lines, genetic antiviral agents are attractive, since they work at many different levels intracellularly. Genetic antiviral agents differ from other therapeutic agents in that they are transferred as molecular elements into a target cell, wherein they protect the cell from viral infection (Baltimore, Nature, 325, 395-396 (1988); and Dropulic' et al., Hum. Gene Ther., 5, 927-939 (1994)). Genetic antiviral agents can be any genetic sequence and include, but are not limited to, antisense molecules, RNA decoys, transdominant mutants, interferons, toxins, immunogens, and ribozymes. In particular, ribozymes are antisense-like genetic antiviral agents that cleave target RNAs, including HIV RNA, in a sequence-specific fashion. The specificity of ribozyme-mediated cleavage of target RNA suggests the possible use of ribozymes as therapeutic inhibitors of viral replication, including HIV replication. Different types of ribozymes, such as the hammerhead and hairpin ribozymes, have been used in anti-HIV strategies (see, e.g., U.S. Pat. Nos. 5,144,019, 5,180,818 and 5,272,262, and PCT patent application nos. WO 94/01549 and WO 93/23569). Both of the hammerhead and hairpin ribozymes can be engineered to cleave any target RNA that contains a GUC sequence (Haseloff et al., Nature, 334, 585-591 (1988); Ulhlenbeck, Nature, 334, 585 (1987); Hampel et al., Nuc. Acids Res., 18, 299-304 (1990); and Symons, Ann. Rev. Biochem., 61, 641-671 (1992)). Generally speaking, hammerhead ribozymes have two types of functional domains, a conserved catalytic domain flanked by two hybridization domains. The hybridization domains bind to sequences surrounding the GUC sequence and the catalytic domain cleaves the RNA target 3′ to the GUC sequence (Uhlenbeck (1987), supra; Haseloff et al. (1988), supra; and Symons (1992), supra).

A number of studies have confirmed that ribozymes can be at least partially effective at inhibiting the propagation of HIV in tissue culture cells (see, e.g., Sarver et al., Science, 247, 1222-1225 (1990); Sarver et al., NIH Res., 5, 63-67 (1993a); Dropulic' et al., J. Virol., 66, 14321441 (1992); Dropulic' et al., Methods: Comp. Meth. Enzymol., 5, 43-49 (1993); Ojwang et al., PNAS, 89, 10802-10806 (1992); Yu et al., PNAS, 90, 6340-6344 (1993); and Weerasinghe et al., J. Virol., 65, 5531-5534 (1991)). In particular, Sarver et al. ((1990), supra) have demonstrated that hammerhead ribozymes designed to cleave within the transcribed region of the HIV gag gene, i.e., anti-gag ribozymes, could specifically cleave HIV gag RNAs in vitro. Furthermore, when cell lines expressing anti-gag ribozymes were challenged with HIV-1, a 50- to 100-fold inhibition of HIV replication was observed. Similarly, Weerasinghe et al. ((1991), supra) have shown that retroviral vectors encoding ribozymes designed to cleave within the U5 sequence of HIV-1 RNA confer HIV resistance to transduced cells upon subsequent challenge with HIV. Although different clones of transduced cells demonstrated different levels of resistance to challenge as determined by the promoter system used to drive ribozyme expression, most of the ribozyme-expressing cell lines succumbed to HIV expression after a limited time in culture.

Transduction of tissue culture cells with a provirus into the nef gene (which is not essential for viral replication in tissue culture) of which was introduced a ribozyme, the hybridization domains of which Were specific for the U5 region of HIV, has been shown to inhibit viral replication within the transduced cells 100-fold as compared to cells transduced with wild-type proviruses (see, e.g., Dropulic' et al. (1992) and (1993), supra). Similarly, hairpin ribozymes have been shown to inhibit HIV replication in T-cells transduced with vectors containing U5 hairpin ribozymes and challenged with HIV (Ojwang et al. (1992), supra). Other studies have shown that vectors containing ribozymes expressed from a tRNA promoter also inhibit a variety of HIV strains (Yu et al. (1993), supra).

Delivery of ribozymes or other genetic antiviral agents to the cellular targets of HIV infection (e.g., CD4+ T-cells and monocytic macrophages) has been a major hurdle for effective genetic therapeutic treatment of AIDS. Current approaches for targeting cells of the hematopoietic system (i.e., the primary targets for HIV infection) call for introduction of therapeutic genes into precursor multipotent stem cells, which, upon differentiation, give rise to mature T-cells, or, alternatively, into the mature CD4+ T lymphocytes, themselves. The targeting of stem cells is problematic, however, since the cells are difficult to culture and transduce in vitro. The targeting of circulating T lymphocytes is also problematic, since these cells are so widely disseminated that it is difficult to reach all target cells using current vector delivery systems. Moreover, macrophages need to be considered as a cellular target, since they are the major reservoir for viral spread to other organs. However, since macrophages are terminally differentiated and, therefore, do not undergo cellular division, they are not readily transduced with commonly used vectors. Accordingly, the predominant current approach to HIV treatment makes use of replication-defective viral vectors and packaging (i.e., “helper”) cell lines (see, e.g., Buchschacher, JAMA, 269(22), 2880-2886 (1993); Anderson, Science, 256, 808-813 (1992); Miller, Nature, 357, 455-460 (1992); Mulligan, Science, 260, 926-931 (1993); Friedmann, Science, 244, 1275-1281 (1989); and Cournoyer et al., Ann. Rev. Immunol., 11, 297-329 (1993)) to introduce into cells susceptible to viral infection (such as HIV infection) a foreign gene that specifically interferes with viral replication, or that causes the death of an infected cell (reviewed by Buchschacher (1993), supra). Such replication-defective viral vectors contain, in addition to the foreign gene of interest, the cis-acting sequences necessary for viral replication but not sequences that encode essential viral proteins. Consequently, such a vector is unable to complete the viral replicative cycle, and a helper cell line, which contains and constitutively expresses viral genes within its genome, is employed to propagate it. Following introduction of a replication-defective viral vector into a helper cell line, proteins required for viral particle formation are provided to the vector in trans, and vector viral particles capable of infecting target cells and expressing therein the gene, which interferes with viral replication or causes a virally infected cell to die, are produced.

Such replication-defective retroviral vectors include adenoviruses and adeno-associated viruses, as well as those retroviral vectors employed in clinical trials of HIV gene therapy, and, in particular, the mouse amphotropic retroviral vector known as the Moloney murine leukemia virus (MuLV). These defective viral vectors have been used to transduce CD4+ cells with genetic antiviral agents, such as anti-HIV ribozymes, with varying degrees of success (Sarver et al. (1990), supra; Weerasinghe et al. (1991), supra; Dropulic' et al. (1993), supra; Ojwang et al. (1992), supra; and Yu et al. (1993), supra). However, these vectors are intrinsically limited for HIV gene therapy applications. For example, a high transduction frequency is especially important in the treatment of HIV, where the vector has to transduce either rare CD34+ progenitor hematopoietic stem cells or widely disseminated target CD4+ T-cells, most of which, during the clinical “latent” stage of disease, are already infected with HIV. MuLV vectors, however, are difficult to obtain in high titer and, therefore, result in poor transduction. Furthermore, long-term expression of transduced DNA has not been obtained in CD34+ progenitor stem cells, in particular after differentiation to mature T lymphocytes. In addition, the use of defective viral vectors requires ex vivo gene transfer strategies (see, e.g., U.S. Pat. No. 5,399,346), which can be expensive and beyond the cost of the general population.

These shortcomings associated with the use of currently available vectors for genetic therapeutic treatment of AIDS have led researchers to seek out new viral vectors. One such vector is HIV, itself. HIV vectors have been employed for infectivity studies (Page et al., J. Virol., 64, 52705276 (1990)) and for the introduction of genes (such as suicide genes) into CD4+ cells, particularly CD4+ HIV-infected cells (see, e.g., Buchschacher et al., Hum. Gener. Ther., 3, 391397 (1992); Richardson et al., J. Virol., 67, 3997-4005 (1993); Carroll et al., J. Virol, 68, 60476051 (1994); and Parolin et al., J. Virol., 68, 3888-3895 (1994)). The strategy of these studies is to use HIV vectors to introduce genes into the CD4+ T-cells and monocytic cells. To date, however, these vectors are extremely complex. Moreover, use of these vectors is accompanied by a risk of generating wild-type HIV via intracellular recombination. Cotransfection/coinfection of defective vector sequences and helper virus has been observed to result in recombination between homologous regions of the viral genomes (Inoue et al., PNAS, 88, 2278-282 (1991)). Observed complementation in vitro indicates that a similar replication-defective HIV vector could recombine in vivo, thus exacerbating an already existing HIV infection. The fact that retroviruses package two RNA genomes into one virion has led researchers to suggest that retroviruses carry two viral RNAs to circumvent any genetic defects caused by complementation and/or recombination (Inoue et al. (1991), supra).

In addition to the risk of intracellular recombination, thereby resulting in wild-type HIV, HIV vectors have an associated risk of mutation in vivo, which increases the pathogenicity of the viral vector. This has lead Sarver et al. (AIDS Res. and Hum. Retrovir., 9, 483-487 (1993b)) to speculate regarding the development of second-generation recombinant HIV vectors, which are replication-competent, yet nonpathogenic. Such vectors, in comparison with the predominantly used nonreplicating vectors (i.e., replication-deficient vectors) continue to replicate in a patient, thus providing constant competition with wild-type HIV. So far, however, such vectors are not available.

Ideally, the best opportunity to treat an infected individual occurs at the time of inoculation, before the virus even infects the host. However, this is difficult to accomplish inasmuch as many individuals do not realize they have become infected with HIV until the clinical latent phase of disease. Based on this, the stage at which antiviral intervention is most sorely needed is during clinical latency. Therapy at this stage requires that the challenge presented by the large number of already infected CD4+ lymphocytes, which harbor viral genomes, be confronted. This is no trivial challenge, as evidenced by the fact that, to date, HIV remains incurable and is only poorly treatable by currently available therapies. An effective vaccine is not forthcoming, and, although inhibitors of reverse transcriptase and protease have been shown to prevent HIV replication in tissue culture, the development of viral resistance in vivo has led to treatment failure. Thus, HIV gene therapy may have little benefit for the vast majority of HIV-infected individuals, which to date is over 30 million.

In view of the above, it is also becoming increasingly important to develop long-term and persistent immunological responses to certain pathogens, especially viruses, particularly in the context of AIDS and cancer, for example. Live-attenuated (LA) vaccines, using replication-competent, but nonpathogenic viruses have been considered (Daniel et al., Science, 258, 1938-1941 (1992); and Desrosiers, AIDS Res. & Human Retrovir., 10, 331-332 (1994)). However, such nonpathogenic viruses, which differ from the corresponding wild-type viruses by a deletion in one or more genes, either (i) cannot elicit a protective immune response because the antigen does not persist (because the LA-virus does not efficiently replicate); or (ii) the LA-virus replicates but has other pathogenic potential, as witnessed by the ability of the LA-virus to cause disease in young animal models (Baba et al., Science, 267, 1823-1825 (1995)).

For the aforementioned reasons, there remains a need for alternative prophylactic and therapeutic treatment modalities of viral infection, particularly in the context of AIDS and cancer. The present invention provides such alternative methods by providing a conditionally replicating vector. The invention also provides additional methods in which such a vector can be employed. Further provided by the invention are helper vector constructs which complement the conditionally replicating vector to permit its replication and packaging as viral particles and virions. Such helper vectors are modified such that recombination with the conditionally replicated vector is minimized. Various embodiments of such helper-vector constructs are described. These and other objects and advantages of the present invention, as well as additional inventive features, will be apparent from the description of the invention set forth herein.

### BRIEF SUMMARY OF THE INVENTION

The present invention provides improved conditionally replicating vectors as well as improved compositions and methods for the production and use of said vectors. A conditionally replicating vector is characterized by a capacity to replicate only in a host cell that is permissive for replication of the vector. The improved vectors provided hereby have increased safety by being at reduced risk of regaining replication competency. As such, the vectors may also be referred to as “reduced recombination” vectors.

In one aspect of the invention, an improved conditionally replicating vector comprises at least one nucleic acid sequence, the presence, transcription or translation of which confers to the vector in a replication-permissive host cell a selective advantage over a wild-type strain of virus corresponding to the virus from which the vector was derived. Preferably, the improved conditionally replicating vector comprises at least one nucleic acid sequence which confers to the vector a selective advantage for replication over any other competing genome or genomic element. A genomic element as used herein is defined as a nucleic acid sequence in a host cell that is not derived from either the vector or any wild-type virus and that can compete, interfere or affect vector replication in the host cell. A genomic element is also not an entire genome, such as that of the host cell or another vector or virus. The selective advantage for replication is conferred by the presence, transcription, or translation of said nucleic acid sequence.

In another preferred embodiment, the conditionally replicating vector is a retrovirus, especially a lentivirus, and comprises at least one nucleic acid sequence, the presence, transcription or translation of which confers to a host cell, which is infected with the vector, a selective advantage over a cell infected with a wild-type strain of virus corresponding to the virus from which the vector was derived. Alternatively, the vector is not wholly derived from the wild-type strain, but is chimeric, containing components derived from more than one wild-type virus. The more than one wild-type of virus may be different (or heterologous) viruses or different strains or isolates of one virus. Preferably, said nucleic acid sequence may be expressed to provide a prophylactic effect in said host cell. This results in the cell being conferred with a survival advantage since the nucleic acid sequence prevents any infecting virus from replicating to levels that cause cell death.

Another preferred embodiment is an improved conditionally replicating plasmid vector comprising at least one nucleic acid sequence which confers to the vector a selective advantage for replication over any other competing vector or plasmid molecule. For example, such vectors may comprise a sequence (such as for example, a ribozyme or an antisense sequence) capable of cleaving or destroying, or resulting in the cleavage or destruction of, resulting in the cleavage of a competing vector or plasmid when they are colocalized with the vector. If the competing vector and the helper are to be destroyed, then the vector of the invention is engineered to contain other sequences to provide it with an overall selective advantage for replication. For example, the vector of the invention may contain an antisense sequence which destroys both the competing vector and the helper, yet the vector would have a selective advantage for replication because it additionally contains a second first-nucleotide sequence (e.g. a promoter that produces more vector RNA over competing vector RNA, the copy number of the vector in the transduced cell is higher than the competing genome, or a packaging signal that is present in the vector but not in a helper) that provides the conditionally replicating vector with the overall selective advantage. Therefore, the at least first nucleotide sequence further provides an overall selective advantage replication of the invention's vectors in this embodiment because the two first nucleotide sequences (i.e. the first first-nucleotide sequence being targeting the competing vector and/or the helper and the second first-nucleotide sequence provides a selective advantage for replication) work in concert to achieve the overall selective advantage effect. Therefore, synergistic effects of multiple first-nucleotide sequences can lead to profound selection conditions for the conditionally replicating vector, where any individual first-nucleotide sequence provides discriminatory selection over the competing genome.

Cleavage or destruction of the competing vector or plasmid also diminishes the chance of full length recombination with the vector, which thus has the “reduced recombination” property. The vector can be protected from cleavage by degenerating its sequence to not be targeted by a ribozyme or antisense sequence. The vector can be further protected from cleavage by engineering the vector, or the genomic version of the vector, to differentially track the RNA so that genomic vector RNA is not significantly cleaved by the competing vector or helper. Alternatively, the helper could be similarly and solely engineered by, for example, inserting splicing elements to force the helper into the spliceosome, while engineering the vector genomic RNA to contain first nucleic acid sequences that are present only in unspliced but not spliced vector RNA. Such non-limiting examples of improved vectors are particularly advantageous when used during production of the a viral vector or a plasmid vector and the cloning or subcloning of nucleic acid sequences into such vectors, as well as their use in mutagenesis, inducible gene expression and the like.

Also provided by the present invention is a pharmaceutical composition comprising a conditionally replicating vector of the invention and a pharmaceutically acceptable carrier. Further provided is a host cell comprising a conditionally replicating viral vector. A vector, wherein said vector, if DNA, comprises a nucleotide sequence selected from the group consisting of SEQ ID NOS:2, 3, 4, 5, 6, 14, in which at least one N is mutated, 15 and 16 and wherein said vector, if RNA, comprises a nucleotide sequence encoded by a nucleotide sequence selected from the group consisting of SEQ ID NOS:2, 3, 4, 5, 6, 14, in which at least one N is mutated, 15 and 16 is also provided as are isolated and purified nucleic acid molecules as set forth herein. Similarly provided are a method of engendering a vector with a ribozyme, a method of modifying a vector, and a method of propagating and selectively packaging a conditionally replicating vector without using a packaging cell line.

In yet another aspect of the present invention, a method of therapeutically and prophylactically treating a host cell for a viral infection is provided. In particularly preferred embodiments, such treatment is effected in said host by conferring of a dominant phenotype that inhibits viral infection or by conferring a phenotype that inhibits infection by other viruses. Preferably, the other viruses inhibited include a broad range of viral strains. Such methods can additionally comprise the use of a helper-expression vector, also referred to as a “helper vector” or “helper vector construct,” a cytotoxic drug, proteins/factors, or a protease/reverse transcriptase inhibitor as appropriate. The method can be used, for example, to inhibit replication of a virus, to treat diseases (including cancer, genetic, infectious, vascular & other diseases), to conduct efficient ex vivo and in vivo gene transfer, to permit safe vector production, to determine the function of a gene, or to express a gene of interest in a host cell.

In yet another aspect of the invention, a method of using a host cell comprising a conditionally replicating vector of the invention to detect interaction between a drug/factor and a protein is provided. Such a method enables protein characterization and screening of drugs, factors, or other proteins for activity or physical interactions with respect to a given protein encoded by the vector. The method further permits the identification and characterization of proteins that are functionally related to a given protein encoded by the vector. For example, if the encoded protein is a transcription factor, its expression by a vector of the invention permits the identification of genes regulated by the transcription factor.

The invention also provides compositions and conditions for the storage of vectors under various conditions prior to their use. Examples of such conditions include storage at −80° C., 20° C., and 4° C. in the presence of various carriers.

Further embodiments of the present invention include generic lentiviral vectors, modifications of a helper-vector construct that serve to diminish, minimize, or eliminate recombination of the helper-vector with the conditionally replicating vector to generate a replication competent vector or virus (RCV). Hence the invention includes helper-vectors that serve to prepare “reduced recombination” vectors. A helper vector of the invention also preferably increases the titer of the produced conditionally replicating vector to levels greater than 10⁷ transducing units per milliliter. Other embodiments include concentrating a vector using high-speed (but not ultra) centrifugation and chromatographic, ultrafiltration, and diafiltration methods for concentration and purification.

Helper vector modifications of the invention include the insertion of a ribozyme, such as an anti-U5 ribozyme, or an antisense sequence that cleaves or results in the destruction or inactivation of the conditionally replicating vector in the event the helper-vector and conditionally replicating vector are co-localized or co-packaged. In one embodiment, the helper components are integrated entirely on one plasmid construct to create a two plasmid functional vector-helper system that efficiently produces unconcentrated vector supernatant titers of greater than 10⁷ transducing units per ml (see FIG. 3 herein). In another embodiment, the nucleotide sequence of the helper-vector is degenerated to minimize recombination with the conditionally replicating vector. In yet another embodiment, the helper-vector comprises heterologous transacting elements for use in packaging the conditionally replicating vector. Examples of such elements include, but are not limited to, the vesicular stomatis virus envelope protein VSV-G, the RD114 envelope protein, the Rabies Virus envelope protein, the Gibbon Ape Leukemia Virus envelope protein (GALV) and chimeric envelope proteins. Further modifications also include heterologous rev-responsive elements (RREs), post-transcriptional regulatory elements (PRE) or constitutive transport elements (CTEs). In still another embodiment, the helper-vector construct further comprises splice donor, splice acceptor sites, or sites for degenerated or humanized nucleotide sequences. For example, the splice sites can be located such that the packaging signal and/or RRE may be removed from transcribed RNAs by a splicing event. Furthermore, sites for intron insertion into vector or helper constructs are provided such that dimerization, co-localization, or recombination between the conditionally replicating vector and helper, or other competing genome or genomic element, is minimized. The insertion of introns may be directed to result in the trafficking of helper RNA into spliceosomes and away from the vector RNA. Surprisingly, some helper vector constructs were found to increase the titer of the conditionally replicating vector.

### BRIEF DESCRIPTION OF THE FIGURES

FIGS. 1A-1K are schematic depictions of specific improved conditionally replicating vectors encompassed by the present invention: pN1(cPT), pN1(cPTc)ASenvGFP(464), pN1(cPT)ASenvGFP(452), pN1(cPT2)ASenvGFP, pN1 (cPT)cGFP, pN1 GFP(cPT)T, pN1(cPT)GFPTAR, pN1GFP(cPT)VT, pN2GFP, pN2ASenvGFP(418), and pN2(spe)ASenvGFP. Of course the marker gene for green fluorescent protein (GFP) may be removed before use of the vectors in applications described herein. Designations: N1, minimal HIV-1 derived vector without gag/pol sequences but with packaging sequence from gag indicated as gag′ or gag″ and a termination codon placed approximately 40 basepairs from the ATG of the gag sequence; N2, HIV-1 derived vector capable of expressing gag/pol sequences; AS, antisense; ASenv, env sequence present in antisense orientation; gag, pol and env, the coding sequence for proteins that form the viral core, reverse transcriptase, and envelope, respectively; tat, rev, rre, and nef, additional viral genes; cPTc, minimal central polypyrimidine tract sequence; cPT, central polypyrimidine tract containing large insert of about 548 basepairs; cPT2, central polypyrimidine tract containing sequence of about 438 basepairs (does not include gag sequences as much as cPT); spe, gag/pol sequences are not translated; GFP, green fluorescent protein encoded.

FIG. 2 depicts the DNA sequences of wild-type HIV U5 RNA SEQ ID NO:1 (A) and modified crHIV U5 RNA SEQ ID NO:2 (B). Numbers refer to the number of bases downstream from the start of transcription.

FIGS. 3A-3E illustrate effects of the helper vector to conditionally replicating (cr) vector ratio on the titer of cr vectors produced. FIG. 3A shows the molar ratio of 1:0.5 to result in the highest titer for pN1(cPTc)GFP; 3B and 3C show the molar ratio of 1:0.75 to provide the highest titer for pN1(cPT)GFP and pN1(cPT2)ASenvGFP, respectively; and 3D and 3E show the molar ratio of 1:1.5 to provide the highest titer for pN1cGFP and pN2cGFP, respectively. The vectors are shown in FIG. 1 except for pN1(cPT)GFP, which lacks the antisense env sequence and for pN1cGFP and pN2cGFP, which have an inserted cytomegalovirus (CMV) promoter to express GFP. The figures indicate that conditions for the production of titers of at least 1.5×10⁷ transducing units per ml have been achieved with a two plasmid system.

FIGS. 4A and 4B show maps of two HIV-2 based conditionally replicating vectors: pS1 cGFP and pS2cGFP. The designations pS1 and pS2 refer to the absence or presence of the gag/pol sequences as described above for pN1 and pN2. The designation c indicates the presence of a CMV promoter to direct expression of GFP.

FIGS. 5A and 5B illustrate the effects of different helper vector constructs on the packaging of pS1cGFP and pS2cGFP. The effect of different molar ratios between helper vector and conditionally replicating vector were tested along with the effect of different RREs on the helper vector. As indicated, the use of a 1:1 ratio of helper vector to conditionally replicating vector was more efficient at producing functional vector particles than the other ratios tested.

FIGS. 6A-6G depict the structures of various helper vector constructs: pVIRPAC-1, pVIRPAC-2, pVIRPAC-1.1Rz, pVIRPAC-1.2, VirPac1.2Rz, pVIRPAC-1.2Rz2, and pVIRPAC 1.2RzIn as encompassed by the present invention. Rz refers to the presence of an anti-U5 ribozyme while 1.1 and 1.2 refer to the helper having an RRE derived from HIV-1 and HIV-2, respectively.

FIG. 7 illustrates the influence of one or more ribozymes on the titers of viral vector. pN1(cPT)GFP was packaged in HeLa-tat cells in the presence of pVirPac1.2 (containing no ribozymes), pVirPac1.2RzIn (containing one ribozyme and an intron), pVP1.2Rz (containing one ribozyme) or pVP1.2Rz2 (containing two ribozymes). As shown by the graph, the presence of one ribozyme (pVirPac1.2Rz) or a ribozyme and an intron designed to affect cellular trafficking of helper RNA (pVirPac1.2RzIn) had no significant effect on vector titer. PCR analysis of titer samples for co-packaged helper constructs indicated low co-packaging in the presence of a ribozyme versus high co-packaging in the absence of a ribozyme. The indicator “VirPac” may also be denoted “VIRPAC” or “VP”.

FIG. 8 illustrates the inhibitory effect of vectors from the pN1 and pN2 series on wild-type HIV replication in T cells. T cells were first transduced with the vector and then challenged with wild-type virus at a multiplicity of infection of 0.1 with 100% of the cells transduced. The replication of virus was assayed by p24 ELISA antigen capture assay. pN2ASenvGFP demonstrated a profound inhibition of wild-type virus replication.

FIGS. 9A and 9B show the potent inhibition of wild-type HIV replication by pN1 and pN2 based vectors. FIG. 9A shows the potent inhibition of wild-type HIV in human T cells by pN1GFP(cPT)VT and pN2ASenvGFP in comparison to control tumor cells upon infection by wild-type HIV. FIG. 9B shows similar results in T cells with pN1(cPT)ASenvGFP and pN2ASenvGFP.

FIGS. 10A and 10B shows vectors and their use to select transduced cells. FIG. 10A shows the organization of the vectors used, where pN1CMIG and pN1MCG contain an internal CMV promoter while pN1MIG and pN1MIG-W express the MGMT gene via the HIV-LTR promoter.

FIG. 10B shows a graph of the expansion of SupT1 cells transduced with the above vectors and selected with BG and BCNU as described in Example 5 below.

FIG. 11, panels A-F, show the selection of transduced primary CD4+ cells with BG and BCNU. CD4+ cells transduced with pN2MIG were cultured in 0, 0.5, 2, 5, 10, and 10 μM BG (shown in panels A-F, respectively) with the indicated concentrations of BCNU. Panel F further shows the results of diluting the transduced cells 1:5 followed by treatment with 10 μM BG and the indicated BCNU concentrations. The starting level of 3% GFP+cells in panel F were increased at least 32 fold to 97%, a level not previously seen in vitro.

FIG. 12 shows the effects of ribozymes on preventing co-packaging of the helper RNA into vector preparations. Virus containing supernatants were extracted by “boom extraction” and subjected to DNase I digestion followed by qualitative reverse transcriptase-PCR (RT-PCR) detection of the HIV-1 gag-pol region found in the helper construct. As shown, the presence of one or two ribozymes in the helper construct used (pVP1.2Rz orpVP1.2Rz2) significantly reduced the amount of copackaged helper vector over a ribozyme minus helper (pVP1.2).

FIGS. 13A and 13B show the ability to purge tumor cells from CD34+ stem cells. FIG. 13A shows that at an MOI of 10, pN1(cPT)ASenvGFP transduced 98.52% of SupT1 tumor cells after a single round of transduction. FIG. 13B shows that in transducing CD34+ cells, no significant transduction was seen after a single round of transduction. Only after three rounds was significant transduction observed. Designations: 1/2/A, for example, refers to 1 round of transduction, MOI of 2, and the presence of viral accessory proteins on the transduced vector while 3/50, for example, refers to three rounds of transduction and an MOI of 50.

FIG. 14A shows the structures of VSV-G wildtype, RD114 wildtype, and chimeric envelope proteins with the extracellular, transmembrane, and cytoplasmic domains indicated. FIG. 14B shows the titers of HIV-1 vectors pseudotyped in HT1080 with different envelope proteins (VSV-G wildtype, rabies virus G, RD114 wildtype, and RD114E, a chimeric VSV-G and RD114 construct).

FIGS. 15A-15E are schematic depictions of specific packaging line constructs encompassed by the present invention: p(CGCRSRRE), p(TREtTApuro), p(BI-RevTat), p(EH-GP), and p(CMVGP). FIG. 15F shows the organization of rev dependent VSV-G constructs.

FIG. 16 shows the yield of pN1(cPT)GFP vectors per cell factory before and after concentration in HeLa-tat cells. As shown, the yield remains at approximately 10¹⁰ under either set of conditions.

FIG. 17 shows the results from using a Rev/RRE/CRS system to control VSV-G expression.

FIG. 18 shows the influence of storage buffer on vector recovery after storage for 3-5 weeks at different temperatures. As shown, the presence of 10% trehalose or 10% glucose, in either D-PBS or HBS provided good recoveries of vector after storage at either −20 or −80° C. FIG. 19 shows the inhibition of wildtype HIV-1 DNA in cells containing vectors of the invention.

### DESCRIPTION OF THE PREFERRED EMBODIMENTS

The present invention provides improved conditionally replicating vectors, lentiviral vectors, and their use. In addition to being selectively replicated, the vectors contain specific modifications to reduce the likelihood of recombination to result in the vectors becoming replication competent. Included are methods of inhibiting the replication of a wild-type strain of a virus and methods for gene delivery into cells comprising such improved vectors. The method comprises contacting a host, which is capable of being infected, at risk of being infected, or preferably actually infected with such a wild-type strain of virus, with an improved vector that is propagated only in a host that is permissive for the replication of the vector (i.e., a nonpathogenic, or non-disease producing, conditionally replicating (cr) vector).

As further described herein, a particular aim of the method is to establish a competitive infection in the host with such a nonpathogenic, conditionally replicating vector. Generally, a conditionally replicating vector according to the invention comprises at least one nucleic acid sequence that confers a selective advantage for replication and spread to the conditionally replicating vector as compared with a wild-type virus, and/or at least one nucleic acid sequence that confers a selective advantage for propagation of viral particles to a host cell containing a conditionally replicating vector as compared with a host cell containing a wild-type virus.

In a preferred embodiment of the invention, the vector comprises an HIV sequence and is employed for treatment of HIV infection. Thus, the vector, or a host cell containing the vector, comprises at least one nucleic acid sequence that (1) provides a crHIV genome with a selective advantage over a wild-type HIV genome for packaging into progeny virions (i.e., in cells where they both reside), and/or (2) provides a host cell producing a conditionally replicating vector (virus) with a selective advantage for production of a crHIV virion, as compared with a host cell producing a wild-type virus. One method (to which the invention is not limited) is to confer crHIV genomes with a selective advantage for packaging by providing them with one or more ribozymes capable of cleaving the wild-type HIV genome.

In another aspect of the invention, the vectors are conferred with a reduced likelihood of recombination with wild-type and helper constructs to result in replication competent vectors. Additionally, helper vectors, cells, and packaging systems which also contribute to the reduction in recombination are provided to further reduce the chances of a productive recombination event to render the vectors no longer conditionally replicating.

Wild-Type Virus

According to the invention, a “virus” is an infectious agent that consists of protein and nucleic acid, and that uses a host cell's genetic machinery to produce viral products specified by the viral nucleic acid. A “nucleic acid” refers to a polymer of DNA or RNA that is single or double-stranded, linear or circular, and, optionally, contains synthetic, normatural, or modified nucleotides, which are capable of being incorporated into DNA or RNA polymers. A DNA polynucleotide preferably is comprised of genomic or cDNA sequences.

A “wild-type strain of a virus” is a strain that does not comprise any of the human-made mutations as described herein, i.e., any virus that can be isolated from nature. Alternatively, a wild-type strain is any virus that has been cultured in a laboratory, but still, in the absence of any other virus, is capable of producing progeny genomes or virions like those isolated from nature. For example, the pNL4-3 HIV-1 molecular clone described in the following Examples is a wildtype strain which is available from the AIDS Research and Reference Reagent Program Catalog through the National Institutes of Health (see, also, Adachi et al., J. Virol., 59, 284-291 (1986)). pPSXB is a HIV-2 molecular clone which was kindly obtained from Dr Suresh Arya at the National Institutes of Health, Bethesda, Md. and was described in Arya et al. Human immunodeficiency virus type 2 lentivirus vectors for gene transfer: expression and potential for helper virus-free packaging. Hum Gene Ther. Jun. 10, 1998;9(9):1371-80.

In general, the method of the present invention preferably is employed to treat viral diseases that result from viral infection. Desirably, a virus (as well as the vector, as discussed below) is an RNA virus, but also can be a DNA virus. RNA viruses are a diverse group that infects prokaryotes (e.g., the bacteriophages) as well as many eukaryotes, including mammals and, particularly, humans. Most RNA viruses have single-stranded RNA as their genetic material, although at least one family has double-stranded RNA as the genetic material. The RNA viruses are divided into three main groups: the positive-stranded viruses (i.e., those of which the genome transferred by the virus is translated into protein, and whose deproteinized nucleic acid is sufficient to initiate infection), the negative-stranded viruses (i.e., those of which the genome transferred by the virus is complementary to the message sense, and must be transcribed by virion-associated enzymes before translation can occur), and the double-stranded RNA viruses. The method of the present invention preferably is employed to treat positive-stranded viruses, negative-stranded viruses, and double-stranded RNA viruses.

As employed herein, an RNA virus encompasses Sindbis-like viruses (e.g., Togaviridae, Bromovirus, Cucumovirus, Tobamovirus, Ilarvirus, Tobravirus, and Potexvirus), Picornavirus-like viruses (e.g., Picornaviridae, Caliciviridae, Comovirus, Nepovirus, and Potyvirus), minus-stranded viruses (e.g., Paramyxoviridae, Rhabdoviridae, Orthomyxoviridae, Bunyaviridae, and Arenaviridae), double-stranded viruses (e.g., Reoviridae and Bimaviridae), Flavivirus-like viruses (e.g., Flaviviridae and Pestivirus), Retrovirus-like viruses (e.g., Retroviridae), Coronaviridae, and other viral groups including, but not limited to, Nodaviridae.

A preferred RNA virus according to the invention is a virus of the family Flaviviridae, preferably a virus of the genus Filovirus, and especially a Marburg or Ebola virus. Preferably, a virus of the family Flaviviridae is a virus of the genus Flavivirus, such as yellow fever virus, dengue virus, West Nile virus, St. Louis encephalitis virus, Japanese encephalitis virus, Murray Valley encephalitis virus, Rocio virus, tick-borne encephalitis virus, and the like.

Also preferred is a virus of the family Picomaviridae, preferably a hepatitis A virus (HAV), hepatitis B virus (HBV), or a non-A or non-B hepatitis virus.

Another preferred RNA virus is a virus of the family Retroviridae (i.e., a retrovirus), particularly a virus of the genus or subfamily Oncovirinae, Spumavirinae, Spumavirus, Lentivirinae, and Lentivirus. An RNA virus of the subfamily Oncovirinae is desirably a human T-lymphotropic virus type 1 or 2 (i.e., HTLV-1 or HTLV-2) or bovine leukemia virus (BLV), an avian leukosis-sarcoma virus (e.g., Rous sarcoma virus (RSV), avian myeloblastosis virus (AMV), avian erythroblastosis virus (AEV), and Rous-associated virus (RAV; RAV-0 to RAV50), a mammalian C-type virus (e.g., Moloney murine leukemia virus (MuLV), Harvey murine sarcoma virus (HaMSV), Abelson murine leukemia virus (A-MuLV), AKR-MuLV, feline leukemia virus (FeLV), simian sarcoma virus, reticuloendotheliosis virus (REV), spleen necrosis virus (SNV)), a B-type virus (e.g., mouse mammary tumor virus (MMTV)), and a D-type virus (e.g., Mason-Pfizer monkey virus (MPMV) and “SAIDS” viruses). An RNA virus of the subfamily Lentivirus is desirably a human immunodeficiency virus type 1 or 2 (i.e., HIV-1 or HIV-2, wherein HIV-1 was formerly called lymphadenopathy associated virus 3 (HTLV-III) and acquired immune deficiency syndrome (AIDS)-related virus (ARV)), or another virus related to HIV-1 or HIV-2 that has been identified and associated with AIDS or AIDS-like disease. The acronym “HIV” or terms “AIDS virus” or “human immunodeficiency virus” are used herein to refer to these HIV viruses, and HIV-related and -associated viruses, generically. Moreover, an RNA virus of the subfamily Lentivirus preferably is a Visna/maedi virus (e.g., such as infect sheep), a feline immunodeficiency virus (FIV), bovine lentivirus, simian immunodeficiency virus (SIV), an equine infectious anemia virus (EIAV), and a caprine arthritis-encephalitis virus (CAEV).

A virus according to the invention also desirably is a DNA virus. Preferably, the DNA virus is an Epstein-Barr virus, an adenovirus, a herpes simplex virus, a papilloma virus, a vaccinia virus, and the like.

Many of these viruses are classified as “Biosafety Level 4” (i.e., World Health Organization (WHO) “Risk Group 4”) pathogens for which maximum containment facilities are required for all laboratory work. The ordinary skilled artisan, however, is familiar with and is capable of adhering to the safety precautions necessary for these viruses.

A “host cell” can be any cell, and, preferably, is a eukaryotic cell. Desirably, the host cell is a lymphocyte (such as a T lymphocyte) or a macrophage (such as a monocytic macrophage), or is a precursor to either of these cells, such as a hematopoietic stem cell. Preferably, the cells comprise a CD4+ glycoprotein on the cell surface, i.e., are CD4+. Desirably, however, a CD4+ T lymphocyte, which has been infected with the AIDS virus, has not yet become activated (i.e., preferably expression of nef has not yet occurred, and, even more preferably, CD4 gene expression has not been downregulated, as further discussed below). Moreover, a host cell preferably is a cell that lacks the CD4 marker, and yet is capable of being infected by a virus according to the present invention. Such a cell includes, but is not limited to, an astrocyte, a skin fibroblast, a bowel epithelial cell, an endothelial cell, an epithelial cell, a dendritic cell, Langerhan's cells, a monocyte, a hematopoietic stem cell, an embryonic stem cell, a cell that give rise to spermatozoa or an oocyte, a stromal cell, a mucosal cell and the like. Preferably, the host cell is of a eukaryotic, multicellular species (e.g., as opposed to a unicellular yeast cell), and, even more preferably, is a mammalian, e.g., human, cell.

A cell can be present as a single entity, or can be part of a larger collection of cells. Such a “larger collection of cells” can comprise, for instance, a cell culture (either mixed or pure), a tissue (e.g., endothelial, epithelial, mucosa or other tissue, including tissues containing the abovementioned CD 4 lacking cells), an organ (e.g., heart, lung, liver, muscle, gallbladder, urinary bladder, gonads, eye, and other organs), an organ system (e.g., circulatory system, respiratory system, gastrointestinal system, urinary system, nervous system, integumentary system or other organ system), or an organism (e.g., a bird, mammal, or the like). Preferably, the organs/tissues/cells being targeted are of the circulatory system (e.g., including, but not limited to heart, blood vessels, and blood, including white blood cells and red blood cells), respiratory system (e.g., nose, pharynx, larynx, trachea, bronchi, bronchioles, lungs, and the like), gastrointestinal system (e.g., including mouth, pharynx, esophagus, stomach, intestines, salivary glands, pancreas, liver, gallbladder, and others), urinary system (e.g., such as kidneys, ureters, urinary bladder, urethra, and the like), nervous system (e.g., including, but not limited to, brain and spinal cord, and special sense organs, such as the eye) and integumentary system (e.g., skin, epidermis, and cells of subcutaneous or dermal tissue). Even more preferably, the cells being targeted are selected from the group consisting of heart, blood vessel, lung, liver, gallbladder, urinary bladder, and eye cells. The target cells need not be normal cells and can be diseased cells. Such diseases cells can be, but are not limited to, tumor cells, infected cells, genetically abnormal cells, or cells in proximity or contact to abnormal tissue such as tumor vascular endothelial cells.

Vector

A “vector” is a nucleic acid molecule (typically DNA or RNA) that serves to transfer a passenger nucleic acid sequence (i.e., DNA or RNA) into a host cell. Three common types of vectors include plasmids, phages and viruses. Preferably, the vector is a virus, which includes the encapsidated forms of vector nucleic acids, and viral particles in which the vector nucleic acids have been packaged.

Desirably, the vector is not a wild-type strain of a virus, inasmuch as it comprises human-made mutations or modifications. Thus, the vector typically is derived from a wild-type viral strain by genetic manipulation (i.e., by deletion) to comprise a conditionally replicating virus, as further described herein. Optimally, the viral vector comprises a strain of virus that is of the same type as the wild-type virus causing the infection being treated, which, preferably, is one of the aforementioned wild-type viruses. Accordingly, preferably, the vector is derived from an RNA virus, even more preferably, the vector is derived from a retrovirus, and, optimally, the vector is derived from a human immunodeficiency virus. Such a vector derived from a human immunodeficiency virus is referred to generically herein as a “crHIV” vector.

A vector also, preferably, is a “chimeric vector,” e.g., a combination of a viral vector with other sequences, such as, for instance, a combination of HIV sequences with one or more other virus (which, desirably, is derived from a wild-type viral strain to comprise a conditionally replicating vector). In particular, HIV sequences desirably can be linked with sequences of a modified (i.e., non-wild-type) strain of adenovirus, adeno-associated virus, a virus from the Alphaviridae, a virus from the Flaviviridae, a virus from the Hepadnaviridae, a virus from the Papovaviridae, a virus from the Parvoviridae, a virus from the Herpesviridae, a virus from the Poxyiridae, a virus from the Paramyxoviridae, a virus from the Rhabdoviridae or a virus from the Retroviridae, including the Onco-retroviruses, the Spuma-retroviruses and the Lenti-retroviruses. Viruses or virus-like genomes derived or associated with these viral families are also included.

A preferred chimeric vector is when vector sequences (i.e. sequences either coding for proteins, fragments or non-coding sequences) derived from a non-Lentivirus are inserted into a Lentiviral vector. Preferably, non-HIV sequences are inserted into an HIV derived vector.

As encompassed herein, a vector can comprise either DNA or RNA. For instance, either a DNA or RNA vector can be used to derive the virus. Similarly, a cDNA copy can be made of a viral RNA genome. Alternatively, a cDNA (or viral genomic DNA) moiety can be transcribed in vitro to produce RNA. These techniques are well-known to those skilled in the art, and also are described in the following Examples.

A “conditionally replicating virus” is a replication-defective virus, which is defective only under certain conditions. In particular, the virus can complete its replicative cycle in a permissive host cell, and cannot complete its replicative cycle in a restrictive host cell. A “host cell” is a cell capable of being infected or actually infected with a wild-type strain of virus or a pseudotyped vector. Such wild-type virus infection can occur either before or after infection with a conditionally replicating virus according to the invention. Alternatively, a “host cell” is one that encodes wild-type viral or helper gene products necessary for viral replication. Thus, a conditionally replicating vector according to the invention is a virus (which preferably is the same type of virus as the infection being treated) that replicates only upon complementation with a wild-type strain of virus (or a helper) or when wild-type virus infects cells containing conditionally replicating vector genomes.

In a preferred embodiment, a vector comprises an RNA virus (e.g., a conditionally replicating HIV virus), which is introduced in the form of DNA. This preferred embodiment provides a replicating HIV-1 (crHIV) vector strategy that affords nonpathogenic crHIV-1 vector genomes with a selective advantage over pathogenic wild-type HIV genomes. Specifically, in cells containing both wild-type HIV and crHIV genomes, crHIV RNAs have a selective advantage for packaging into virions because they contain, for instance, ribozymes that cleave wild-type RNA, but not crHIV RNA. Such nonpathogenic crHIVs are able to spread to uninfected cells that are susceptible to HIV infection (e.g., CD4+ cells) in the presence of wildtype helper virus. In this manner, selective packaging and spread of crHIV interferes with wildtype HIV replication.

A further preferred embodiment is a non-pathogenic crHIV vector that is non pathogenic because it does not contain any combination of the viral accessory protein sequences (such as, but not limited to, Vif, Vpu, Vpr or Nef, or combinations or fragments thereof) that would make the vector pathogenic. Alternatively, the sequences may be present but transcriptionally silent or not translated. Optionally, the vector does not contain any combination of the regulatory protein sequences (such as, but not limited to, Tat or Rev, or fragments thereof) that would make the vector pathogenic. Alternatively, these sequences may be present but transcriptionally silent or not translated.

The vectors do, however, preferably contain any combination of the structural protein sequences (such as gag, or a fragment thereof), the enzymatic protein sequences (such as pol, or a fragment thereof), and/or the envelope protein sequences (such as env, or a fragment thereof) in a form capable of being either translationally active or silent. Therefore, the conditionally replicating vector may be able to replicate, but not replicate to levels that are pathogenic in a given host. Instead, the vector requires complementation with a helper component (such as a helper vector) containing the necessary sequences derived from the wild-type virus in order to replicate to sufficient levels in order to induce the required therapeutic, prophylactic or biological effect. Determining the precise combination of proteins or nucleotide sequences present in the vector and the helper to provide the optimal biological effect involves only straightforward application of simple screening processes involving addition and subtraction of various combination of nucleotide sequences into vector or helper constructs, which processes are routine to those skilled in the art.

Furthermore, the above nucleotide sequences can be modified or mutagenized in order to modify the biological effect, or for example, to decrease the chance for recombination with the helper construct. Numerous protocols for modification or mutation of vector and helper constructs to obtain more optimized constructs are well known in the art (e.g. Current Protocols in Molecular Biology, Harcourt Brace and Jovanovich, 2000; Molecular Cloning, Sambrook et al, Cold Spring Harbor Press, 1989; and Soong et al Nature Genetics 25: 436-439, 2000both incorporated here in their entirety).

The approach permitted with the above vectors is different from the use of live-attenuated (LA) vaccines that use replication-competent viruses that lack accessory proteins (see Daniel et al., Science, 258, 1938-1941 (1992); and Desrosiers, AIDS Res. & Human Retrovir., 10, 331-332 (1994)) because with LA vaccines, no effort is made to complement for deficiencies that, for example, prevent the efficient production of an effective immune response and yet maintain safety. For example, it is known that multiply deleted LA SIV vaccines cannot elicit an effective immune response, while singly deleted (nef negative) LA SIV vaccines are pathogenic in juvenile macaque apes (Baba et al, 1995).

An alternative approach to a LA HIV vaccine provided by the present invention as described above would be to use at least two vectors where at least one is a multiply deleted HIV vector and the second (helper) vector expresses all accessory proteins except Nef. Therefore, the multiply attenuated HIV vector could replicate in a conditional manner without causing disease while being complemented with Vif, Vpr and Vpu from the helper vector in a controlled manner. The first vector may be constructed to contain genetic antivirals so as to interfere with wild-type HIV replication and spread. Alternatively, such the first vector could be used to elicit an effective immune response against the wild-type virus. It will be plain to those skilled in the art that a simple screening process will permit evaluation of which actual combination(s) of sequences deleted from the first vector but present in the helper vector would permit the first vector to optimally provide a therapeutic or prophylactic response, and yet maintain safety by being non-pathogenic.

A further preferred embodiment of the conditionally replicating vector-helper system would use an HIV-1 vector to express gag, pol, env, tat and rev, while using a HIV-2 derived helper vector, for example, to express the Vif, Vpu, Vpr, and optionally Nef genes. The invention is not limited by this example because any combination of the above genes placed in any compatible vector combination, including reversed HIV-1 and HIV-2 or chimeric HIV formats, is equally possible. The expression of Tat from the HIV-1 backbone would transactivate both HUV-1 and HIV-2 LTRs for complementation with one another to produce vector particles that contained either the HIV-1 vector genomes or HIV-2 vector genomes. However, the two genomic RNAs would not effectively dimerize, therefore preventing the colocalization of both vector and helper genomes which would otherwise both be packaged into one virion particle. If co-packaged into the same virion particle, recombination between the vector and helper genomes to produce a replication competent vector (RCV) may occur during reverse transcription after the particle infects a subsequent target cell.

To further address the possible production of co-packaged constructs, the vectors may further contain one or more nucleic acid sequences that reduce the risk of any recombination. For example, the first vector may contain a ribozyme (or antisense/ribozyme) sequence specific for cleavage of the helper vector. Co-localization, in the same cellular, subcellular or extracellular location of such vectors would result in the cleavage of the helper vector to increase safety by destroying the dimerization or co-localization of the vectors, resulting in an inactive genome after recombination, or otherwise prevent the production of a RCV to emerge. This approach can be altered by including a ribozyme on the helper vector instead or further improved by the inclusion of ribozymes in both vectors to provide additional safety.

Alternatively, antisense molecules could be used to replace the above ribozymes, so that formation of a double stranded RNA hybrid would be rapidly degraded by cellular endonucleases. A further preferred embodiment is that the antisense sequences would be greater in length than the approximately 16 base positions of a ribozyme RNA used for targeting.

In a modified approach, the helper vector of the above example may be derived from a heterologous virus (such as any of the viruses and virus families described above, but preferably derived from an adenovirus, adeno-associated virus, a murine oncoretrovirus, or a non-HIV lentivirus) whereby the proteins could be expressed constitutively. Alternatively, the heterologous helper vector may be constructed to utilize an HIV-LTR for inducible or autoregulated expression of Tat, so as to transactivate HIV-1 vector expression. An advantage provided by a heterologous viral helper vector is the restrictive association between the genomes of the first and second vectors, thus further reducing the chance for recombination to generate a possible RCV.

In another embodiment, the vector contains an antisense sequence that is present on the helper genome or is inserted into the helper genome. A non-limiting example is seen with the pN1cptASgag vector, which is analogous to the pN1cptASenv vector except the antisense sequence is directed to the gag sequence present on the helper vector in addition, to or instead of, the env sequence present on wild-type HIV. The anti-gag antisense sequence is placed upstream of the splice acceptor site that is present downstream of the RRE sequence that is present in the vector. Therefore, the gag sequence would be packaged only in genomic and not subgenomic, or spliced, species of vector RNAs. Thus the helper genomes of intron containing helpers, such as those of the VIRPAC system, would be preferentially targeted to the spliceosome of the cell, while the genomic vector RNAs, that contain the anti-gag antisense sequence, would preferentially by-pass the splicing machinery.

The differential trafficking of the vector and helper genomes in a cell should result in minimal effects on vector titer, yet if the vector and helper genomes should serendipitously be co-localized, then the base-pairing hybridization of vector and helper genomes should result in the inactivation or destruction of such vector and helper genome containing particles and prevent vector-helper recombination to generate an RCV. In an alternate embodiment, the helper could be engineered to contain anti-U5 vector antisense sequences directed to the U5 sequences found in the conditionally replicating vector. Without limiting the nature of this aspect of the invention, the anti-U5 antisense sequences could be inserted distally to helper coding sequences, but before the transcriptional termination site. Hence, if vector and helper RNAs should serendipitously be co-localized and undergo co-packaging and possibly recombination, then the antisense sequences would destroy or inactivate such co-packaged viral particles and prevent recombination.

In yet another embodiment, the helper can contain target sequences for a first-nucleotide sequence present on the vector. A non-limiting example is by placing a sense env fragment into the helper of a two-plasmid pair that contained the pN1cptASenv vector. Therefore, if vector and helper RNAs should be co-localized, the env sense sequence in the helper would undergo base-pairing with the env antisense sequence in the vector to prevent recombination. The examples provided above are not meant to limit the invention to just one or two types of genetic antiviral sequence. It would be plain to anyone skilled in the art that numerous genetic antiviral sequences, as well as their cognate target sequences, could be inserted into vector and/or helper constructs. As a further non-limiting example, a pN1cptASgagASenv vector and a helper that contained the sense gag and env fragment sequences can be used in combination with each other to increase safety by reducing the likelihood of co-packaging and recombination to generate an RCV.

In another preferred embodiment for the expression of vector or helper components, the components are expressed transiently. One means of making the vector or helper express its genome or genomic components transiently is to construct the vector or helper vector using, for example, an integrase negative Pol gene. Such lentiviral integrase mutants are known in the art and have been reported not to be infectious (Hirsch et al 1989 Nature 341: 573-574). Therefore, the vector and/or helper genomes that are produced from integrase negative producer cells will not integrate but could be expressed in a transient manner so as to regulate the amount of vector or helper replication. Yet another means for transient expression from either the vector or the helper is to disrupt the AAT sites in the vector LTRs. These sites are responsible for active integration of the reverse-transcribed genomic vector DNA into the chromosome of the host cell.

A further means of accomplishing the above utilizes a fusion protein to package a functional integrase molecule into viral particles. In this embodiment, neither the conditionally replicating vector, such as a lentivector, or the helper vector would encode the integrase, but the functional integrase fusion protein would be available, by expression from another plasmid construct, such a helper vector, or from an integrated copy in the packaging cell used, during production or packaging of the helper construct, thus providing functional integrase activity upon infection. Alternatively, the integrase protein is provided in trans via expression from a helper construct that encodes it. In this embodiment of the invention, the conditionally replicating vector or the lentivector would not encode an integrase. The fusion protein may be, but is not limited to, a vpr-integrase fusion protein containing a protease cleavage site at the junction between the vpr and the integrase amino acid sequences.

The above description of a two vector-helper system should in no way limit the invention to two vector or helper constructs. Any combination of two, three, or more vectors and/or helpers to provide the components necessary for production of the vector may be used. Partitioning the necessary genomic elements into more vector or helper components will have the effect of increasing safety since it will be more difficult for multiple vector and helper genomes to generate an RCV via recombination. Safety can be further enhanced by constructing the vector(s) and helper(s) to contain little or no regions of homology between them, which is a further preferred embodiment. Partitioning the necessary genomic elements into multiple components may also further restrict replication of the conditionally replicating vector since it is less probable for more than two, in comparison to only two, genomes to be simultaneously present within a host cell. The optimal number of vector(s) and helper(s) required may be easily determined by simple screening of the different combinations and may vary depending upon the particular viral vector system that is used.

One simple, and non-limiting means, of limiting or removing regions of homology between vector(s) and helper(s) is by simply degenerating the nucleotide sequence, while maintaining the encoded amino acid sequence. Techniques to degenerate sequences are known in the art and routine. One preferred method is to humanize the sequences if the therapeutic use is in humans. Codon usage has been tabulated for primates and is described in Wada et al. (Nucleic Acids Research vol. 18 Supplement: 2367-2411, 1990), which is hereby incorporated as if fully set forth.

Degeneration may also be used to protect a helper construct from the effects of an agent designed to target a vector to be packaged with the helper. This may be simply accomplished by degenerating the cognate target sequence, if any, found on the helper construct. Additionally, degeneration of both the vector and helper constructs may be designed to reduce recombination with other viral sequences, including those of other wild-type sequences which may coincidentally be found with a vector or helper sequence in a cell. For example, the use of a vector and helper pair in a packaging system based upon HIV-1 and HIV-2 may be modified such that the vector and helper pair are degenerated for an endogenous retro-element as well. Thus degenerating the nucleotide sequence of either vector or helper would reduce colocalization of putative recombinants and thus reduce the risk of generating a replication competent virus between vector and helper genomes or between vector or helper genomes, and a competing genome such as an endogenous retro-element.

In particular, crHIV genomes are introduced into virally infected cells or uninfected cells. Infected cells supply the crHIV genome with proteins required for encapsidation and production of progeny virions. crHIV genomes are introduced into uninfected cells preferably either directly by transduction (e.g., this can be done, for instance, by liposome-mediated transduction of crHIV DNA, or by using a chimeric viral vector), or by infection of crHIV particles that result from transfection of wild-type HIV-infected cells. Uninfected cells containing an improved crHIV vector of the invention do not produce quantities of crHIV particles that are pathogenic to the host. Some embodiments of crHIV vectors cells not superinfected with wt-HIV will produce some crHIV particles, but at levels that are not pathogenic to the host. Cells containing any vector of the invention could remain susceptible to superinfection with wild-type virus, which would supply the proteins required for the further production of crHIV particles. In this sense, a conditionally replicating vector according to the invention, in the presence of a complementary wild-type superinfection, also functions as a type of “viral delivery vector” whereby, for example, multiple rounds of crHIV infection (i.e., in the presence of concurrent infection with wild-type HIV) can ensue. Such a vector provides a source of virus for more than one round of viral replication and thus infection of other cells, or multiple rounds of replication to levels that would elicit a biological response such as an immune response. This improvement is in contrast to other vectors, such as those used with standard packaging cell lines, and which provide for only a single round of replication, or multiple rounds of replication that are either insufficient to elicit an appropriate biological response or are pathogenic to the host.

If desired (e.g., to facilitate use of the vector in vitro), wild-type viral gene products can be co-supplied to a cell infected with the conditionally replicating vector. Wild-type viral gene products can be supplied not only by co-infection with a wild-type viral strain (or a cDNA or provirus of a RNA virus), but also by supplying them to a cell in the form of their genes subcloned in an expression vector, e.g., a helper expression vector (“helper” or “helper vector”), that is capable of imparting on a host cell transcription or translation of the sequences (regulatory or structural), or, alternatively, the gene products can be supplied exogenously, i.e., by adding the protein products to the cell.

For example, a crHIV vector may be constructed that contains all the proteins from wildtype HIV except for, for example, the Tat coding sequence. Instead of using a helper expression construct that expresses Tat, the Tat protein itself could be used as a helper. The advantage of using the protein instead of a helper construct is that there is no theoretical possibility of generating a wild-type virus since no nucleic acid sequences are present for recombination. Thus, the crHIV can be propagated by using the Tat protein and not by a helper nucleic acid construct per se. Tat 1 or Tat 2 (corresponding to the first, or the first and second exons of Tat, respectively) may be used as the helper. Methods to produce and purify such proteins are well known in the art.

In one preferred embodiment, a chimeric Tat protein is used. Tat has be made into a fusion protein (e.g. Tat-VP16) and shown to retain its transactivating activity of the HIV-LTR promoter. Other chimeric Tat proteins could be created to enhance the desired biological effect. For example, if the desired biological effect is to enhance the immune response, then a Tat-GM-CSF fusion protein could be constructed. This approach is not limited to one type of chimeric protein and it may be desirable for at least a second chimeric (or non-chimeric) Tat protein to be added (e.g. Tat-IFN-alpha, Tat-TNF-alpha, Tat-IL-4, Tat-G-CSF, TNF-alpha, GM-CSF, IL-4, G-CSF, IFN-alpha to name but a few possibilities contemplated). It would be easily apparent to anyone in the art to construct and test other equivalent chimeric proteins and screen them for the ability to enhance an immune response while at the same time providing the helper function for the vector. Another preferred embodiment is a Tat-Rev fusion protein construct, or a native HIV Tat-Rev fusion protein, Trev, for example. Therefore, the above description of chimeric Tat proteins is not limited to the inclusion of only cytokine or cellular proteins, but viral proteins (producing homologous or heterologous viral fusion proteins) may also be used in the invention depending upon the desired biological effect.

With respect to the “helper vector,” its expression can be cell specific or not cell-specific and it can be introduced into a host cell in concert with a conditionally replicating viral vector as defined herein and, thereby, enable continuous replication of the conditionally replicating viral vector.

The “helper vectors” of the invention can supply the necessary gene products for replication by either a single vector or multiple vectors. Such vectors are preferably used in a transient transfection system. Alternatively, the gene products may also be integrated into the genome of the host cell or cell line. According to the invention, a single vector or plasmid (which may be mono-, bi-, or multi-cistronic in a host cell) is preferable due to the increased vector titers resulting from the enhanced likelihood of co-transfecting the helper vector and conditionally replicating vector into the same cell. The reduced likelihood of simultaneously transfecting more than two different vectors into the same cell makes the use of a single helper vector more desirable. Decreasing the number of vectors or plasmids involved in a transfection system is also cost-efficient as fewer plasmids must be produced as compared to a conventional three plasmid transfection system to package retroviral constructs.

As used herein, “complementation” refers to the nongenetic interaction of viral gene products from different sources in cells. Specifically, with a mixed infection, complementation comprises an enhancement in the viral yield of one or both parental genomes, while the genotypes of the parental genomes remain unchanged. Complementation can be nonallelic (i.e., intergenic, wherein mutants defective in different functions assist each other in viral replication by supplying the function that is defective in the other virus) or allellic (i.e., intragenic, wherein the two parents have defects in different domains of a multimeric protein).

Desirably, the types of cells that can be transfected (transduced) with crHIV DNA (i.e., by liposomes or by using a heterologous vector to make a chimeric vector, as described above) can be either HIV-infected or uninfected cells. HIV infected cells can be activated or unactivated. If they are activated, they will immediately transcribe wild-type HIV RNA and crHIV RNA, resulting in selective packaging of crHIV RNA into progeny virions. If HIV-infected cells are not activated, the crHIV DNA will reside in them until they become activated (e.g., through stimulation by mitogens, antigens, and the like), resulting again in selective packaging of crHIV RNA into progeny virions. Both activated and unactivated uninfected cells that are transfected with crHIV DNA will not produce virions until they become superinfected with wild-type HIV and activated by stimulation, resulting again in selective packaging of crHIV RNA into progeny virions.

One embodiment of a host cell transduced with a vector of the invention is where the cell contains the maximum number of vector copies that is not significantly toxic to either the host cell or host organism containing the cell. Another embodiment of a host cell transduced with a vector is where the cell contains at least one copy of the vector, but preferably contains the minimum number of vector copies required for generating the biological effect. The copy number in a cell can be determined by, for example, TaqMan PCR, and the acceptable copy number or range of copy numbers, appropriate for both the desired biological effect and the lack of toxicity can be easily determined by those skilled in the art. The acceptable copy number will vary depending upon factors including the cell type that is transduced, the nature of the vector (e.g. the viral origin) and the desired biological effect, but remains easily determined by routine screening by those familiar with the art.

Superinfection of cells containing crHIV genomes (e.g., as a result of transfection or infection) occurs because crHIV genomes do not encode viral proteins that block superinfection (such as env and nef). The resulting crHIV virions can infect uninfected cells because the viral particles contain the reverse transcriptase molecule, which all HIV particles carry so that they can create a DNA provirus from their genomic RNA. This process is called reverse transcription. Once crHIV virions infect uninfected cells, they can undergo reverse transcription and produce a provirus from their genomic RNA. Thus, these cells are the equivalent to those uninfected cells that are directly transduced with crHIV DNA. They cannot produce crHIV particles until these cells become superinfected with wild-type HIV and become activated, then once again, selective packaging of crHIV RNA into progeny virions occurs. It is possible that crHIV particles could also infect some cells that are already infected with HIV (see, e.g., Yunoki et al., Arch. Virol., 116, 143-158 (1991); Winslow et al., Virol., 196, 849-854 (1993); Chen et al., Nuc. Acids Res., 20, 4581-4589 (1992); and Kim et al., AIDS Res. & Hum. Retrovir., 9, 875882 (1993)). However, for this to occur, these HIV-infected cells must not express proteins that down-regulate CD4 expression, because this will prevent the crHIV virions from infecting these cells. Activated, HIV-infected cells generally down-regulate CD4 expression. Accordingly, HIV-infected cells that are not activated are potentially susceptible to crHIV superinfection and, thus, could be another source for crHIV particle production With a preferred crHIV vector according to the invention, the vector comprises sequences required for RNA transcription, tRNA primer binding, dimerization and packaging, and either lacks sequences encoding proteins that block superinfection with wild-type HIV (e.g., nef or env proteins) or comprises such sequences but they are either not transcribed or not translated into functional protein, such that their expression is deemed “silent.” Even more preferably, the vector lacks the region or sequences coding the region of wild-type HIV from within the gag coding sequence to and including the nef gene. Optimally, however, the vector does comprise the rev responsive element (RRE), which is cloned into the vector in the region of the deletion or some other convenient region. Such a preferred HIV vector is said to “lack the region or sequences coding the region” inasmuch as this vector can be administered in its RNA manifestation, or, alternatively, as DNA, as previously described.

According to the invention, in order to achieve successful complementation in a system that utilizes a helper vector in conjunction with the conditionally replicating vector, any viral component necessary for viral packaging but not expressed from the conditionally replicating vector must be provided by the helper vector. Thus, as long as the two or more different vectors together possess a full complement of necessary viral components, the composition of the individual vectors is subject to many permutations. Each of these permutations is encompassed within the present invention. As an example, the gag and pol genes necessary for successful packaging and replication may both be incorporated either on the helper vector or the conditionally replicating vector, or the two genes may be individually present on one of each vector. Furthermore, a single helper vector construct may include both the gag/pol and env sequences, including heterologous env sequences, under the control of a single set of transcriptional regulatory elements, including the promoter, or under the control of separate promoter elements. For example, an LTR may be used to express gene products in the vectors of the invention. The use of different promoters, including inducible promoters, permits the possibility of differential regulation of the gag/pol and env sequences, for example. Thus, the env sequence, especially a heterologous env sequence, such as VSV-G envelope sequence, may be expressed at greater levels.

Vector construction is well-known to those skilled in the art. These techniques can be used to construct both the conditionally replicating vector and the helper vector. Thus, as used herein, the term “vector” can refer to both vector types. Additionally, the term vector encompasses any Lentiviral vector without necessarily being a conditionally replicating vector. Such vectors may also be referred to as generic Lentiviral vectors. In preferred embodiments of the invention, however, the vector is a conditionally replicating Lentiviral vector. For instance, and as described in Example 1, the DNA manifestation of a RNA virus, such as HIV, is cleaved using restriction enzymes to excise HIV encoding sequences from within the gag coding region to within the U3 region, following the nef gene. A cloning cassette comprised of a polylinker containing multiple restriction sites is inserted into the region of the deletion prior to ligation to provide convenient restriction sites for cloning into the vector. A DNA fragment containing RRE is subcloned into one of these sites. The resultant vector produces a truncated gag transcript, and does not produce wild-type Gag protein, or any other wild-type HIV proteins. Moreover, it is not necessary that the vector express even the truncated gag protein inasmuch as the gag translation initiation sequence can be mutated to prevent its translation.

Using the same approach, the crHIV sequences can be linked to other sequences, such as those of a virus or other vector, to derive a chimeric vector as described above. For instance, the crHIV sequences can be ligated to those of Sindbis virus, AAV, adenovirus, or amphotropic retrovirus to name but a few. Additional viral sequences which may be used include those of Herpes, Pox and the other viruses described herein, including any virus that can be used to provide for delivery of the crHIV sequences. Such a chimeric vector can be introduced into the cell either using the conjoined virus's mechanism for cell entry (e.g., receptor-mediated endocytosis for adenovirus) or other means, e.g., liposomes.

Preferably, according to the invention, a vector (i.e., a conditionally replicating virus that preferably is a crHIV vector) comprises at least one nucleic acid sequence, the possession (i.e., presence, transcription or translation) of which confers a selective advantage. There are two types of such nucleic acid sequences contemplated for inclusion in the vector: (1) a nucleic acid sequence, the possession of which optimally confers a selective advantage for viral replication and spread to a vector comprising such a sequence over a wild-type strain of virus (i.e., preferably, a wild-type strain from which the vector was derived, and which does not comprise the sequence), and (2) a nucleic acid sequence, the possession of which optimally confers a selective advantage to cells infected with a vector comprising the sequence as compared with cells infected, or uninfected, with a wild-type strain of virus (i.e., preferably, a wild-type strain from which the vector was derived (and also, for example, a helper-expression vector that promotes vector replication and/or function in an uninfected host cell), and which does not comprise the sequence) by, for example, promoting cell survival, promoting vector particle production and/or propagation, promoting the production of crHIV vector virions from crHIV vector-producing cells, inducing apoptosis, facilitating protein production or promoting immunological function or targeting, so as to achieve a desired prophylactic, therapeutic or biological outcome. Each of these sequences, or a plurality of each of these sequences, i.e., a sequence that alone or in combination with another factor(s), promotes the propagation of the vector and/or promotes a particular host cell function so as to enable a favorable prophylactic, therapeutic and/or biological outcome, can be included in the vector, either in the absence or the presence of the other sequence, i.e., the vector can comprise “at least one nucleic acid sequence” and “at least one additional nucleic acid sequence.”

A third type of nucleic acid sequence is one that confers a phenotype upon the host cell that diminishes, minimizes, or prevents viral infection. Such nucleic acid sequences include those that encode a gene product which, when expressed, protect the host cell from viral infection. For example, individuals homozygous for the Δ32 allele of the CCR5 protein were protected against HIV-1 infection due to the lack of a major co-receptor for HIV-1 entry into cells expressing the truncated CCR5 protein on their surfaces. Studies with heterozygous individuals have indicated that the Δ32 allele has a transdominant effect on wildtype (wt) CCR5 cell surface expression. This may be due to the ability of the Δ32 allele to dimerize with the wt protein and prevent its correct expression on the cell surface.

The Δ32 allele contains a deletion of 32 nucleotides which causes a frameshift mutation to result in a truncated CCR5 protein. Since this truncation gives rise to 31 amino acids in the C-terminus of the Δ32 allele which are not present in the wt protein and may generate an undesirable immune response if presented on the cell surface, the present invention includes the expression of a truncated CCR5Δ32 mutant protein (CCR5Δ32T) that lacks the potentially antigenic 31 amino acids. This protein would retain its ability to confer a transdominant effect to provide cells with protection against HIV infection. The sequence encoding CCR5Δ32T is readily generated by introducing a stop codon immediately after the last amino acid common to both wt CCR5 and CCR5Δ32 (after the tyrosine at position 184).

In a further embodiment, another third type of nucleic acid sequence is one that further enhances the conferred phenotype of diminishing, minimizing or preventing viral infection. Such a nucleic acid sequence can encode for a genetic antiviral that is, for example, targeted to a cellular gene. For example, the vector could express the above CCR5Δ32T mutant protein and in addition express antisense or ribozyme molecules, from a U1 promoter and contained within U1 snRNA (described in U.S. Pat. No. 5,814,500), for example, that is targeted to the wild-type CCR5 molecule. The antisense region that targets wild-type CCR5 would be degenerated in vector borne CCR5Δ32T sequence so as to make the transdominant mutant RNA resistant to the effects of the anti-CCR5 antisense or ribozymes molecules, but yet be translated into the correct amino acid sequence to produce functional CCR5Δ32T protein.

The above method of simultaneously expressing a ribozyme or antisense targeted to a gene product and degenerating the gene in the targeted sites to deter ribozyme or antisense binding or cleavage is not limited to CCR5 or even a molecule that can minimize virus replication. Another non-limiting example for therapeutic application is the targeting of an oncogenic protein, such as the Bcr-Abl fusion protein which is the etiologic agent for Chronic Myelogenous Leukemia. A vector construct containing the Bcr or Abl gene, or both, could express these genes simultaneously with the expression of an antisense or ribozyme that is targeted to any site along the Bcr-Abl transcript. Therefore, the abnormal Bcr-Abl fusion transcript would be destroyed while the native Bcr or Abl, or both, would be expressed to relieve the abnormal defect. Thus, the Bcr or Able genes would have a selective advantage for amplification over the wild-type or abnormal Bcr-Abl fusion gene. A preferred vector would express the Bcr and/or Abl construct from their native promoters, while the anti-Bcr-Abl ribozymes or antisense would be expressed from the U1 promoter and contained within U1 snRNA sequences.

The above Bcr-Abl example does not limit the invention to targeting oncogenes, and it would be obvious to one skilled in the art that this approach could be used for therapeutic or prophylactic substitution of any expressed abnormal gene, undesired gene, or even a desired gene(s) of interest. The target gene can be homologous or heterologous to the modified gene that is resistant to the effects of the genetic antiviral molecule. Other disease states that can be targeted by this method would be obvious to those skilled in the medical and clinical arts. For example, molecular targets for treating blood diseases by practice of the invention disclosed herein are described in “The molecular basis of blood diseases” (by Stamatoyannopoulos, Majerus, Perlmutter & Varmus, 3rd Ed., Philadelphia: W B Saunders Co., copyrighted 1987, 1994 and 2001, all of which are hereby incorporated by reference).

Nor should the invention be limited to clinical applications. The approach may be used to determine the function of a gene sequence of interest. For example, a gene sequence of interest modified in a domain that is of interest may be simultaneously expressed with an antisense or ribozyme that inhibits or destroys expression of any unmodified gene sequence by targeting the corresponding unmodified region. Thus, the modified gene sequence of interest would have a selective advantage for amplified expression over the unmodified gene sequence, permitting determination of the function of the modified gene sequence or its encoded gene product by assay procedures known in the art.

A “nucleic acid” is as previously described. A “nucleic acid sequence” in particular comprises any gene or coding sequence (i.e., DNA or RNA) of potentially any size (i.e., limited, of course, by any packaging constraints imposed by the vector), the possession of which confers a selective advantage, as further defined herein. A “gene” is any nucleic acid sequence coding for a protein or a nascent mRNA molecule (regardless of whether the sequence is transcribed and/or translated). Whereas a gene comprises coding sequences as well as noncoding sequences (e.g., regulatory sequences), a “coding sequence” does not include any noncoding DNA.

1. Nucleic acid sequence, the possession of which confers a selective advantage in a host cell to a vector comprising such a sequence over a wild-type strain of virus or a competing genomic sequence that would interfere or affect vector amplification.

A nucleic acid sequence, which confers a selective advantage to a vector in a host cell over a wild-type strain of virus, preferably is any sequence that allows viral particles propagated from the vector to be selectively produced or packaged as compared with viral particles propagated from the wild-type virus. Such sequences include, but are not limited to, a sequence that results in an increase in the number of vector genomes produced intracellularly as compared with wild-type genomes, and an antiviral nucleic acid sequence. Such sequences also include, but are not limited to, a sequence that results in an increase in the number, property or condition of vector genomes produced intracellularly as compared with wild-type genomes that are not derived from the cognate wild-type virus.

The first category of nucleic acid sequences that confer a selective advantage in a host cell to a vector containing the sequence as compared with a wild-type strain of virus are sequences such as a promoter. A “promoter” is a sequence that directs the binding of RNA polymerase and thereby promotes RNA synthesis, and that can comprise one or more enhancers. “Enhancers” are cis-acting elements that stimulate or inhibit transcription of adjacent genes. An enhancer that inhibits transcription also is termed a “silencer.” A silencer may also be an “insulator” element as is found in DNAse hypersensitive sites of the chicken erythroid insulator element. Enhancers differ from DNA-binding sites for sequence-specific DNA binding proteins found only in the promoter (which also are termed “promoter elements”) in that enhancers can function in either orientation, and over distances of up to several kilobase pairs (kb), even from a position downstream of a transcribed region.

Accordingly, and preferably, the promoter (e.g., the long-terminal repeat (LTR)) of a conditionally replicating HIV vector is modified such that the vector is more responsive to certain cytokines than is the wild-type HIV strain. For instance, a modified HIV promoter is available that demonstrates increased transcriptional activity in the presence of interleukin-2. Incorporation of this promoter into a vector and introduction of the vector into wild-type, HIV-infected cells preferably results in increased production and packaging of progeny virions from the vector genome as compared with the wild-type HIV genome. Other cytokines and/or chemokines (e.g., including, but not limited to, interferon-alpha, tumor necrosis factor-alpha., RANTES, and the like) similarly can be employed to promote selective packaging of virions encoded by the vector. The placement of elements that make the HIV-LTR responsive to cytokines by no means restricts the scope of the invention to such embodiments. Any nucleotide sequence may be inserted into the HIV-LTR to modify the responsiveness of this promoter. A list of factors that bind to DNA sites that could be inserted in the HIV-LTR may be found at http://transfac.gbf.de/TRANSFAC/lists/browse.html, accessed on Sep. 8, 2000, which is hereby incorporated by reference as if fully set forth. The listing of DNA binding factors for Homo sapiens (http://transfac.gbfde/TRANSFAC/lists/factor/species/humanHomosapiens.html) as accessed on Sep. 8, 2000 is similarly hereby incorporated by reference.

Similarly, silencers or insulators can also be inserted into the promoter or enhancer regions of a native or modified retroviral LTR, such as the HIV-LTR, so as to tightly regulate expression from this promoter. In certain cases, inserting elements into the HIV-LTR may place the vector at a selective disadvantage with respect to infection with a wild-type virus. However, the application of this type of vector may be not to compete with wild-type virus for packaging into progeny virions, but rather to express a nucleic acid sequence of interest (or “payload gene”) that, for example, inhibits HIV replication without competition. In this case the first nucleic acid sequence does not give the vector a selective advantage, but must in the very least, for example, inhibit recombination between the vector and the helper during the process of vector production.

The second category of a nucleic acid sequence that confers a selective advantage to a vector containing the sequence as compared with a wild-type strain of virus includes, as a preferred nucleic acid sequence, an antiviral nucleic acid sequence. “Antiviral agents” are categorized by their mode of action, e.g., inhibitors of reverse transcriptase, competitors for viral entry into cells, vaccines, protease inhibitors, and genetic antivirals. “Genetic antiviral agents” are DNA or RNA molecules that are transferred into cells and affect their intracellular targets either directly (i.e., as introduced intracellularly) or after their conversion to either RNA or protein (reviewed by Dropulic et al. (1994), supra). A genetic antiviral sequence also is a preferred nucleic acid sequence. Genetic antiviral agents include, but are not limited to, antisense molecules, RNA decoys, transdominant mutants, toxins, modifiers and modulators of RNA and protein splicing, EGS (solely or in direct association with M1, U1, PRE or CTE elements), immunogens, RNAi (see Zamore et al. Cell 101:25-33, 2000), nucleotide sequences or molecules that modify or modulate splicing, constitutive transport elements (CTE), nuclear import and export factors, DNA integration factors, RNA stabilizing or destabilizing elements, post-transcriptional regulatory elements (PRE), proteins that interfere with virus replication, interferons, toxins, immunogens (defined by any gene that is immunologically related), antibodies (whole antibodies, single-chain antibody or ligand display molecules), ribozymes, or any element that affects any aspect of vector or wild-type virus replication and ribozymes. Desirably, a genetic antiviral is an antisense molecule, a transdominant mutant, an immunogen, and a ribozyme. Accordingly, a preferred nucleic acid sequence that confers a selective advantage to a vector over a wild-type strain of virus is that of a genetic antiviral agent selected from the group consisting of an antisense molecule, an immunogen, and a ribozyme.

A genetic antiviral agent as used in the present invention are limited to only targeting a viral sequence, especially when they are not placed in conditionally replicating viral vectors. As a non-limiting example, a genetic antiviral sequence may be placed in a lentiviral vector such that the agent is directed to viral or non-viral targets, for example, cellular, bacterial or parasitic targets. In this case the lentiviral vector contains a gene of interest operably linked to an unmodified or modified HIV-LTR (to expressed unspliced or spliced RNA) and in addition contains a genetic antiviral agent or sequence that is linked to a promoter sequence that is not operably linked to the HIV-LTR. A preferred sequence is an antisense or ribozymes that is chimeric with a snRNA, preferably the U1, U2, U3, U4, U5 or U6 snRNA.

Also, the genetic antiviral agent is not limited to a single or single type of sequence present in either a vector or helper sequence. Two or more agents may be simultaneously present in a vector or helper construct, either distally located or preferably linked in tandem. Preferably two or more different genetic antiviral agents are linked in tandem. An additional genetic antiviral agent embodiment for use in the invention is any that links two different types of genetic antiviral agents.

An “antisense molecule” is a molecule that “mirrors,” based on basepairing rules and the availability of basepair “wobble,” a short segment of a gene whose expression is to be blocked. An antisense molecule directed against HIV hybridizes to wild-type HIV RNA, allowing its preferential degradation by cellular nucleases. Antisense molecules preferably are DNA oligonucleotides, desirably of about 20 to about 200 base pairs in length, preferably about 20 to about 50 base pairs in length, and, optimally, less than 25 base pairs in length. An antisense molecule can be expressed from crHIV RNA that preferentially binds to genomic wild-type RNA, thereby providing the crHIV RNA with a selective advantage for packaging into progeny virions.

Antisense molecules expressed in vectors as RNAs preferably are at least 20 bases up to any size, but preferably up to 2000 bases in length, more preferably about 50 to 500 bases in length. Such antisense molecules preferably bind to genomic wild-type RNA and not vector RNA, providing the vector with a selective advantage over the wild-type virus. A preferred region for generating an antisense molecule in an HIV derived vector, for example, is from the envelope region (env), the Tat or Rev protein regions, the accessory gene regions (Vif, Nef, Vpr, Vpu), regions of Gag that are 3′ to the Nsi I restriction enzyme site on pNL4-3, and the regions of Pol that do not contain the a 545 base region in Pol as set out below.

An “immunogen” refers to any immunologically related gene and its encoded gene product. Although historically the term “immunogen” was used in reference to an adjuvant used for vaccination, the terms “immuno-” and “-gen” is used herein to refer to an immunologically related gene and its encoded gene product. For example an immunogen can encode a single-chain antibody (scAb) directed to a viral structural protein, or it can encode an antibody that is secreted by the cell extracellularly. An immunogen is transferred as nucleic acid and expressed intracellularly. Similarly, an immunogen also can encode any antigen, surface protein (including those that are class-restricted) or display-like antibody, which facilitates vector and/or host cell selection. In a preferred vector, the nucleic acid sequence comprises a scAb encoding sequence that binds to wild-type HIV Rev protein. This preferably prevents maturation of Rev protein by resulting in its withholding in the endoplasmic reticulum. Specifically, Rev proteins, alone or in combination with other cellular factors, export unspliced and singly spliced HIV RNA from the nucleus to the cytoplasm by binding to the RRE (the highly structured cis-acting RNA target sequence for Rev, termed Rev responsive element) and then oligomerizing to surround the HIV RNA. HIV RNAs that are complexed with Rev are exported into the cytoplasm and bypass the cell's splicing machinery. Thus, if wild-type Rev does not bind to the wild-type RRE, then wildtype HIV RNAs are not exported into the cytoplasm, and are not encapsidated into progeny virions.

Optimally, the vector containing the scAb nucleic acid sequence further comprises a modified RRE sequence, and encodes a mutated Rev protein that recognizes the modified, but not the wild-type, RRE. Accordingly, in cells containing wild-type HIV and a vector comprising the scAb nucleic acid sequence, the vector preferentially is packaged into virions. A similar strategy preferably is employed wherein proteins of the wild-type HIV matrix or nucleocapsid (i.e., or any protein involved in protein/RNA interactions that affect encapsidation of viral RNA) are the targets of the scAb.

A “ribozyme” is an antisense molecule with catalytic activity, i.e., instead of binding RINA and inhibiting translation, ribozymes bind RNA and effect site-specific cleavage of the bound RNA molecule. Generally, there are four ribozyme groups: the Tetrahymena group I intervening sequence, EGS (external guide sequence), and the hammerhead and hairpin ribozymes. However additional catalytic motifs also exist in other RNA molecules, e.g., hepatitis delta virus and ribosomal RNAs in fungal mitochondria.

A preferred ribozyme is a ribozyme in which the catalytic domain cleaves a 3′-nucleotide NUH sequence, wherein N can be any nucleotide (i.e., G, A, U or C), and H can be either an A, C or U. However, inasmuch as the sequence that is cleaved most efficiently by such ribozymes is a GUC site, preferably the NUH sequence comprises a GUC site.

Desirably, such a ribozyme cleaves in a region of a wild-type strain of virus or its transcripts, but does not cleave in a region of a vector or its transcripts. The ribozyme cleaves the virus or its transcripts in the sense that such a virus or vector can be either RNA or DNA, as previously described. By cleavage “in a region” is meant cleavage in a targeted region, i.e., preferably a region of the virus that is necessary for viral propagation. Desirably, the vector has been modified so that this particular region being targeted (i.e., if present in the vector at all) is not cleaved by the ribozyme. Optionally, the ribozyme can cleave the vector, so long as cleavage does not occur in a region required for propagation of viral, e.g., crHIV particles.

Optimally, the ribozyme is encoded by a sequence selected from the group consisting of SEQ ID NO:3 (i.e., CACACAACACTGATGAGGCCGAAAGGCCGAAACGGGCACA) and SEQ ID NO:4 (i.e., ATCTCTAGTCTGATGAGGCCGAAAGGCCGAAACCAGAGTC). Whereas SEQ ID NO:3 comprises a ribozyme that is targeted to the +115 site (i.e., in terms of the number of bases downstream from the start of transcription) of the wild-type HIV U5 region, SEQ ID NO:4 comprises a ribozyme that is targeted to the +133 site of the wild-type HIV U5 region.

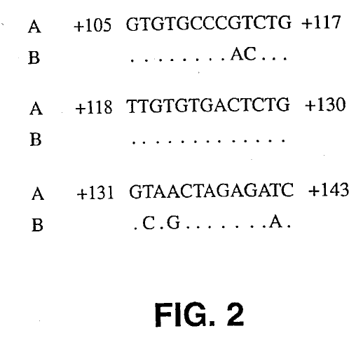

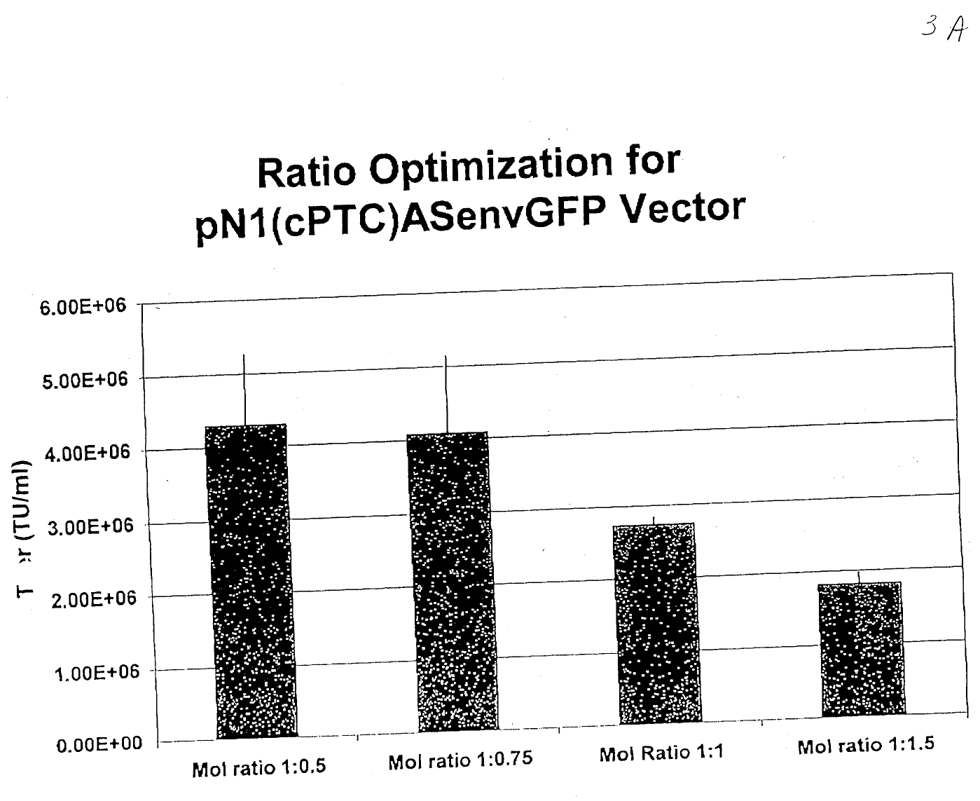

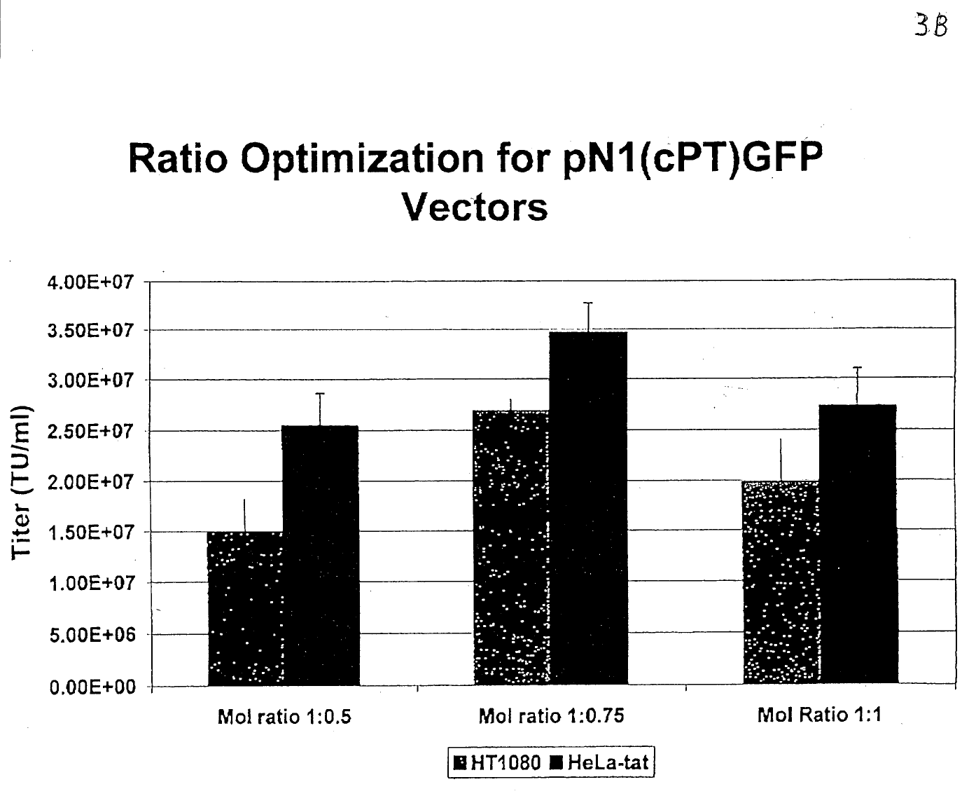

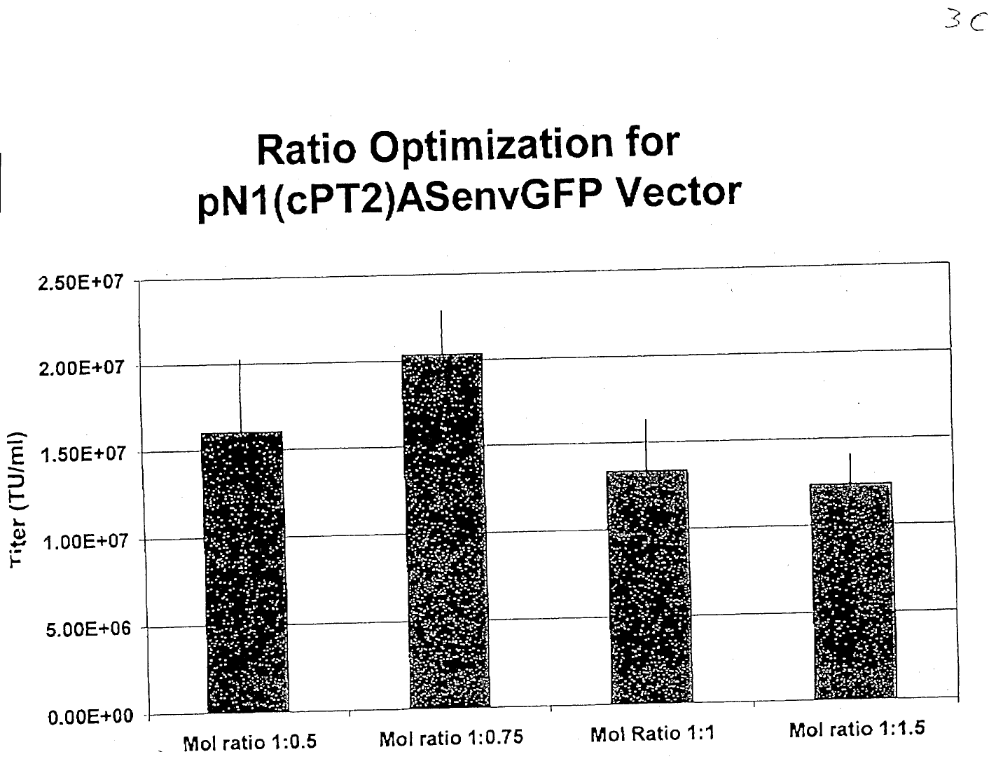

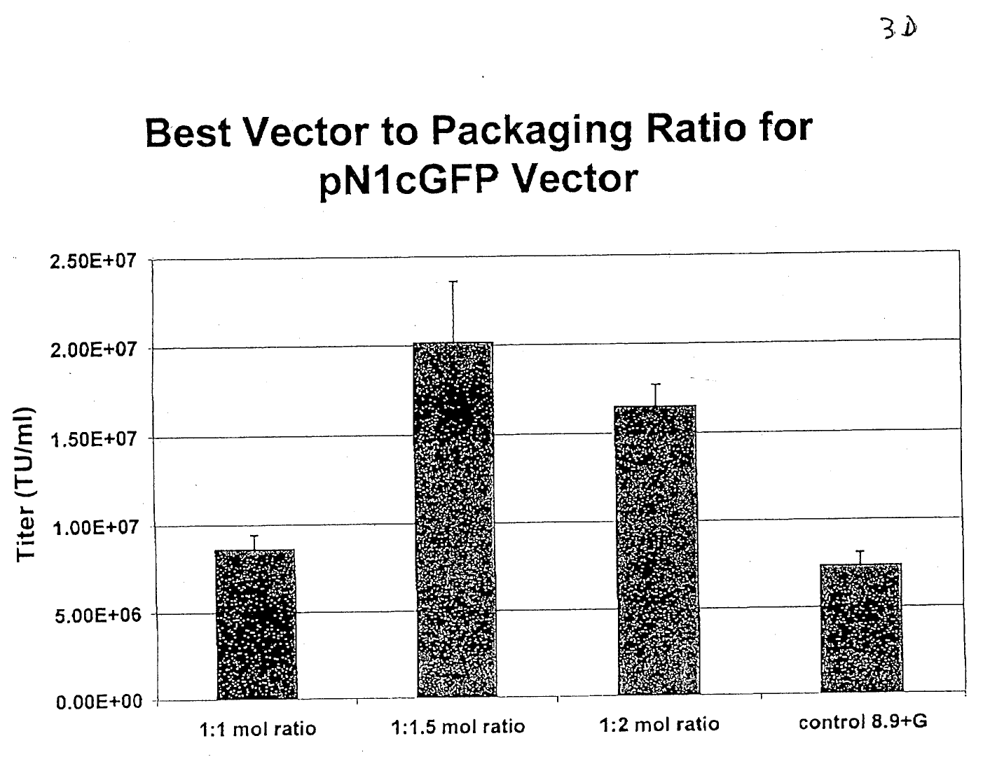

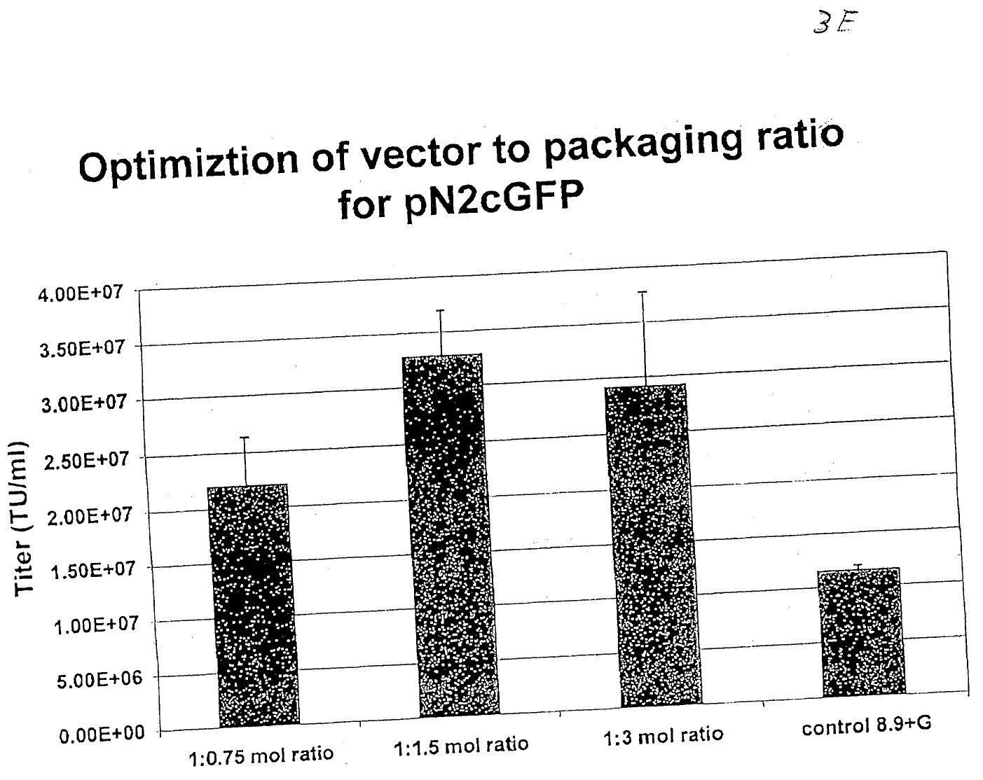

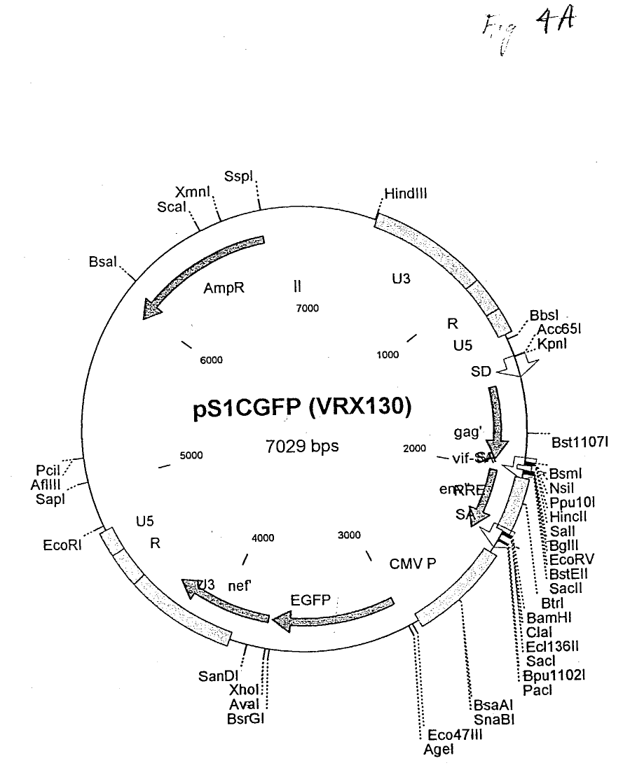

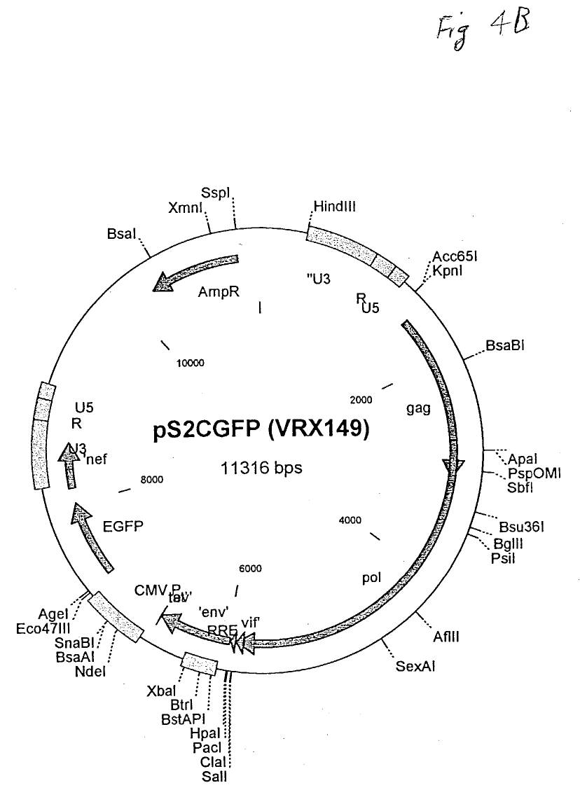

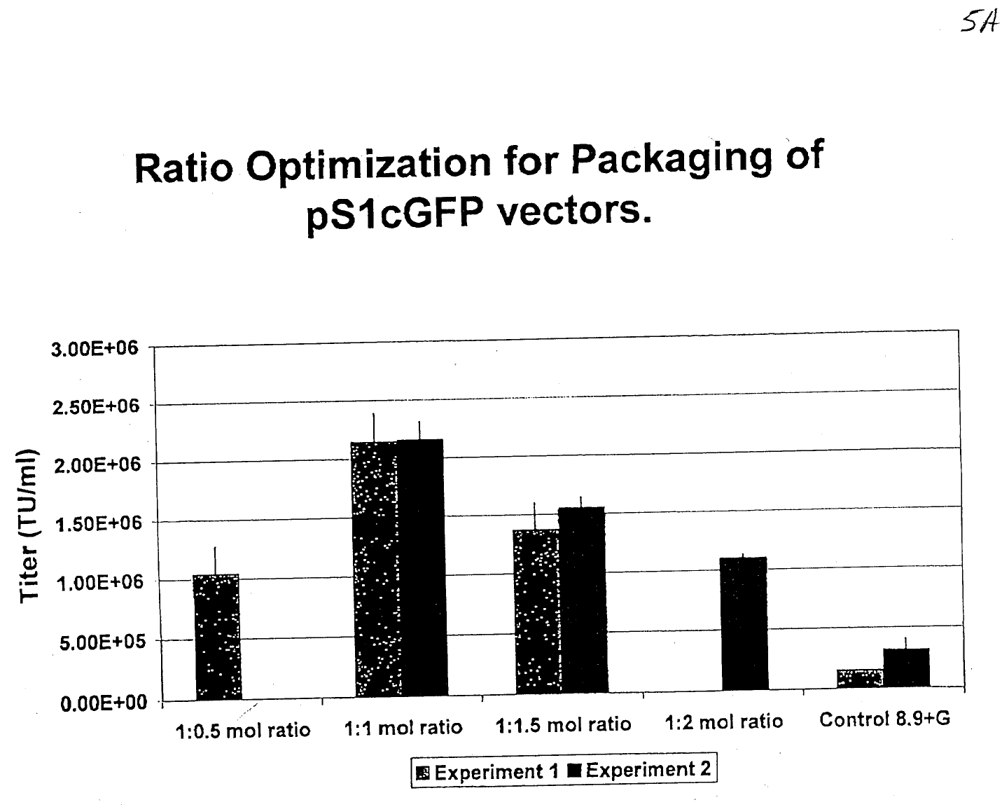

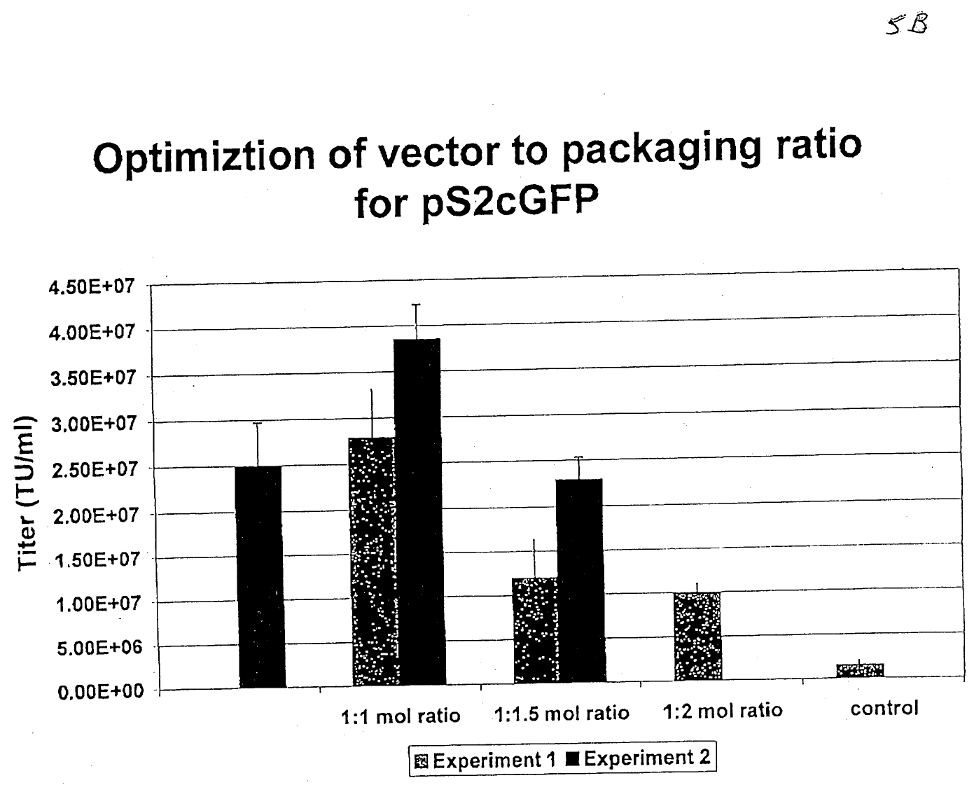

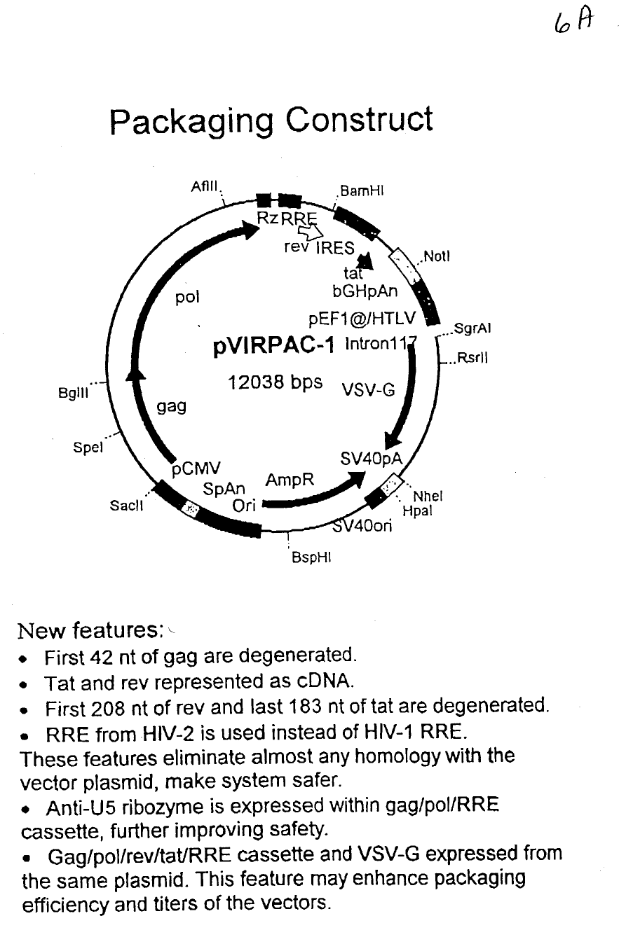

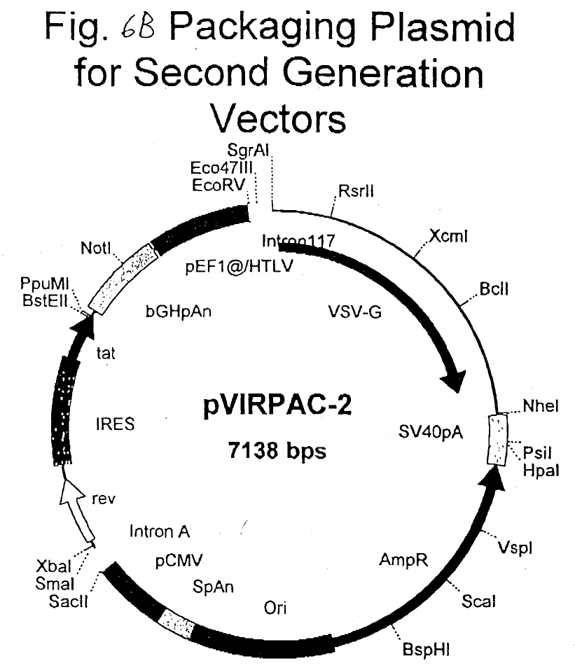

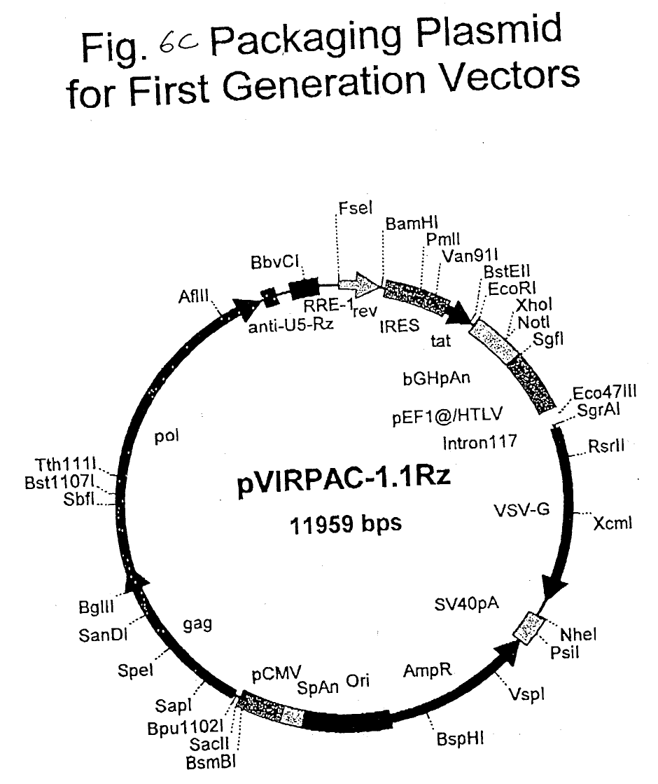

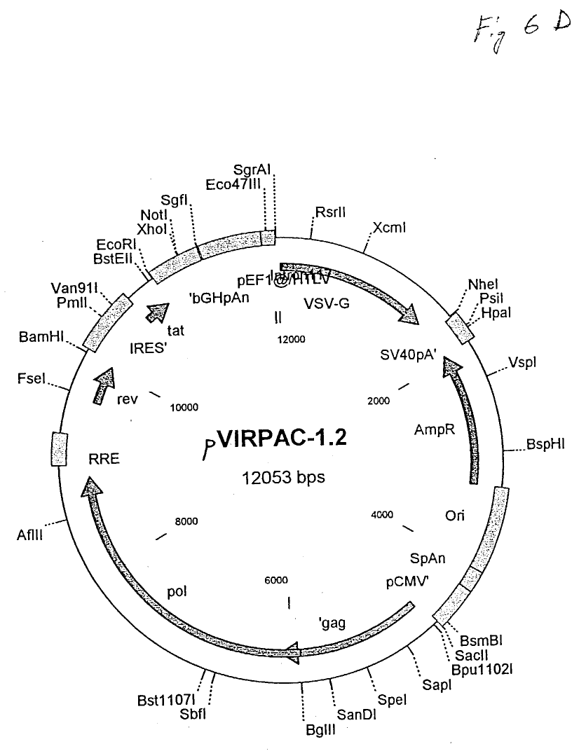

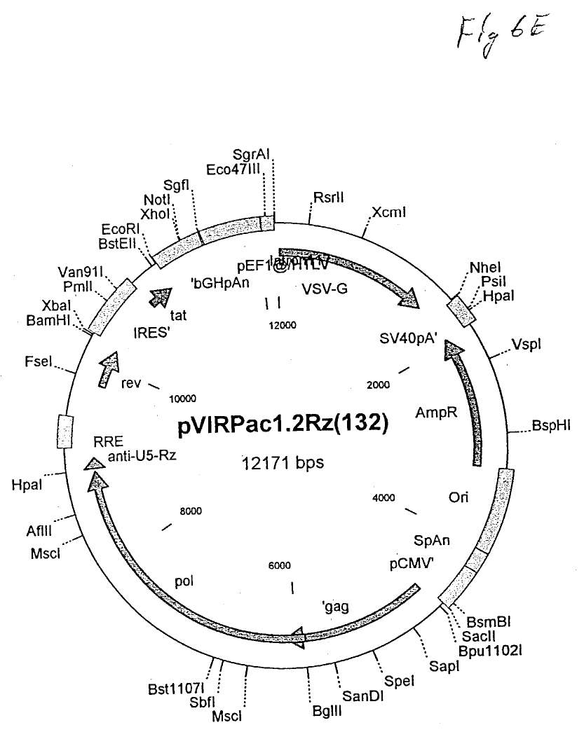

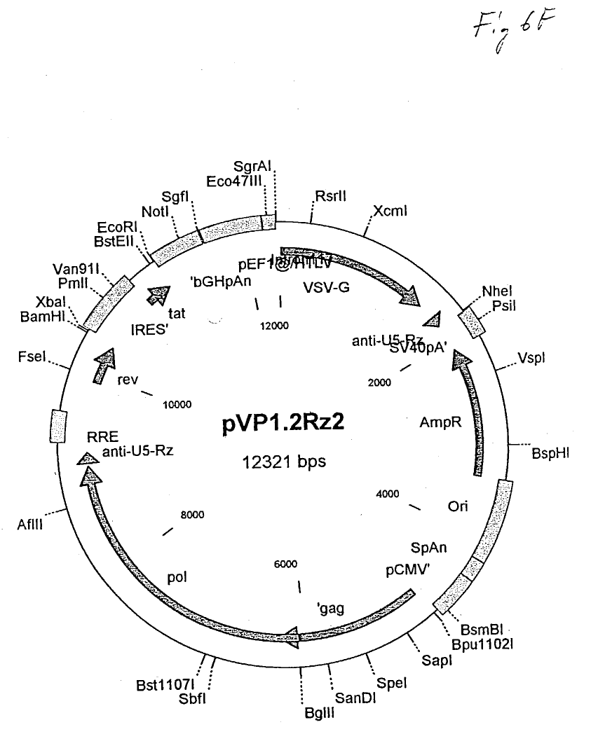

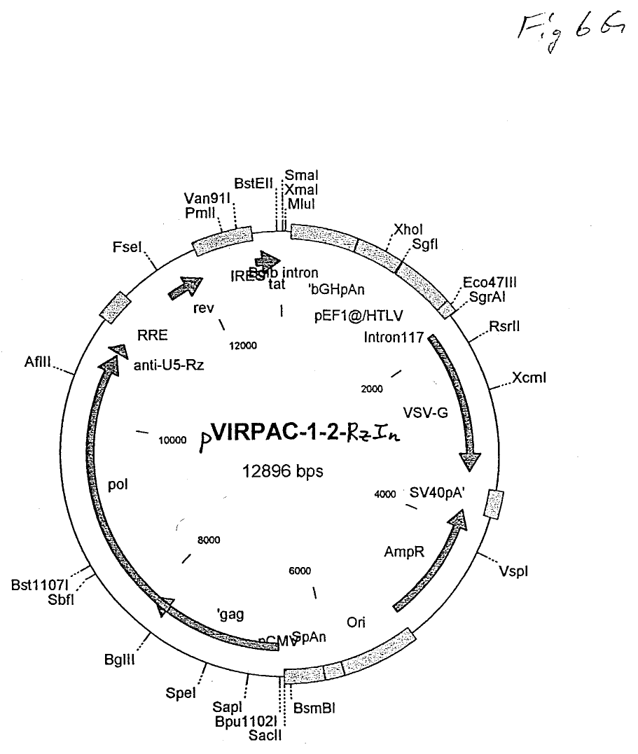

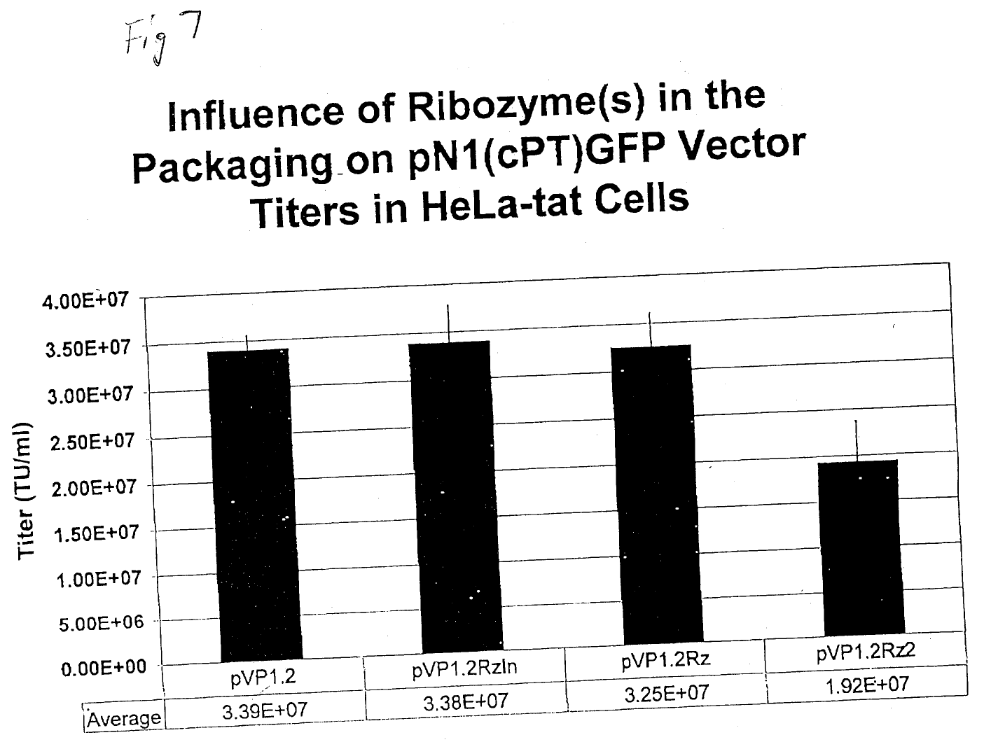

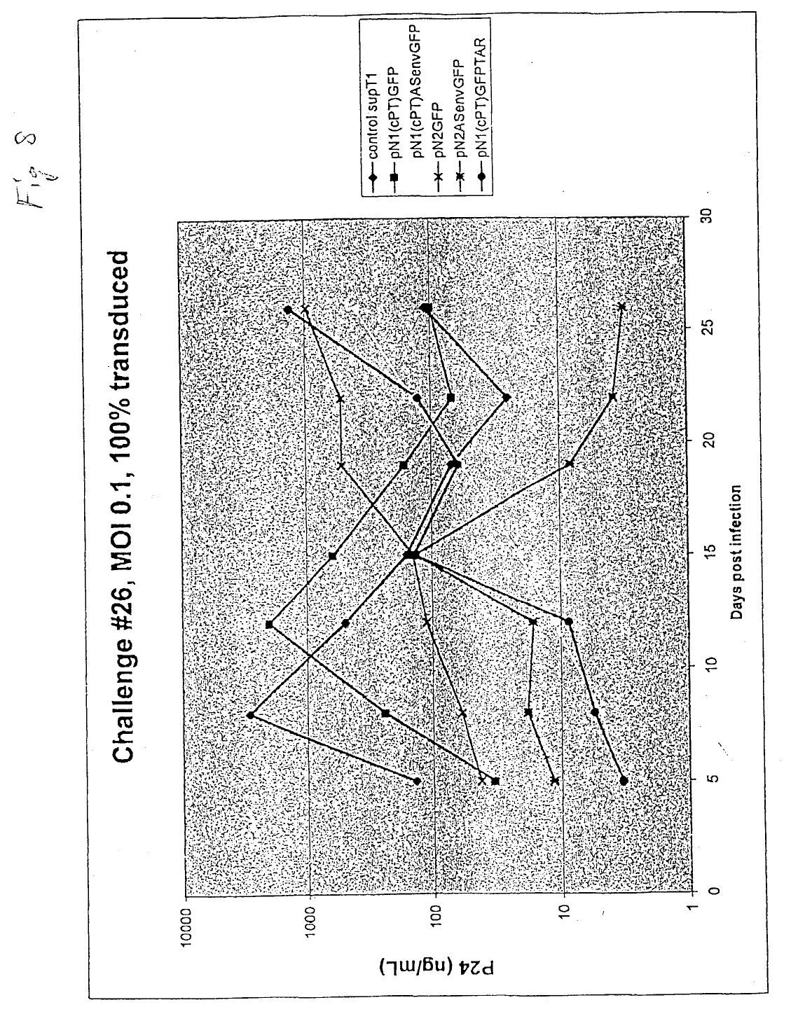

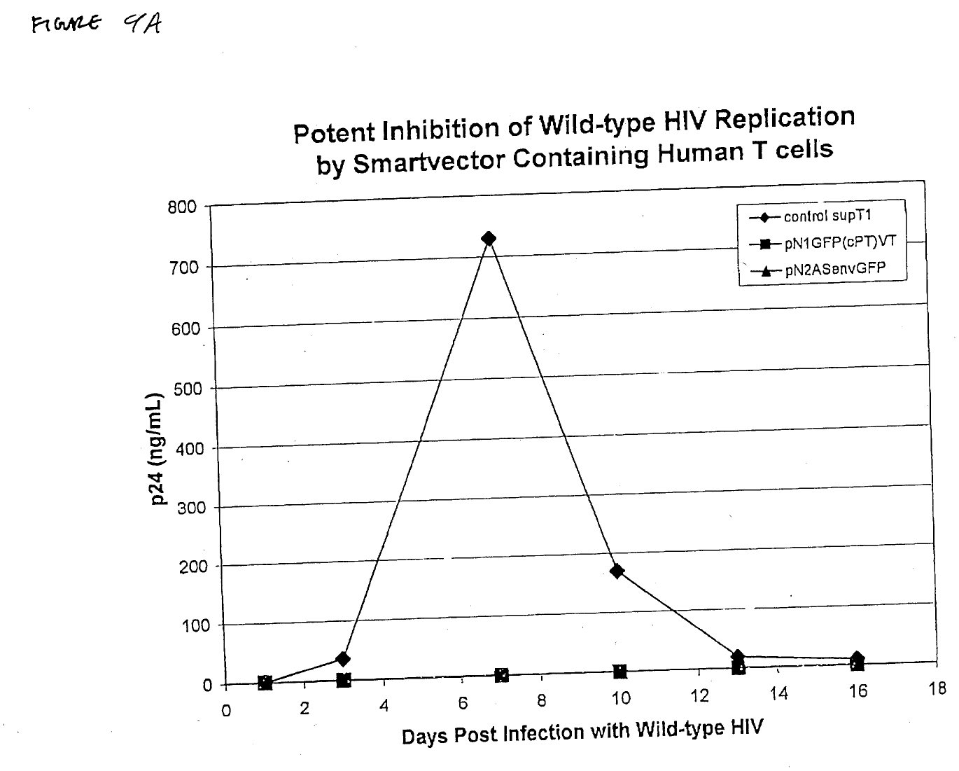

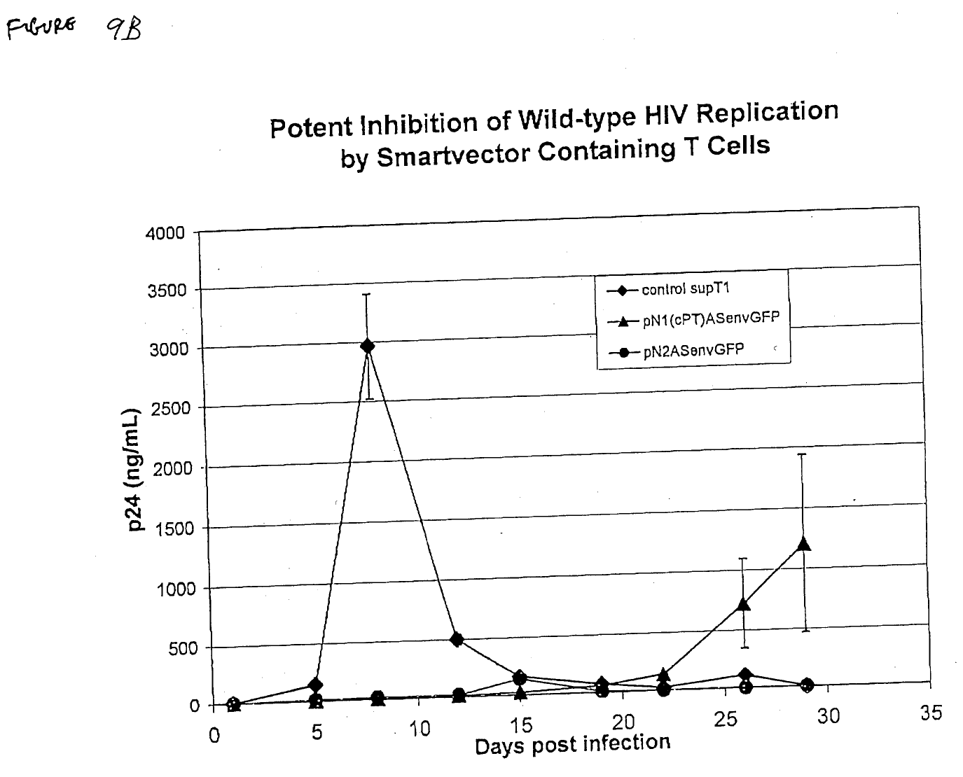

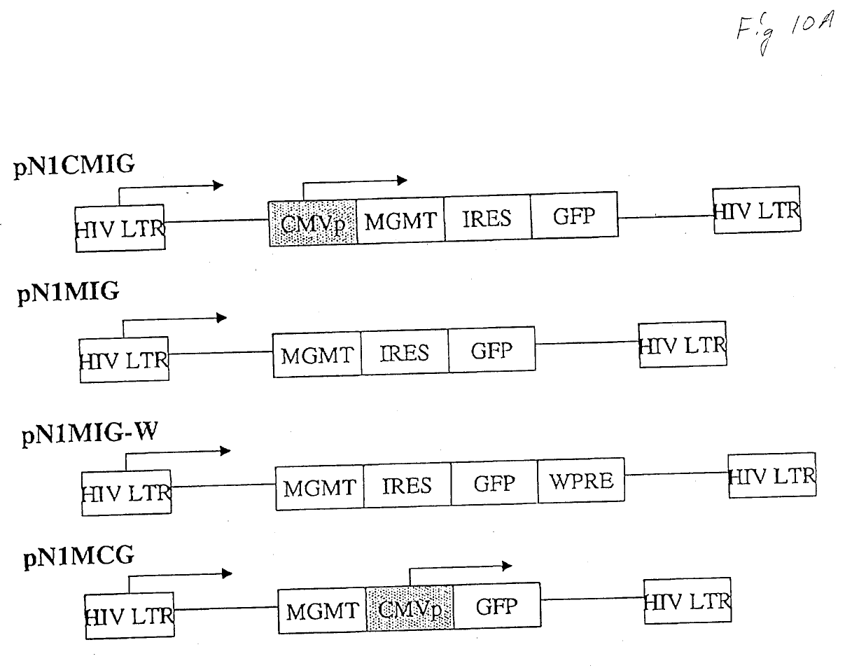

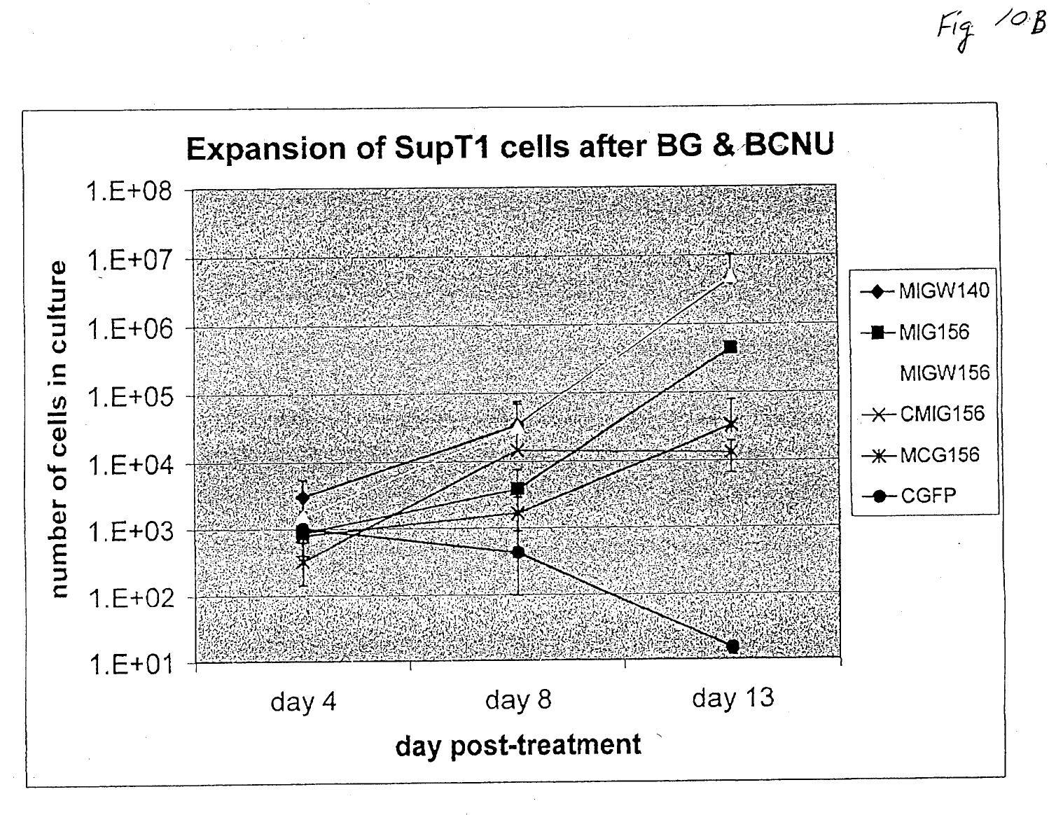

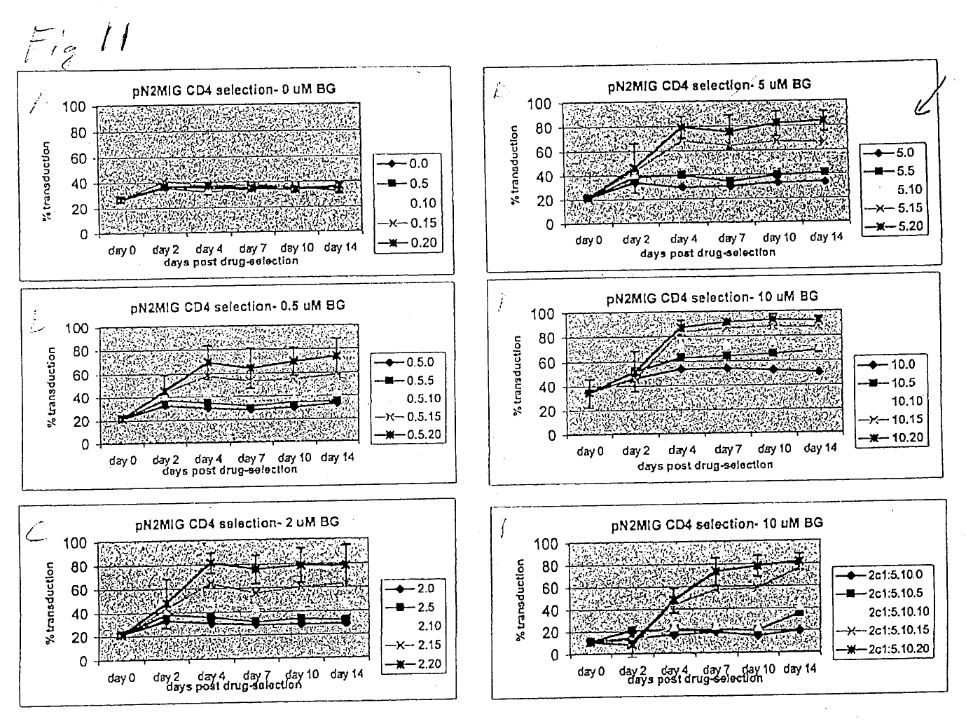

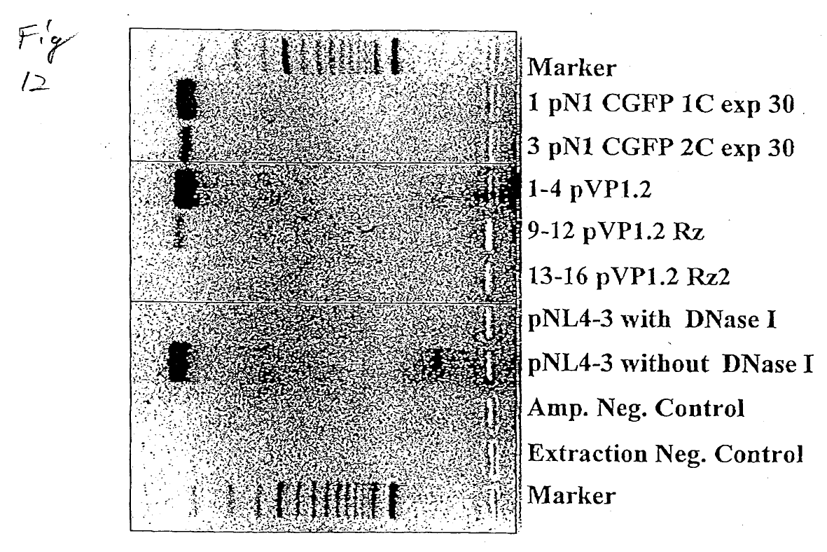

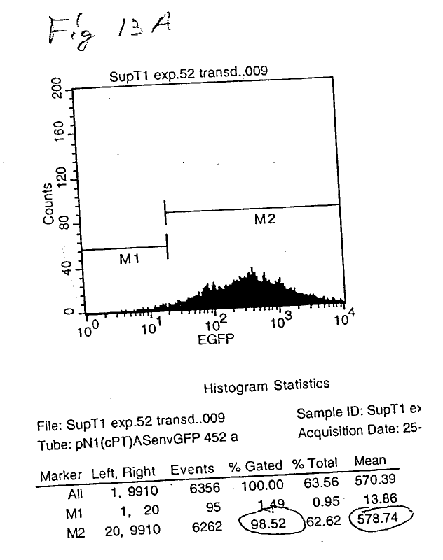

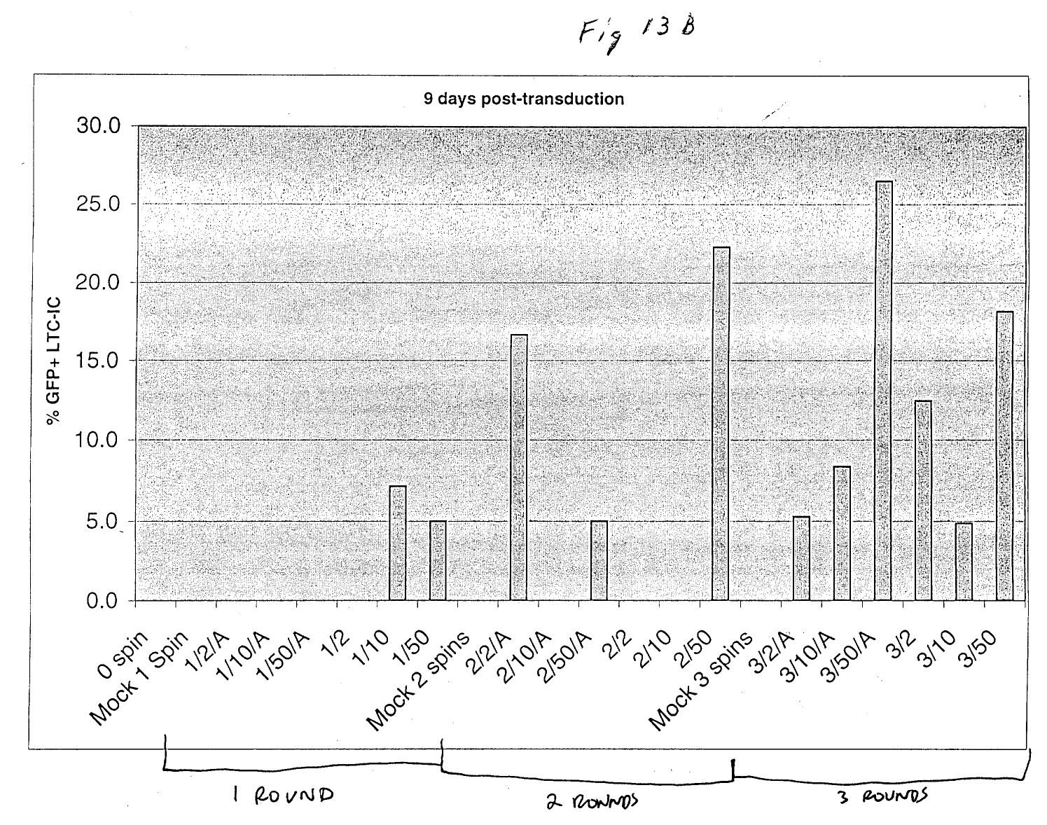

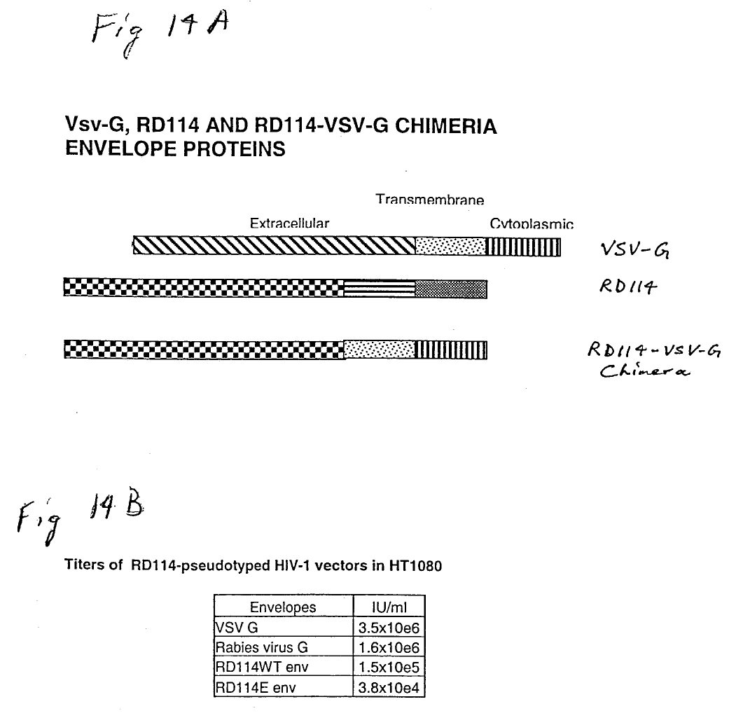

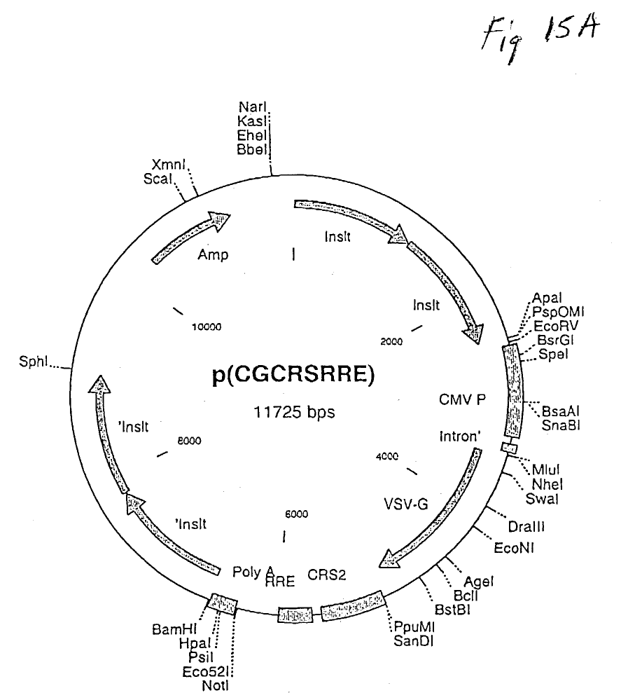

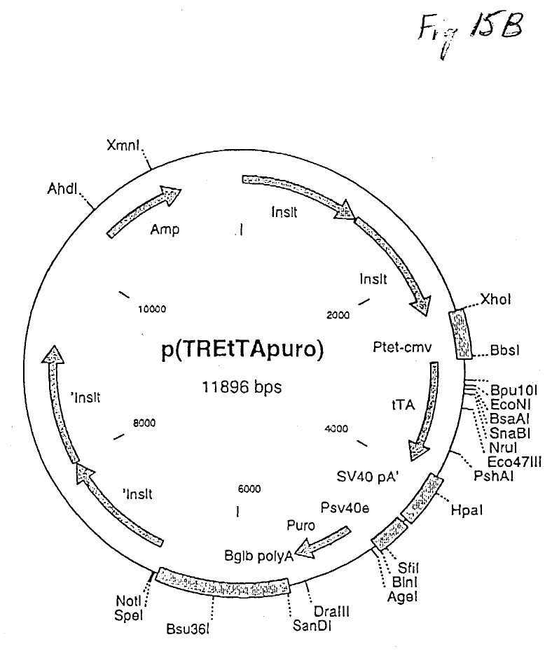

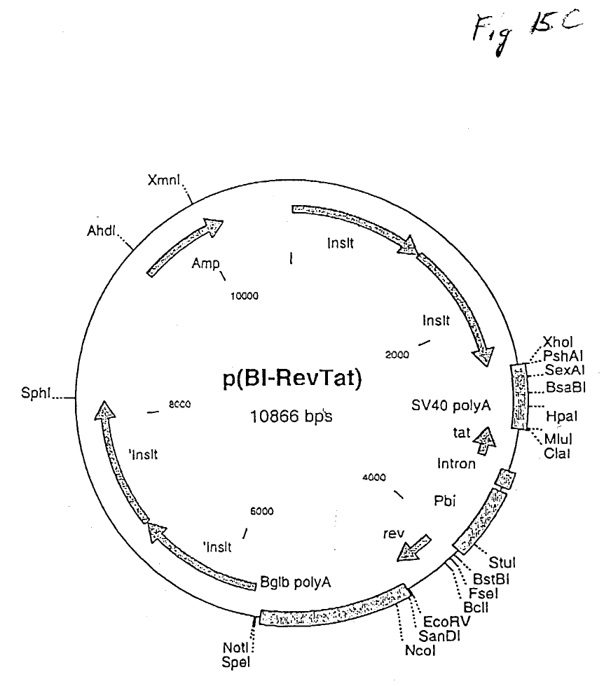

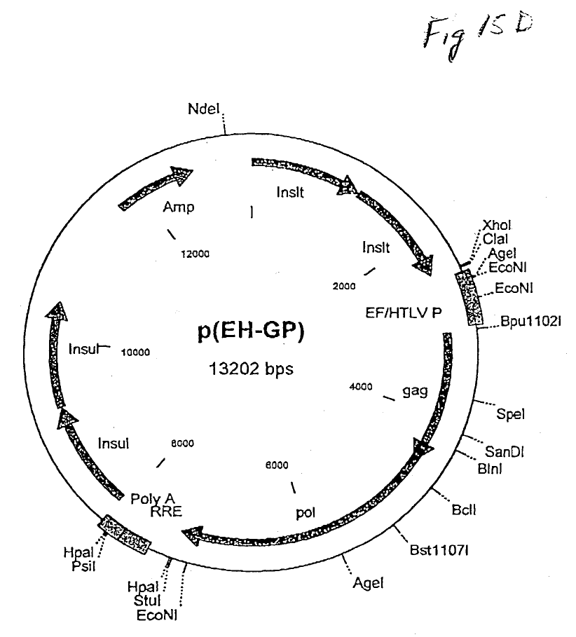

Such a ribozyme is able to cleave within the wild-type HIV genome (or its transcripts) but not the vector genome (or its transcripts) inasmuch as the vector U5 sequences are modified by in vitro site-directed mutagenesis, such as is known in the art and described in Example 1. In particular, the vector sequences preferably are modified such that the vector comprises a sequence selected from the group consisting of SEQ ID NO:2 (i.e., GTGTGCCCACCTGTTGTGTGACTCTGGCAGCTAGAGAAC), SEQ ID NO:5, (i.e., GTGTGCCCGCCTGTTGTGTGACTCTGGTAACTAGAGATC), SEQ ID NO:6 (i.e., GTGTGCCCGTCTGTTGTGTGACTCTGGCAAC TAGAGATC), SEQ ID NO:14, in which at least one N is mutated, SEQ ID NO:15 and SEQ ID NO:16. In the form of RNA, the vector preferably comprises a sequence encoded by a sequence selected from the group consisting of SEQ ID NO:5, SEQ ID NO:6, SEQ ID NO:14, in which at least one N is mutated, SEQ ID NO:15 and SEQ ID NO:16. In contrast, wild-type HIV comprises the US sequence encoded by the sequence of SEQ ID NO:1 (i.e., GTGTGCCCGTCTGTTGTGTGACTCTGGTAACTAGAGATC). The modifications in toto and comparison to the wild-type U5 sequence (in the form of DNA) are set out in FIG. 2.

Moreover, other ribozymes targeted to other regions of a viral and, particularly, a HIV genome can be employed, either alone or in combination. For instance, the ribozyme can cleave within other RNA sequences needed for viral replication, e.g., within the reverse transcriptase, protease, or transactivator protein, within Rev, or within other necessary sequences, such as have been described. Preferably, a vector comprises multiple ribozymes, e.g., targeted to multiple sites. In such cases, the analogous sequences in the vector are modified by site-directed mutagenesis, or some other means such as is known in the art, to derive a vector that is resistant to such ribozyme cleavage.

When the vector is a human immunodeficiency virus, preferably the vector lacks the tat gene and its 5′ splice site and, in place thereof, comprises a triple anti-Tat ribozyme cassette, wherein the catalytic domain of each ribozyme of the triple ribozyme cassette cleaves a different site on a wild-type human immunodeficiency viral nucleic acid molecule, in particular a different site within tat. Preferably, the catalytic domain of each ribozyme cleaves a nucleotide sequence in a region of a nucleic acid molecule of wild-type human immunodeficiency virus for which there is no ribozyme-sensitive counterpart in the vector, itself.

A further embodiment of a ribozyme as a genetic antiviral agent is available in ribozymes that target sequences not only by Watson-Crick base pairing but also by G-U wobble base pairing. Previous studies have shown that the G-U wobble in double stranded RNA has similar features as Watson-Crick base pairing. Given the high mutation rate, even in highly conserved regions, in HIV that gives rise to different HIV strains, the ability to design a ribozyme which targets multiple strains provides a dramatic advantage to the present invention. For example, a highly conserved target region in the tat gene, which may be used as a target sequence for the ribozymes of the present invention, has either a “G” or an “A” in 40 well defined HIV-1 strains. Thus the ribozyme targeting this region can be designed to contain a “U” instead of a “C” in the appropriate targeting sequence to permit pairing with either the “G” or the “A” residue. This may be compared to the use of a “C” in the targeting sequence which would reduce the ability of the ribozyme to target the HIV-1 strains having an “A” in the corresponding position. This approach of using a “U” anywhere there is a G, A variation at a target position for ribozyme recognition significantly increases the utility of the ribozymes of the invention.

A further embodiment for the practice of the disclosed invention is to use a ribozyme cassette (which contains more than one ribozyme linked in tandem) degenerated so that it is not a directly repeated sequence in the vector. The presence of degenerated sequences prevents the deletion of repeated sequences as observed in retroviral vectors and other nucleic acids. Therefore, a ribozyme cassette composed of tandemly arranged direct repeat sequences may be deleted during multiple cycles of replication. Degenerated ribozyme sequences may be used to overcome this problem because sequences in the catalytic domain of the hammerhead ribozymes molecule, for example, can be substituted with other nucleotides, based on wobble base pairing, without significant loss of activity. Mutagenesis of the hammerhead ribozymes has been previously performed and the sites that can be degenerated determined (Rufffier D E, Stormo G D, Uhlenbeck O C. Sequence requirements of the hammerhead RNA self-cleavage reaction. Biochemistry. 1990 November 27;29(47):10695-702). Another means of decreasing the likelihood of deletions is by directing the individual ribozymes of a cassette to target different sites of the target RNA. Thus the cassette would not contain the presence of direct repeat sequences.

A “transdominant” or “dominant negative” nucleic acid sequence confers its encoded phenotype even in the presence of a wildtype (wt) sequence. The discussion of the truncated CCR5 protein (above) is one example. Other examples include any mutant retroviral or lentiviral sequence that confers a transdominant phenotype. Examples include the rev and gag genes.

One example for use in providing a selective advantage as well as the inhibition of an infectious wildtype virus is with transdominant gag sequences that have been shown to inhibit HIV-1 replication, possibly by interfering with capsid assembly. Thus such a sequence is preferably used in combination with a non-HIV-1 based retroviral vector to permit its replication and packaging without inhibition from the transdominant sequence. For example, a retroviral vector containing HIV-2 gag and pol genes may include a transdominant HIV-1 gag sequence. Such a vector may be replicated and packaged via complementation with an appropriate HIV-2 helper vector construct. But after deployment in a cell subject to HIV-1 infection, the transdominant gag sequence is available for expression upon HIV-1 infection to block mobilization of the HIV-1 virus. The retroviral vector encoded HIV-2 gag and pol genes are still available, however, to encapsidate and mobilize the vector.

2. Nucleic acid sequence, the possession of which confers a selective advantage to cells infected, or uninfected, with a vector comprising the sequence as compared with cells infected with a wild-type strain of virus.

A nucleic acid sequence that confers a selective advantage to a cell containing a vector comprising the sequence over a cell containing, or not containing, a wild-type strain of virus (i.e., that lacks the sequence) preferably is any sequence that allows a cell containing the vector to survive and propagate viral particles (i.e., crHIV viral particles) as compared with a cell containing the wild-type virus, or with a cell absent the nucleic acid sequence. Such sequences include, but are not limited to, any sequence that allows the cell, or the vector contained in the cell, to escape destruction, sequences that promote cell survival, sequences that induce apoptosis, sequences that facilitate protein production or sequences that promote immune function or targeting.

For instance, preferably such a nucleic acid sequence contained on the vector encodes genes for multidrug resistance (see., e.g., Ueda et al., Biochem. Biophys. Res. Commun., 141, 956-962 (1986); Ueda et al., J. Biol. Chem., 262, 505-508 (1987); and Ueda et al., PNAS, 84, 3004-3008 (1987)). In the presence of added cytotoxic drug (e.g., as used for cancer chemotherapy), this allows a cell containing the vector to survive, whereas a cell that contains wild-type virus, such as HIV, does not. Such cytotoxic drugs include, but are not limited to, actinomycin D, vinblastine sulfate, vincristine sulfate, BCNU (with or without BG conditioning), daunomycin, adriamycin, VP-16, and AMSA.

Another example of such a nucleic acid sequence is any sequence variant of the O⁶-methylguanine-DNA-methyltransferase (MGMT). MGMT may be used to protect hematopoietic progenitors from the toxicity of alkylating agents. Wildtype MGMT is inhibited by O⁶-benzylguanine (BG), which potentiates alkylating agent toxicity. MGMT variants that are resistant to BG mediated inactivation and able to protect against alkylating agents may be provided to any cell type by the present invention. An example of such a variant is G156A MGMT.

For example, hematopoietic progenitor cells transduced with such a variant via the present invention may be selected for upon administration of BG and an O⁶-guanine methylating or chloroethylating agent. Examples of such methylating agents for use in the invention include, but are not limited to the clinically relevant temozolomide, nitrosoureas, tetrazines, triazines, dacabazine, temozolomide, streptozotocin, procarbazine. Examples of chloroethylating agents include, but are not limited to, BCNU (1,3-bis(2-chloroethyl)-1-nitrosourea), CCNU (3cyclohexyl-1-chloroethyl-nitrosourea), ACNU (1-(4-amino-2-methyl-5-pyrimidinyl)methyl-3-(2chloro)-3-nitrosourea), and MeCCNU (1-(2-chloroethyl)-3-(4-methylcyclohexyl)-1-nitrosourea. Preferably, the alkylating agent is BCNU.

BCNU has been shown to form a permanent, covalent interstrand DNA crosslink lesion in both quiescent and cycling cells. These lesions are cytotoxic to cells during DNA replication. MGMT is the primary mechanism of DNA repair of BCNU lesions. Given its toxicity to quiescent cells, selection with MGMT variants delivered by lentiviral vectors of the invention can also permit the selection for totipotent hematopoietic stem cells (HSCs), which are predominantly in G₀ of the cell cycle, but which cycle intermittently for production of more stem cells and blood progenitor cells. Stem cells are generally resistant to retroviral transduction but can be transduced with retroviral vectors if they are not solely in a Go cycle state. Like onco-retroviruses, the lentiviral vectors of the invention can be used to transduce hematopoietic stem cells, including assayable hematopoietic ELTC-IC, and NOD-SCID or SCID-hu repopulating cells. However, unlike the onco-retroviruses, the cytokine conditions required for transducing stem cells without their differentiation is likely to favor lentiviral vectors since lentiviruses (e.g. HIV) can infect cells that are not actively dividing at the time of infection. HSC selection is not possible with other strategies that select for cycling hematopoietic progenitors. Thus selection mediated by MGMT variants and the lentiviral vectors of the invention can increase the presence of transduced totipotent HSCs without the need for prolonged drug administration for selection.

The above discussion may be viewed in the context of hematopoietic cells in vitro and ex vivo as well as in vivo. Thus in addition to transducing cells in vivo with MGMT variants, hematopoietic cells of a subject may be transduced ex vivo followed by drug treatment either ex vivo or in vivo. Such ex vivo treated cells may then be infused into a subject, optionally as part of repopulating the bone marrow with transduced cells.

Before the implementation of the above, the alkylating agent BCNU can also be used to generally reduce HIV infected CD4+ cells in HIV positive subjects. As stated above, BCNU (after depletion of endogenous MGMT) forms cytotoxic lesions in both quiescent and proliferating cells. Such lesions form even without forced depletion of MGMT if the drug dose overwhelms the level of functional AGT protein. Repetitive systemic treatment with BCNU causes cumulative myelosuppression and pancytopenia. Among lymphoid cells, CD4+ cells appear to be remarkably sensitive to BCNU. Thus in retroviral infected subjects, such as HIV infected patients, BCNU administration, including in low doses to avoid myelosuppresssion if desired, will decrease the total CD4+ cell population and so reduce the viral load. This approach may be followed by infusion of hematopoeitic progenitors as discussed above.

Because BCNU may act in a “stealth” manner by forming cytotoxic lesions even in resting cells, this approach has the added advantage of reducing the need for repeated treatments. Of course this approach can be combined with the use of BG to potentiate the effects of BCNU. Alternatively, the approach can be used in combination with the introduction of MGMT variants with the vectors of the invention to provide protection for transduced CD4+ cells. This latter means can be either independent of or in conjunction with the treatment of hematopoeitic cells as discussed above. Therefore, a preferred embodiment of the invention is to express mutant MGMT (a second nucleic acid sequence) in a vector that contains an anti-HIV antisense or ribozyme (a first nucleic acid sequence) so that the vector provides both a selective advantage over wild-type HIV infected cells and a selective advantage over wild-type HIV virus, respectively. A further preferred embodiment is that the vector expresses the MGMT gene (or second nucleic acid sequence) off the HIV-LTR promoter, as a spliced mRNA.

Finally, although the vectors express anti-HIV ribozymes or antisense sequences, the invention is not so limited since anyone skilled in the art may readily insert any inhibitory nucleotide sequence into a vector for a particular therapeutic, prophylactic or biological effect. A preferred method to express the inhibitory sequence is to include it in a U1 snRNA/promoter cassette as described in Dietz (U.S. Pat. No. 5,814,500).

Alternatively, such a nucleic acid sequence desirably comprises a sequence selected from the group consisting of a sequence of (or a sequence that encodes) a mutated (i.e., mutant) protease, and a sequence of (or a sequence that encodes) a mutated (i.e., mutant) reverse transcriptase. Preferably, a mutated reverse transcriptase is engineered to be resistant to nucleoside and non-nucleoside reverse transcriptase inhibitors, and a mutated protease is engineered to be resistant to commonly employed protease inhibitors.

Administration of these protease or reverse transcriptase inhibitors to a host in conjunction with the vector is employed to select for cells producing the vector as opposed to cells producing the wild-type virus. Similarly, this approach is modified for use with any drug that inhibits viral replication such that the virus can be mutated to escape from inhibition. Accordingly, for treatment of HIV, the selective nucleic acid sequence incorporated into the vector preferably comprises mutated HIV sequences. Optimally, however, these sequences do not prevent superinfection with wild-type HIV.

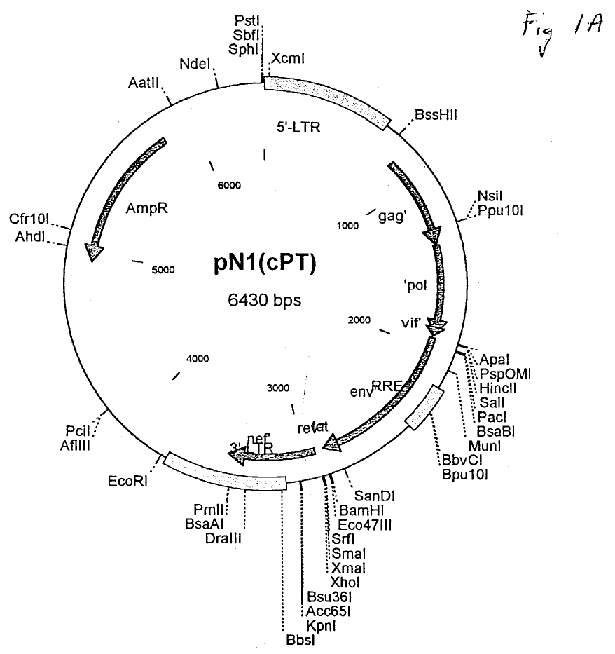

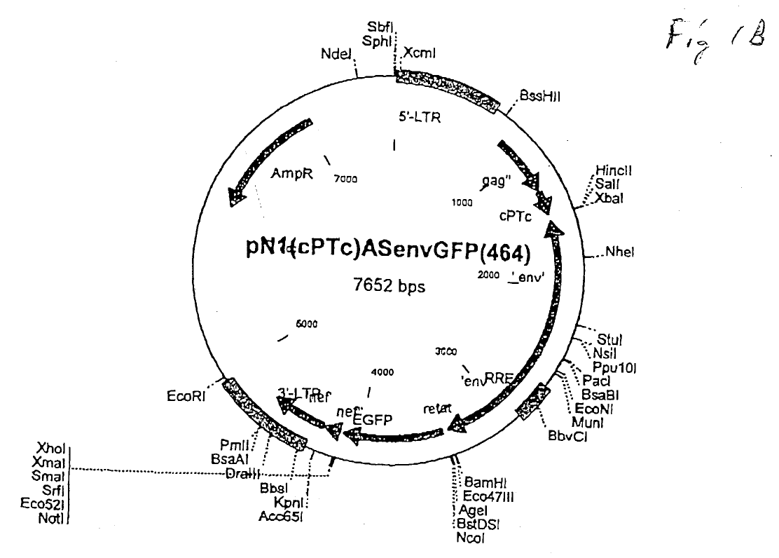

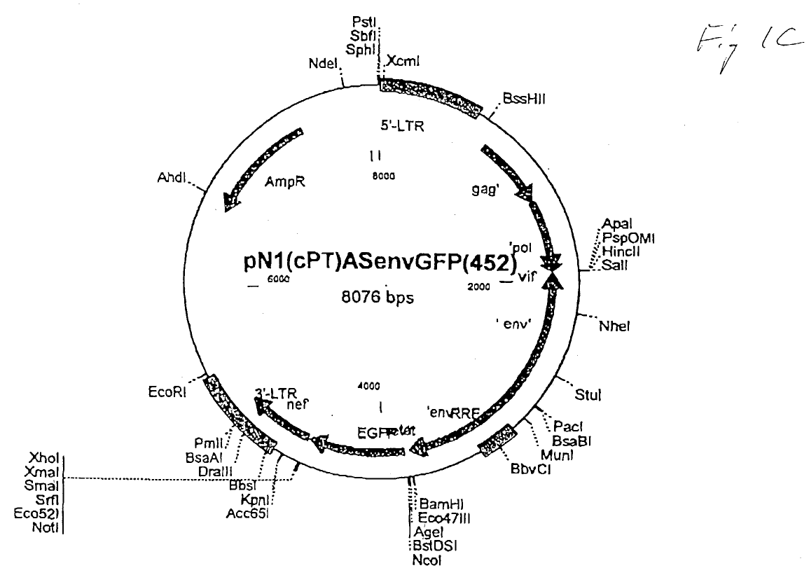

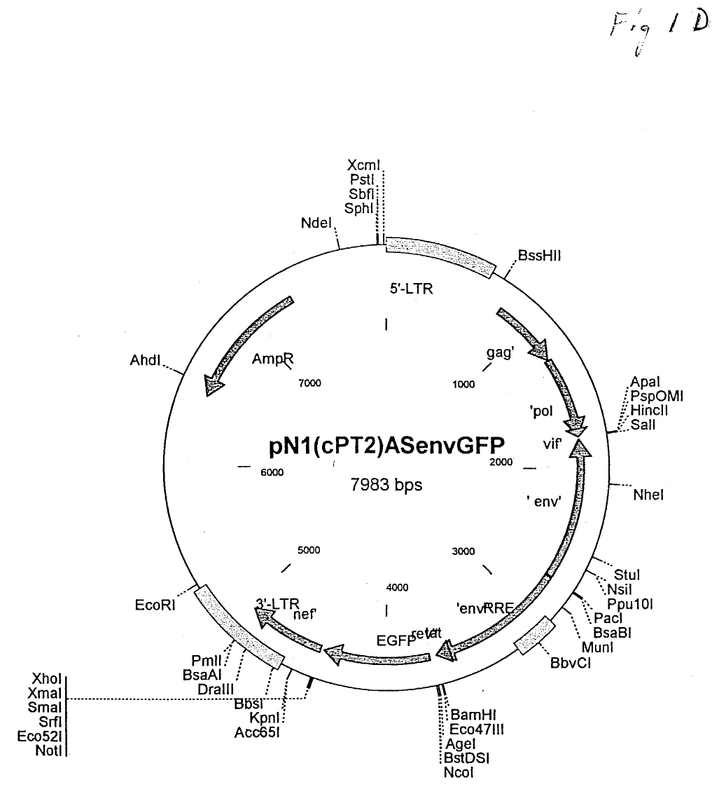

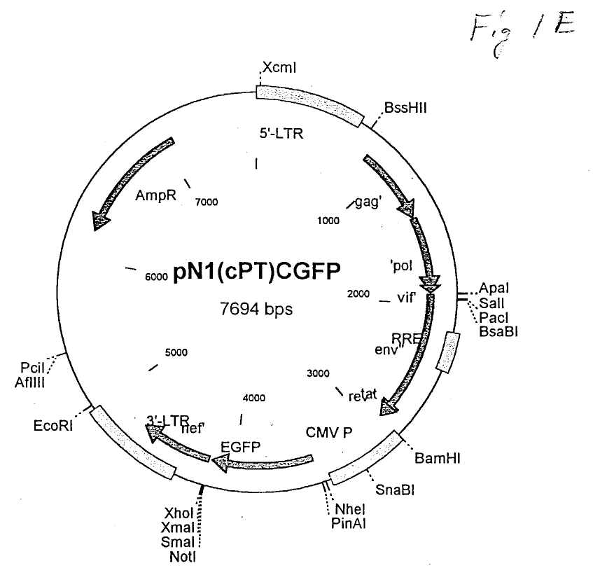

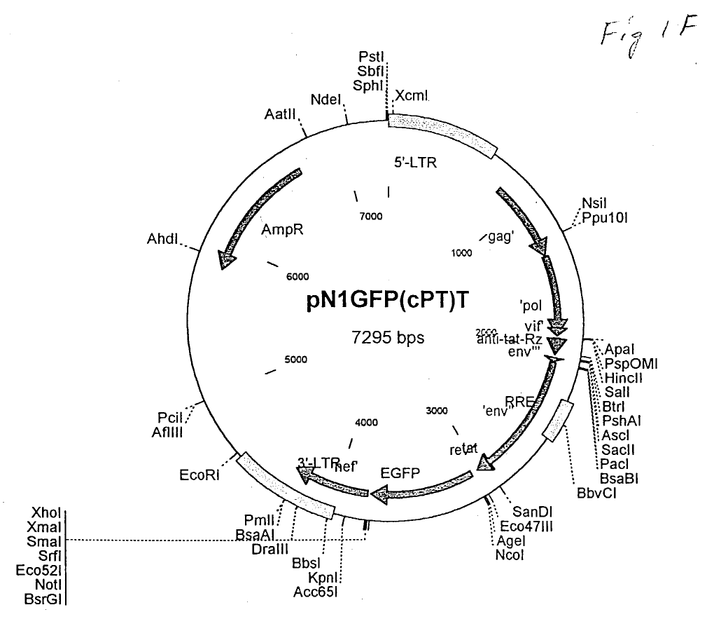

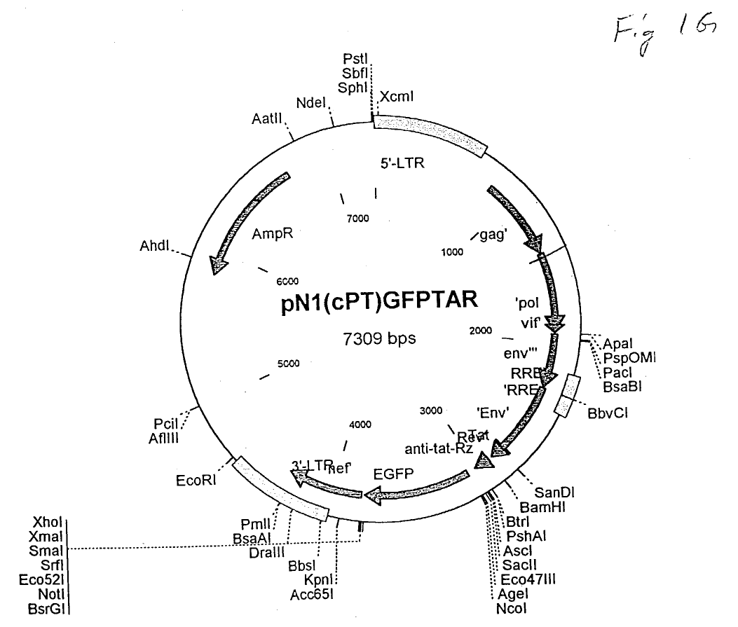

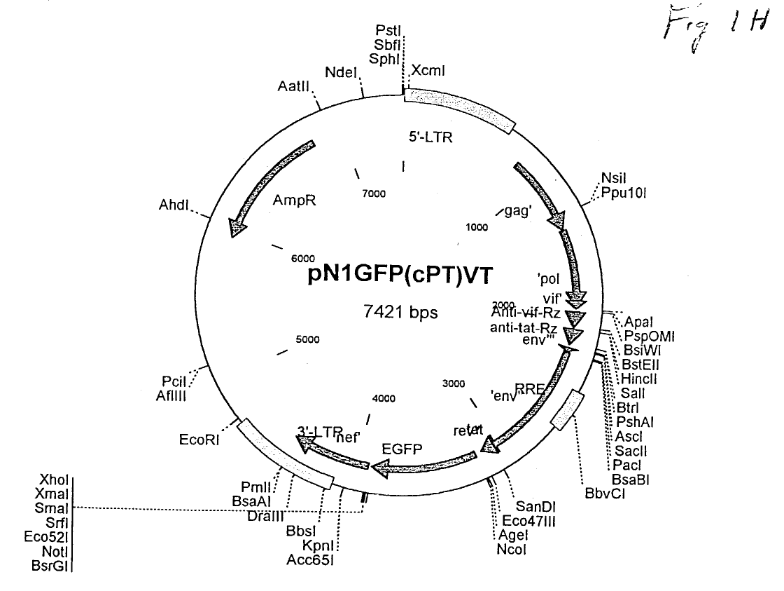

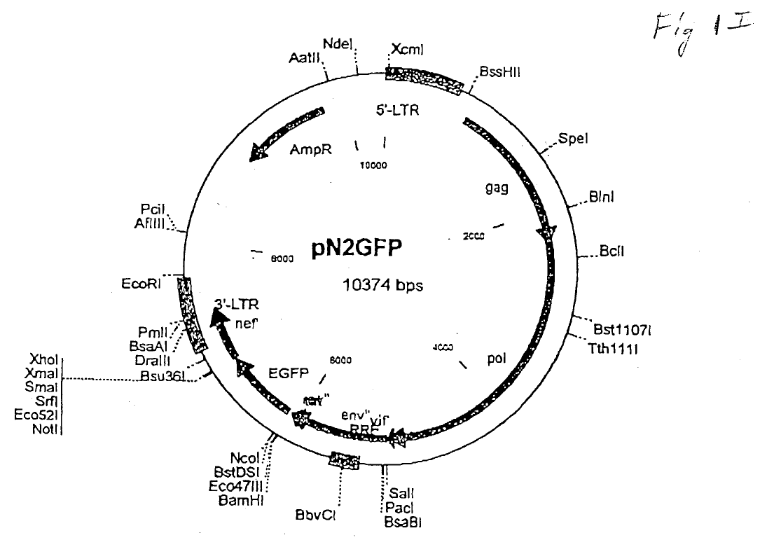

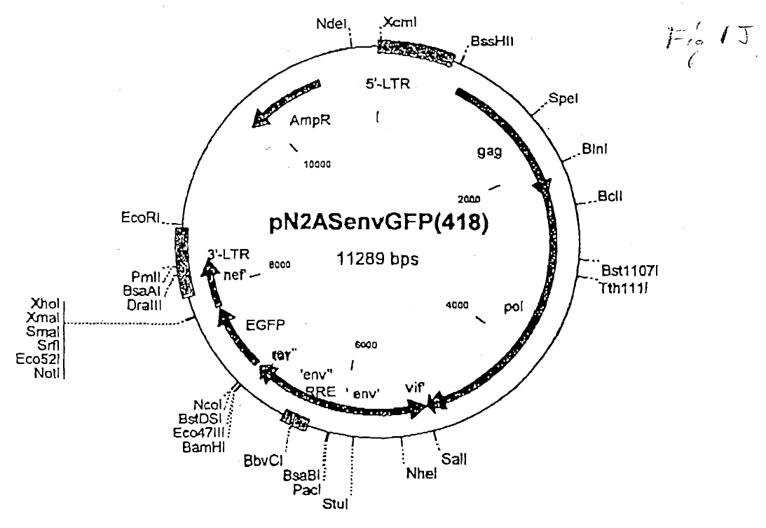

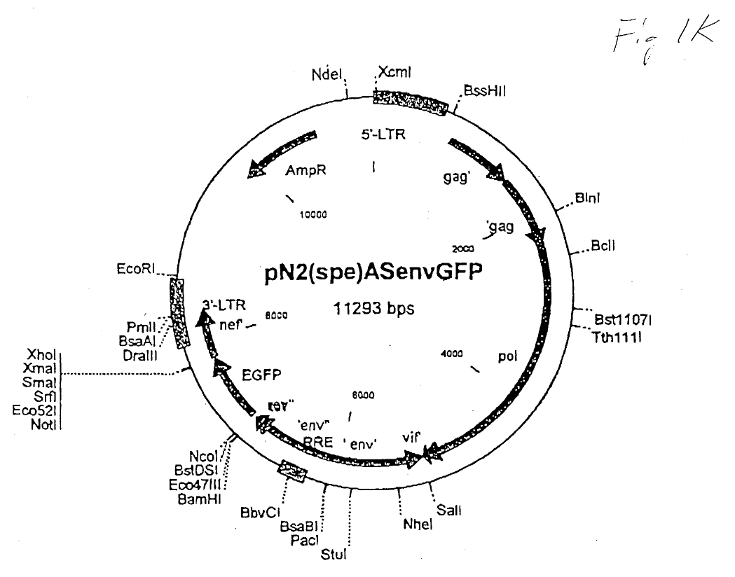

Preferably, the vector is one of those set forth above, and, in particular, the improved conditionally replicating vectors depicted in FIGS. 1A-1K. Of course when used in methods of the invention, the GFP encoding sequences may be deleted or replaced with other sequences. The GFP encoding sequences are present in these exemplary vector embodiments as a marker or indicator of the presence of the vector or gene expression from the vector.

The preferred vectors of the invention may contain the various elements described in the present disclosure. In paticular, the lentiviral vectors may lack the tat gene and contain at least one antisense sequence. Additionally, the vectors may contain a sequence such as the central polypyrimidine tract of HIV or a larger nucleic acid fragment containing it.

Optimally, a vector is compatible with the cell into which it is introduced, e.g., is capable of imparting expression on the cell of the vector-encoded nucleic acid sequences. Desirably, the vector comprises an origin of replication functional in the cell. When a nucleic acid sequence is transferred in the form of its DNA coding sequence (e.g., versus in the form of a complete gene comprising its own promoter), optimally the vector also contains a promoter that is capable of driving expression of the coding sequence and that is operably linked to the coding sequence. A coding sequence is “operably linked” to a promoter (e.g., when both the coding sequence and the promoter together constitute a native or recombinant gene) when the promoter is capable of directing transcription of the coding sequence.

In a recombinant vector of the present invention, preferably all the proper transcription (e.g., initiation and termination signals), translation (e.g., ribosome entry or binding site and the like), processing signals (e.g., splice donor or acceptor sites, if necessary, and polyadenylation signals), translocation, assembly, integration sites, ribonuclear complex entry, stability and translocation elements, in cis or trans, are arranged correctly on the vector, such that any gene or coding sequence is appropriately transcribed (and/or translated, if so desired) in the cells into which the vector is introduced. The manipulation of such signals to ensure appropriate expression in host cells is well within the knowledge and expertise of the ordinary skilled artisan.

Preferably the vector contains a Pol sequence that increases the efficiency of stable transduction, defined as integrated copies of the vector in the host cell genome. A previously identified sequence by Zennou et al. (Cell 101:173-185 (2000)) as a central DNA flap (a 178 base pair fragment from positions 4793 to 4971 on pLAI3, corresponding to positions 4757 to 4935 on pNL4-3) was reported to increase transduction efficiency was found to be insufficient to significantly increase the efficiency of stable transduction. The present invention includes the discovery that while this small fragment is not sufficient to increase the efficiency of stable transduction, a larger 545 base pair fragment (positions 4551 to 5096 in pNL4-3), or yet larger fragments containing it, as described in U.S. Pat. No. 5,885,806 was capable of increasing stable transduction as part of the present invention. The increase in stable transduction efficiency was detected by GFP expression and FACS analysis, and Taqman analysis of integrated copies of vector genome.

Additional means of increasing transduction efficiency are described in co-pending U.S. patent application Ser. No. ______ (yet to be assigned) filed Aug. 31, 2000 as attorney docket no. 397272000400, which is hereby incorporated by reference as if fully set forth.

The viral vectors used in the present invention may also result from “pseudotype” formation, where co-infection of a cell by different viruses produces progeny virions containing the genome of one virus encapsulated within an outer layer containing one or more envelope protein of another virus. This phenomenon has been used to package viral vectors of interest in a “pseudotyped” virion by co-transfecting or co-infecting a packaging cell with both the viral vector of interest and genetic material encoding at least one envelope protein of another virus or a cell surface molecule. See U.S. Pat. No. 5,512,421. Such mixed viruses can be neutralized by anti-sera against the one or more heterologous envelope proteins used. One virus commonly used in pseudotype formation is the vesicular stomatitis virus (VSV), which is a rhabdovirus. The use of pseudotyping broadens the host cell range of the virus by including elements of the viral entry mechanism of the heterologous virus used. Pseudotyping of viral vectors and VSV for use in the present invention results in viral particles containing the viral vector nucleic acid encapsulated in a nucleocapsid which is surrounded by a membrane containing the VSV G protein. The nucleocapsid preferably contains proteins normally associated with the viral vector. The surrounding VSV G protein containing membrane forms part of the viral particle upon its egress from the cell used to package the viral vector. Examples of packaging cells are described in U.S. Pat. No. 5,739,018. In a preferred embodiment of the invention, the viral particle is derived from HIV and pseudotyped with VSV G protein. Pseudotyped viral particles containing the VSV G protein can infect a diverse array of cell types with higher efficiency than amphotropic viral vectors. The range of host cells include both mammalian and non-mammalian species, such as humans, rodents, fish, amphibians and insects.

Preferably, the vector also comprises some means by which the vector or its contained subcloned sequence is identified and selected. Vector identification and/or selection is accomplished using a variety of approaches known to those skilled in the art. For instance, vectors containing particular genes or coding sequences preferably are identified by hybridization, the presence or absence of so-called “marker” gene functions encoded by marker genes present on the vectors, and/or the expression of particular sequences. In the first approach, the presence of a particular sequence in a vector is detected by hybridization (e.g., by DNA-DNA hybridization) using probes comprising sequences that are homologous to the relevant sequence. In the second approach, the recombinant vector/host system is identified and selected based upon the presence or absence of certain marker gene functions such as resistance to antibiotics, thymidine kinase activity, and the like, caused by particular genes encoding these functions present on the vector. In the third approach, vectors are identified by assaying for a particular gene product encoded by the vector. Such assays are based on the physical, immunological, or functional properties of the gene product.

Accordingly, the present invention also provides a vector, which, if DNA, comprises a nucleotide sequence selected from the group consisting of SEQ ID NOS:2, 4, 5, 6, 7, 15, 16, 17 and 18 and, which, if RNA, comprises a nucleotide sequence encoded by a nucleotide sequence selected from the group consisting of SEQ ID NOS:4, 5, 6, 7.

The present invention further provides a method of engendering a vector, which is derived from a wild-type human immunodeficiency virus and which is capable of replicating only in a host cell that is permissive for replication of said vector, with a ribozyme. The ribozyme, which is comprised within or encoded by the vector, cleaves a nucleic acid of a wildtype human immunodeficiency virus but not the vector, itself, and its transcripts, if any. The method comprises obtaining a vector, which is derived from a wild-type human immunodeficiency virus and which is capable of replicating only in a host cell that is permissive for replication of said vector, and incorporating into the vector a nucleic acid sequence, which comprises or encodes a ribozyme, the catalytic domain of which cleaves a nucleic acid of a wildtype human immunodeficiency virus but not the vector, itself, and its transcripts, if any. In such a method, the nucleotide sequence comprising or encoding the U5 sequence of the wild-type human immunodeficiency virus can be deleted from the vector and replaced with a nucleotide sequence selected from the group consisting of SEQ ID NOS:2, 5, 6, 14, in which at least one N is mutated, 15 and 16 if the vector is DNA, and a nucleotide sequence encoded by a nucleotide sequence selected from the group consisting of SEQ ID NOS:2, 5, 6, 14, in which at least one N is mutated, 15 and 16, if the vector is RNA. Preferably, the vector replicates in a host cell permissive for replication of said vector more than once.

Also provided by the present invention is a method of modifying a vector. The method comprises obtaining a vector and introducing into the vector a nucleotide sequence selected from the group consisting of the DNA sequences of SEQ ID NOS:2, 2, 3, 4, 5, 6, 14, in which at least one N is mutated, 15 and 16, if the vector is DNA, and a nucleotide sequence encoded by a nucleotide sequence selected from the group consisting of SEQ ID NOS:2, 4, 5, 6, 7, 15, 16, 17 and 18, if the vector is RNA.

Further provided by the present invention is a method of propagating and selectively packaging a conditionally replicating vector without using a packaging cell line. The method comprises contacting the conditionally replicating vector with a cell capable of being infected by another vector, which is the same type of vector as the conditionally replicating vector and which differs from the conditionally replicating vector by being wild-type for replication competency; subsequently contacting the cell with the other vector; and then culturing the cell under conditions conducive to the propagation of the conditionally replicating vector. The helper vector discussed above and in more detail below is one such complementary vector.

Also provided is an isolated and purified nucleic acid molecule selected from the group consisting of a DNA molecule comprising a nucleotide sequence selected from the group consisting of SEQ ID NOS:2, 5, 6, 14, in which at least one N is mutated, 15 and 16 and a RNA molecule comprising a nucleotide sequence encoded by a nucleotide sequence selected from the group consisting of SEQ ID NOS:2, 6, 7, 15, 16, 17 and 18.

Method of Use

The above-described vectors preferably are introduced into a host cell for the prophylactic and therapeutic treatment of viral infection, for ease of vector maintenance, as well as for other reasons. Accordingly, the present invention provides a host cell comprising a vector according to the invention. The isolation of host cells, and/or the maintenance of such cells or cell lines derived therefrom in culture, has become a routine matter, and one in which the ordinary skilled artisan is well-versed.

In particular, a conditionally replicating viral vector, or preferably a lentiviral vector, as described above preferably is employed in the prophylactic and therapeutic treatment of a viral infection, preferably such as where the infection is from a wild-type virus, preferably a wild-type RNA virus, even more preferably, from a wild-type retrovirus, and optimally from a wild-type HIV.

The method comprises contacting a host cell, which is capable of being infected with a wild-type virus, with a conditionally replicating vector, which is capable of being replicated only in a host cell permissive for the replication of the vector, the presence, transcription or translation of which inhibits the replication of the wild-type strain of virus in the host cell. Desirably, the vector replicates more than once and comprises at least one nucleic acid sequence, the possession (i.e., presence, transcription or translation) of which confers a selective advantage in a host cell to the vector over a wild-type strain of virus, which, optimally, is the strain from which the vector was derived.

According to this method, the nucleic acid sequence preferably comprises a nucleotide sequence, which comprises or encodes a genetic antiviral agent, which adversely affects the replication and/or expression of a virus other than said vector. Desirably, the genetic antiviral agent is selected from the group consisting of an antisense molecule, a ribozyme, and an immunogen. Optimally, the genetic antiviral agent is a ribozyme, preferably the catalytic domain of which cleaves at a 3′ nucleotide NUH sequence (i.e., especially a GUC sequence). Optionally, the ribozyme is encoded, at least in part, by a sequence selected from the group consisting of SEQ ID NO:3 and SEQ ID NO:4. Desirably, the ribozyme cleaves in a region of the wild-type strain of virus or its transcripts, but does not cleave in a region of the vector or its transcripts. Preferably, this is because the wild-type strain of virus comprises a sequence encoded by SEQ ID NO:1, whereas the vector, if DNA, comprises a nucleotide sequence selected from the group consisting of SEQ ID NOS:2, 5, 6, 14, in which at least one N is mutated, 15 and 16, and, if RNA, comprises a nucleotide sequence encoded by a nucleotide sequence selected from the group consisting of SEQ ID NOS:2, 5, 6, 14, in which at least one N is mutated, 15 and 16.

The method also desirably is carried out wherein the vector comprises at least one nucleic acid sequence, the possession (i.e., presence, transcription or translation) of which confers a selective advantage to a host cell infected with the vector over a cell infected with a wild-type strain of virus, which, optimally, is the strain of virus from which the vector was derived. In this regard, a vector can comprise at least one nucleic acid sequence, which confers a selective advantage to a host cell infected with the virus and at least nucleic acid sequence, which confers a selective advantage to the vector over a wild-type strain of a virus corresponding to the virus from which the vector was derived.

Accordingly, the method preferably is carried out wherein the nucleic acid sequence comprises a nucleotide sequence encoding a multidrug resistance gene. Alternatively, the method is carried out wherein the nucleic acid sequence comprises a nucleotide sequence encoding a mutated (mutant) protease and a nucleotide sequence encoding a mutated (mutant) reverse transcriptase, such as when the viral infection to be prophylactically or therapeutically treated is a retrovirus.

The method preferably further comprises administering to a host cell an agent selected from the group consisting of a cytotoxic drug, a protease inhibitor, and a reverse transcriptase inhibitor (i.e., in addition to administration of the vector).

Accordingly, a vector can be employed in accordance with the above-described method not only to treat therapeutically a viral infection but to protect a potential host cell from viral infection, i.e., a method of prophylactically treating a viral infection or a “vaccination” against a virus of interest, such as a RNA virus, in particular a retrovirus, such as HIV. The method essentially inhibits the replication of a wild-type strain of virus before the host cell comes into contact with the wild-type strain of virus. In this regard, the vector can comprise or encode proteins that block superinfection with a wild-type virus. The method comprises contacting the host cell with a conditionally replicating vector, as described above, and a “helper-expression vector,” i.e., a viral genome that promotes the replication of the “vector” in an uninfected host. The conditionally replicating vector comprises a selective advantage for packaging and/or propagation. Furthermore, the vector, for example, can contain a sequence that enhances cell survival, promotes viral production, induces apoptosis, facilitates protein production and/or promotes immune function and/or targeting. The “helper-expression vector” construct is any expression vector that complements for the inability of the “vector” to replicate. Such helper-expression vectors are common and are easily constructed by those of ordinary skill in the art. The helper-expression vector can be either packaged into virions, like the vector, or expressed without a packaging requirement. Since the “vector” has a selective advantage for packaging and/or propagation, this system provides a safe means to achieve high replication of the virus without the possible pathogenic effects that a live attenuated virus could potentially cause. In addition, the vector can be admixed with nonspecific adjuvants to increase immunogenicity. Such adjuvants are known to those skilled in the art, and include, but are not limited to Freund's complete or incomplete adjuvant, emulsions comprised of bacterial and mycobacterial cell wall components, and the like.

The conditionally replicating viral vectors, or preferably lentiviral vectors, of the invention may also be employed for immunotherapy in treating viral infection or for the treatment of oncogenic disorders, for example. Dendritic cells or their precursors (e.g. CD34+ hematopoietic cells or blood monocytes, but not limited to these cell types) as well as other antigen presenting cells, may be transduced with a vector that expresses HIV proteins or a protein sequence that contains the major epitopes by which a host mounts an immune response against a foreign agent such as a virus. Such protein epitopes for HIV are described in the “HIV molecular immunology database”, 1998, National Institutes of Allergy and Infectious Diseases, Maryland & Los Alamos National Laboratory, New Mexico, which is hereby incorporated as if fully set forth. A preferred embodiment for an HIV epitope protein is an integrated or composite CTL sequence (or a fragment thereof) set forth in Example 14 below. Other epitopes that could be similarly expressed are derived from other viruses, bacteria, fungi, parasites, tumor cells and genetically modified cells. More than one epitope may be consolidated into one protein sequence so that non-immunogenic sites are excluded to create a vector with increased safety because the protein coding sequences are severely discontinuous. A preferred embodiment of the discontinuous sequence is a degenerated discontinuous sequence (with or without appropriate linker amino-acid sequences to stabilize protein structure, if necessary) coding for the immunologically relevant epitopes. The use of degenerate sequences reduces the risk of recombination. Another preferred embodiment is expression of the described protein sequence (or a fragment thereof) via a spliced message driven by a retroviral LTR, such as the HIV-LTR.

A concern, however, with the use of retroviral vectors in the treatment of human infection and disease is the possibility of generation of replication competent virus (RCV). Homologous recombination between the helper vector and the conditionally replicating vector is likely one of the principal routes for RCV generation. Thus, the present invention provides for modification of the helper vector construct to minimize or eliminate the possibility of homologous recombination. Any modification that serves to decrease the probability of dimerization, copackaging, and/or recombination of the helper vector and the conditionally replicated vector is contemplated by the present invention.

An embodiment to the present invention is to insert one or more anti-vector ribozyme or antisense sequence (optionally in the form of a cassette, defined as at least two such sequences linked in tandem with respect to ribozymes and defined as at least two sequences linked in tandem and targeted to discontinuous regions of the targeted vector nucleic acid with respect to antisense sequences) into the 3′ end of helper gene coding sequences but 5′ to the transcription termination site. While non-specific packaging of helper genomes into vector may occur, it is likely that such packaging is vector dependent. Therefore a number of strategies may be used to decrease the packaging of helper vector into vector particles, which would otherwise be a first step in the possible generation of a RCV.

A preferred helper construct includes an anti-vector ribozyme or antisense molecule in the 3′ end of the structural protein coding sequence or the envelope (homologous or heterologous) coding sequence. A more preferred helper construct includes an anti-vector ribozyme or antisense molecule into the 3′ end of the structural protein coding sequence and the envelope coding sequence. If the nucleic acid sequence is an antisense molecule, then it can act both intracellularly to destroy co-localized double stranded vector-helper molecules through cellular nucleases. If the helper is packaged with the vector RNA into viral particles, then these molecules are unable to undergo complete reverse transcription to generate an RCV.

Another preferred embodiment place sequences on the helper construct that promotes differential tracking or localization of the helper nucleic acid away from that of the vector. For example, but not limited thereto, is the inclusion of heterologous intron and poly A sequences into the helper constructs, such as those shown in FIG. 6. These sequence would facilitate differential tracking of the vector and helper nucleic acids to different cellular or subcellular locations. Preferably, the titer of vector produced is not affected by more than 100 fold, more preferably no more than 10 fold, and most preferable not affected by such a helper modification.

FIG. 7 shows that the presence of one ribozyme (pVirPac1.2Rz) or a ribozyme and an intron designed to affect cellular trafficking of helper RNA (pVirPac1.2RzIn) had no significant effect on vector titer. FIG. 12 shows that PCR analysis of titer samples for co-packaged helper constructs indicated reduced co-packaging in the presence of a ribozyme versus high copackaging in the absence of a ribozyme. The presence of two ribozymes completely prevented the co-packaging of helper vector into vector viral particles. Thus the invention includes an effective means of using a two plasmid packaging system to both produce high vector titers and improve the safety of such vectors by reducing or eliminating the co-packaging of helper vector.

The data suggest that helper packaging into virions is not random, but vector dependent. An alternative preferred embodiment to prevent co-packaging of helper nucleic acids with that of the vector is to degenerate the helper construct in regions that are important for the association of helper and vector sequences. This approach can be further modified by completely degenerating the helper sequence to prevent co-packaging of helper and vector sequences.

For example, the nucleotide sequence of the helper vector can be degenerated either in part or in whole. While such degeneration considerably lessens or eliminates the likelihood of homologous pairing and recombination of the helper vector and conditionally replicating vector, it does not affect the ability of the helper vector to encode the protein products necessary for viral replication and packaging. This degeneration can be directed at every possible gene and open reading frame (ORF) that is homologous between the helper and conditionally replicating vectors or more specifically targeted to or within particular genes or ORFs within the helper vector. It should be noted that complete degeneracy is not necessary to reduce the likelihood of homologous recombination since the lack of sequence homology greater than 18 nucleotides in length should be sufficient. Thus degeneracy to levels such that no more than 18, 17, 16, 15, 14, 13, 12, 11, 10, 9, 8, 7 or 6 nucleotides are identical between sequences will suppress or prevent recombination. The sequences can be degenerated using codons that have a preferential expression in human or a particular cell type, if desired.

As an example, the nucleotide sequence encoding gag, rev, and/or tat can be degenerated either in whole or in part. Other examples include degenerating the first 42 nucleotides of the gag sequence, the first 208 nucleotides of the rev sequence, the last 183 nucleotides of the tat sequence, and the last 545 nucleotides of the Pol sequence. Examples of helper vectors with such degeneracy are pVirPac1.2, pVirPac1.2Rz, pVirPac1.2Rz2, and pVirPac1.2RzIn or their derivatives. Of course, the nucleotide sequence encoding any other viral gene product can also be degenerated according to the invention thereby diminishing, minimizing or eliminating the likelihood of homologous recombination.

An alternative embodiment of the present invention is the incorporation of an element in the helper vector that targets and degrades the conditionally replicating vector in the event the two vectors are co-localized, co-packaged or otherwise paired together. A ribozyme is an example of such an element. Any ribozyme that selectively targets a conditionally replicating vector is contemplated by the present invention. Multiple ribozymes, such as a double or triple ribozyme cassette as described herein, may also be used. For example, the ribozyme can target the U5 region (e.g., the U5 region of HIV-1, HIV-2, or another retrovirus used in the conditionally replicating vector) of the conditionally replicating vector. Cleavage of the conditionally replicating vector prevents a recombination event with a conditionally replicating vector from generating a RCV, which may occur if the two vectors were co-packaged. Examples of helper vectors containing a ribozyme are pVIRPAC-1.1 Rz, and pVIRPAC-1.2Rz2 as shown in FIG. 6.

Yet another embodiment of the present invention is the substitution of a heterologous RRE, including any retroviral RRE but preferably another lentiviral RRE, in the helper vector, such as the substitution of HIV-2 RRE for HIV-1 RRE, a CTE or a PRE. This approach contributes to diminishing, minimizing or eliminating the possibility of homologous recombination based on the different RREs having different sequences. A further advantage of such a substitution according to the invention is a surprising and unexpected increase in the production of conditionally replicating vector of as much as approximately five-fold. Without being bound by theory, the presence of different RREs between the helper vector and the conditionally replicating vector may be bound by one or more different cellular factors that may be limited in quantity. Thus the use of different RREs may reduce competition between the two vectors for limited cellular factors. Examples of helper vectors containing an HIV-2 RRE element for packaging a HIV-1 based retroviral vector are pVIRPAC-1.2, and pVIRPAC-1.2Rz2 as shown in FIG. 6

Yet another embodiment is the incorporation of in the helper vector of a nucleotide sequence encoding a heterologous env protein, such as VSV-G from vesicular stomatitis virus, Rabies G protein, GaLV, Alphavirus E1/2 glycoprotein, or RD114, an env protein from feline endogenous virus. This permits the conditionally replicating vector to be packaged into particles with heterologous env proteins. Furthermore, a ligand may be optionally inserted into the envelope protein to promote stimulation of the target cell during transduction, if desired. Since modifying the envelope protein may disrupt its binding ability, a preferred embodiment is to express both the modified ligand containing chimeric envelope protein with the unmodified pseudotyped envelope protein during production. Thus, both types of envelope proteins would be on the surface of the cell, one to stimulate the target cell, the other to mediate binding and entry. A preferred pseudotyped Lentiviral vector that also expresses a chimeric receptor for cellular stimulation is the VSV-G envelope protein and a chimeric VSV-G envelope protein. A more preferred chimeric envelope protein is an VSV-G/RD114 chimeric envelope protein (see FIGS. 14A and 14B). Other preferred proteins (or fragments thereof) to create chimeras with VSV-G include notch, delta, FLT-3 ligand, TPO, Kit ligand, a ligand that binds CD3, a ligand that binds CD28, and a ligand that binds GM-CSF.

The sequence encoding the heterologous env protein may optionally be operatively linked with a second inducible promoter to ensure a more abundant supply of env protein for viral packaging. This approach may further increase viral production, especially under conditions where env protein production is the rate limiting factor. The heterologous env protein encoding sequence may either be contained on the same helper vector as the other complementary viral protein encoding sequences or on a separate vector. Optionally, it may also be integrated into the production host cell or cell line.

A further embodiment of the present invention is the selective positioning of splice donor and acceptor sites on the helper vector such that the selective splicing out of certain viral components serves to minimize or eliminate the possibility of homologous recombination with the conditionally replication. For example, the splice sites might be located to permit the deletion of the RRE and/or the packaging or dimerization signals as the result of splicing events. Therefore the context of whether nucleic acids are expresses can be controlled by the placement of splice sites in the vector. For example, splice sites could be place distally to a ribozyme or antisense sequence so that the ribozymes or antisense is incorporated in unspliced but not spliced RNAs, for example. Alternatively, the ribozymes or antisense could be placed downstream of the splice site if the goal is to express the ribozymes or antisense molecule from all mRNA molecules and not a particular subtype.

When a vector is employed in accordance with the above-described method as a prophylactic treatment of viral infection, the vector can encode an antigen of a protein that is not encoded by a wild-type virus, such as a mutant viral protein or a nonviral protein. Accordingly, the antigen encoded by the vector can be of bacterial origin or cancerous cell origin, for example. Furthermore, the conditionally replicating viral vector, or preferably lentiviral vector, also can encode a MHC gene for proper presentation of the antigen to the host's immune system. Thus, such vectors can be used to facilitate a persistent immunological response against a diverse array of potential pathogens and/or endogenous proteins (e.g., tumor-specific antigen) that are selectively expressed in abnormal cells.

The above example does not limit the use of the invention to the delivery of an antigen to dendritic cells for immunotherapy. To the contrary, the invention provides means and methods for any gene of interest to be delivered and expressed in any desired cell type. Moreover, the invention also permits multiple genes to be delivered and expressed via a single vector. Multiple genes may be expressed by using any appropriate strategies for gene expression. A non-limiting example is the use of a Internal Ribosome Entry Site (IRES) which can be placed distally to a first gene that needs to be expressed. A preferred example is to express a gene of interest to an IRES sequence that is distal to a variant MGMT gene. A more preferred vector embodiment expresses the gene of interest and the IRES linked gene of interest from an unmodified or modified HIV-LTR promoter off a spliced message. A further preferred vector expresses the two genes of interest from an unmodified or modified HIV-LTR promoter that, if modified, is modified in the sequences that bind transcription factors. Of course any unmodified or modified lentiviral LTRs may be used in addition to HIV LTRs.

Another non-limiting example is to express the gene of interest from the HIV-LTR using splicing sites that are derived from HIV. For example one gene of interest could be expressed from the Nef splice acceptor site while a second gene of interest could be expressed from the Tat splice acceptor site. In this way two genes could be expressed without the need for an IRES element. Any splice site could be used including those of the Tat, Rev, Vif, Vpr, Vpu, Nef and Env genes. The Env protein is expressed from a singly spliced message so expressing a gene of interest via this splice site may require Rev, if significant expression of the gene of interest is required. Furthermore, expression from the HIV-LTR may be made Tat and Rev dependent, if desired.

Furthermore, the “helper-virus” (also referred to herein as “helper”) expression vector can be engineered to express only in specific cell types (e.g., stem cells, professional antigen presenting cells, and tumor cells) by the addition or omission of a specific genetic element/factor (either in the vector or helper-virus expression construct), which permits cell-specific vector replication and spread. Thus, the vector still spreads by complementation with the helper-virus construct, but this spread is cell-specific, depending upon whether a certain genetic element/factor is added to or omitted from the vector or helper-virus expression construct. This can be used alone or in combination with other of the above-mentioned strategies.

For example, a conditionally replicating HIV vector can be designed to replicate specifically in macrophages, rather than in T-cells. The vector, which would constitute a Tat-defective HIV (the vector encodes the other HIV proteins but they are not expressed because of the absence of the Tat transcriptional transactivator), can encode a ribozyme that cleaves wildtype HIV but not conditionally replicating HIV RNA. The helper-expression vector for this vector can encode a tat gene expressed off of a macrophage-specific promoter. Thus, the crHIV would conditionally replicate only in macrophage cells, while not being able to replicate in T-cells or other cell types.

Alternatively, the tat gene can be operably linked to a tumor-specific promoter; thus, the crHIV would then replicate only in tumor CD4 cells and not in normal CD4 cells. The genetic element/factor also can be a modification of a promoter sequence of the vector such that it is expressed only in specific cell types and not in other cell types in concert with the “helper-virus” expression construct.

In another embodiment, the helper-expression construct or the vector construct envelope proteins (if such constructs are engineered to contain envelope proteins) can be modified so that the vector-virion will specifically infect certain cell types (e.g., tumor cells), while not being able to infect other cell types (e.g., normal cells). In yet another embodiment, an adenovirus, which is lacking one or several key factors for replication, could be complemented by using a helper construct, which provides such factors linked to a tumor-specific promoter. Thus, the factors that complement replication of the adenovirus would only be expressed in tumor cells, thereby permitting viral replication in tumor cells (with expression of proteins required for cell killing), but not in normal cells.

In a further embodiment, a vector could express a negative selection gene for the killing of cells by using an O⁶-guanine alkylating agent such as, but not limited to, BCNU (or a functional analog of BCNU) as a negative selection drug. A lentiviral vector expressing an antisense or a ribozyme targeted to MGMT gene can be delivered to normal cells. At a later desirable timepoint, the cells can be treated with BCNU, or its analog, either ex vivo or in vivo, to kill the vector transduced cells. In a preferred embodiment normal cells are transduced with the MGMT antisense expressing vector prophylactically and are later killed when the cells have become abnormal. An example of this latter approach is where the transduced cells become cancer cells which could then be killed by treatment of the cells with BCNU, or its functional analog. Another preferred embodiment is to express an anti-MGMT antisense or ribozymes molecule from the U1 promoter and contained within the U1 snRNA. An even more preferred embodiment is to transduce the anti-MGMT lentiviral vector into a population of hematopoietic cells such as lymphocytes, stem cells and dendritic cells.

In another preferred embodiment, the normal cells are hematopoietic cells, preferably T cells, that are transduced with the vector before transplantation into an allogeneic host. Such cells may be killed by treatment with BCNU, or its functional analog, if the cells become detrimental to the host (e.g. if graft versus host reactions occurs). In yet another embodiment, the same anti-MGMT antisense or ribozyme expressing lentiviral vector could be used for purging undesirable cells from mixed cell populations. A non-limiting example is in the case of cancer cell contaminated bone marrow that is ready for transplantation. Tumor cells have been observed to be very efficiently transduced at relative low MOIs in a single round of transduction (See FIG. 13A). In contrast, normal cells, especially but not limited to CD34+ hematopoietic stem cells, are more difficult to transduce efficiently, requiring multiple rounds of transduction for high efficiency (See FIG. 13B). Therefore an attractive purging strategy is via transduction of contaminated bone marrow with a anti-MGMT antisense containing vector using an MOI that would efficiently transduce the contaminating tumor cells but not the normal cells. This example does not limit the scope of the invention to either ex vivo uses or solely in cancer related applications. Other applications include in vivo selective gene delivery, especially but not limited to the treatment of brain cancers, and any other disease where there is a differential efficiency of transduction between diseased and normal cells which can be exploited for targeting by the present invention.

Thus, the present invention also provides a method of treating cancer, and in particular, treating T-cell leukemia. “Treating cancer” according to the invention comprises administering to a host a further modified vector as set forth herein for the purpose of effecting a therapeutic response. Such a response can be assessed, for example, by monitoring the attenuation of tumor growth and/or tumor regression. “Tumor growth” includes an increase in tumor size and/or the number of tumors. “Tumor regression” includes a reduction in tumor mass.

“Cancer” according to the invention includes cancers that are characterized by abnormal cellular proliferation and the absence of contact inhibition, which can be evidenced by tumor formation. The term encompasses cancer localized in tumors, as well as cancer not localized in tumors, such as, for instance, those cancer cells that expand from a tumor locally by invasion, or systemically by metastasis. Theoretically, any type of cancer can be targeted for treatment according to the invention. Preferably, however, the cancer is of viral origin.

Finally, the above-described vectors can be directly used for in vivo gene therapy. Current strategies for gene therapy suffer because they cannot mediate gene delivery to large percentage of cells; only a certain percentage of the cells are infected. This is especially important in anti-tumor strategies where gene transduction of the entire tumor population is crucial. By adding the “vector” in concert with a “helper,” the immediately transduced cells will produce viral particles that can infect neighboring cells and thus enable high and possible complete transduction efficiency. In one embodiment to this invention, a human retrovirus (which could be HIV or a retrotransposon element) could be delivered into tissue (or cells in vitro) with a “helper” construct. Cells immediately containing the vector and helper will produce virus and will package the vector conditionally into virions. These virions will be able to mediate high efficiency transduction of neighboring cells (since cell-cell contact is the most efficient means to transduce cells) The immediately transduced cells may or may not die, depending whether the vector/helper combination results in a cytolytic infection. In the case of a retrotransposon, the helper may not need to contain structural proteins since normal or tumor cells may contain the protein/factor necessary for encapsidation into virions. In this case the helper can merely be, but not restricted to, a transactivator protein that activates transcription of the factors required for retrotransposon encapsidation. In the case of HIV, other factors may, but not necessarily, be required for encapsidation of the HIV genome into progeny virions for infection/transduction of cells.

The above-described vectors also can be used in counter-biological and counter-chemical warfare strategies. For example, a conditionally replicating vector can be delivered into an individual recently infected with a highly pathogenic virus or bacterium or a chemical agent (e.g., toxin). The vector would interfere with the replication of the pathogenic virus as described previously. However, the conditionally replicating vector also can be used for antibacterial or anti-chemical strategies in concert with a helper-expression vector (“helper”).

For example, a conditionally replicating vector can secrete anti-bacterial or anti-toxin antibodies after a “helper” permits its expression and propagation. The “helper” can be, but not necessarily, driven off an inducible promoter that permits its expression upon activation by a bacterium, a cytokine (in response to bacterial infection), an antibiotic (as with the tetracycline inducible promoter systems (Paulus et al., J. Virol., 70, 62-67 (1996)) or a chemical (e.g., the toxin, itself). Thus, the conditionally replicating vector would not only selectively propagate with the aid of the “helper” in response to the incurring pathogen or toxin (as a result of activation of the helper) but also secrete anti-pathogen or anti-toxin antibodies to inhibit the pathological effects of the tumor antigen, pathogen or chemical (e.g., toxin). Thus, any protein, factor, or genetic element that can be transcribed into either mRNA and/or protein can be inserted into a conditionally replicating vector to inhibit a pathogenic response—in concert with a “helper,” which promotes its selective propagation and expression (selective because the products of the helper are expressed conditionally (for example, but not restricted to, (a) an inducible promoter system—a factor in a tumor cell activates the production of a helper factor, a toxin responsive sequence that expresses a helper factor, or a cytokine responsive promoter induces production of a helper factor, (b) a helper RNA/protein/factor is selectively stabilized in certain cells and not in others), and (c) indirect induction of a third party gene that affects helper viral protein production, chaperoning, targeting, structure or another biofunction). Such strategies can be used in transgenic plants and animals to protect them from pathogens. Similarly, such strategies can be used in transgenic systems to produce heterologous proteins/factors of value.

In another embodiment of a method in accordance with the present invention, a cell line can be developed for screening a drug/factor to determine, for example, which part of the protein/factor is important for a particular function. A vector can be created to express a mutagenized protein of interest within a given cell line. The RNA encoding the mutagenized protein, however, is made resistant to the ribozyme by insertion of silent point mutations, for example. Wild-type protein expression, however, is inhibited within the cell line. Vectors that express a ribozyme to the protein of interest also can be constructed to express mutant test protein. When the vector is transduced into the cells, most of the native RNA encoding the normal protein is cleaved, whereas the mutant test protein is expressed. This method can be used with recently developed delivery and selection techniques as a quick and powerful technique to determine how a given protein functions and how a given factor/drug interacts with the protein.

In yet another embodiment for the application of vector to treat blood-borne diseases, the patient undergoes leukapharesis so as to extract sufficient quantities of white cells from the blood. After leukapharesis the desired cells are isolated, transduced with the vector and then expanded in culture to the desired cell number. During ex vivo expansion of the desired cell type, antibodies targeted to a cell surface protein on the desired cell type are infused in the patient, with the goal of destroying these cells during a period of time that is coincident with expansion of sufficient quantities of the desired cell type. The transduced cells are then reinfused back into the patient to compensate for the in vivo antibody mediated loss of cells.

A preferred means of practicing the above is with the isolation of CD4+ T cells from the leukapharesed material and then transducing the cells with a HIV vector that contains an anti-HIV antisense or ribozyme sequence for expansion ex vivo. During ex vivo expansion of the cells, the patient's endogenous CD4+ T cells are destroyed by the administration of, for example, Anti-thymocyte Globulin (ATG) (Pasteur Merieux Serums et Vaccins, Lyon, France and distributed by SangStat Medical Corporation, Menlo Park, Calif., USA) or Atgam (Upjohn Company, Kalamazoo, Mich., USA), but any cytoreducive antibody could be used after appropriate screening that would be obvious to an ordinary practitioner of the art. After expansion the transduced CD4+ T cells are then infused back into the patient. In a preferred embodiment, the CD4 negative cells obtained during the isolation of the CD4 positive cells are retained, frozen and thawed for infusion at the time the transduced CD4 positive cells are infused back into the patient.

The above example does not limit the scope of the invention to either ATG nor CD4+ T cells. Any cell type may be targeted by use of an antibody that binds a cell surface protein on said cell and is cytocidal. The antibody could be introduced exogenously into the patient or a second vector secreting the antibody could be introduced into the body. This vector could produce the antibody either constitutively (for the life of the cell), transiently (for example, but not limited to an integrase minus vector) or inducibly (for example, but not limited to a tetracycline inducible promoter system).

In yet another embodiment of the invention, any vector of the invention may be used to prevent or inhibit a productive viral replication by interfering with the production of viral DNA. Without being bound by theory, such interference may be by disruption of DNA production or replication, such as that of first or second DNA strand synthesis in the reverse transcription process or DNA polymerase medicated replication, inhibiting the integration of viral DNA into the genome of the host cell, or increasing the degradation or instability of the viral DNA.

Viral DNA is any DNA that is produced as part of a viral life cycle. Non-limiting examples include retroviruses, including lentiviruses, adenoviruses and adeno-associated viruses, herpes viruses and hepadna viruses. For viruses with a life cycle that includes integration into the host cell genome, viral DNA also refers to the form of the viral genome that is to be integrated or already integrated into the host cell's genome.

For direct interference with viral integration, the invention includes the use of the respective viral vector(s) that have an integrative step in their replication cycle. Preferably, the integration of vector occurs before viral infection occurs. The integration of vector is preferably effected with a multiplicity of infection (“MOI”) greater than one, more preferably from about 2 to about 10, about 20, or about 50. MOI of up to about 100 or 200 may also be used with highly concentrated vector preparations and depending on cell type. Particularly preferred are MOI values from about 2 to about 20, about 2 to about 15, about 2 to about 10, or about 2 to about 5. These values are appropriate for use with primary cells. The therapeutic applications of such values may be practiced by treating host cells in vitro or ex vivo followed by introducing or returning them to a patient or subject.

The vector may also be constructed to contain an anti-viral genetic payload or could be devoid of such a payload in instances where it is not required. Furthermore, vectors may be used to inhibit production of the viral DNA of a wild type virus that is different from the virus that the viral vector is derived from. For example, a vector derived from the human immunodeficiency virus could inhibit the production, or integration, of hepatits A or B viral DNA in hepatocytes.

Without being bound by any theory regarding the mechanism by which inhibition of viral DNA production occurs, and as shown in FIG. 19 and Example 12 below, that the vector prevents productive wild type replication by inhibiting wildtype HIV-1 (wt-HIV) DNA formation by the incoming infectious wt-HIV, denying the wild type virus an important step in its life cycle. FIG. 19 shows the effects of a HIV based vector (cPT2 as described above) upon DNA formation of wt-HIV introduced into primary human CD4 T lymphocytes. Because the method of detecting wt-HIV used in the figure also detects integrated forms of the DNA, the vector may interfere with wt-HIV DNA formation by preventing its integration (such as by saturating HIV integration sites) and thus allowing it to be degraded or otherwise lost from the cell.

The invention is also not limited to the use of viral vectors but may instead be combined with other anti-virals, such a drug regimen, that inhibits productive viral infections at any point in the viral life cycle, including wild type virus integration or productive replication. Prevention of wild type virus integration by alternative and complementary (e.g. drug+vector combination) modalities allows a combined vector and drug dose that requires less vector but still inhibits productive viral replication effectively in vivo.

There also are numerous uses of the method and the vectors of the present invention in vitro. For instance, the vectors can be employed to ascertain certain nuances of viral replication and ribozyme function. Similarly, the ribozyme-containing vectors can be used as diagnostic tools, e.g., to assess mutations present in diseased cells, or to examine genetic drift. The vectors may also be used to determine or confirm the function of a gene, whether the function is previously known, unknown, or merely suggested or contemplated. This aforementioned discussion is by no means comprehensive regarding the use of the present invention.

Benefits of the Invention

The advantages of using a crHIV strategy for genetic therapeutic treatment of AIDS and other diseases are considerable. For instance, the problem of targeting the vector to cells infected by HIV becomes resolved. After in vivo transfection of crHIVs into infected CD4+ cells, the crHIVs become packaged into progeny virions using the endogenous infectious HIV envelope proteins. Thus, the crHIV RNA tags along inside progeny virions and infects cell types that are normally infectable by that particular strain of HIV, producing nonpathogenic virions. This includes difficult to target cells, such as the microglia of the brain, which are a major reservoir for HIV infection of the central nervous system. There is likely to be little toxicity associated with crHIV vectors that infect uninfected CD4+ cells, since no viral proteins are coded by crHIV vectors. Moreover, the result of crHIV vector competition with wild-type HIV results in the production of nonpathogenic particles, which results in decreased viral loads. Decreasing pathogenic HIV-1 loads can not only increase the survival time of infected individuals, but also can decrease the rate of transmission to uninfected individuals, since the crHIV particles also can spread systemically (i.e., as does infectious HIV). Decreased pathogenic HIV-1 loads in the blood can be particularly important in pregnant HIV-infected individuals, since the production of crHIVs can also decrease transmission of HIV-1 from infected mothers to their fetuses in utero.

The plasmid DNA/lipid mixture that can be employed for introducing the crHIV vector into host cells should be stable and cheap to produce, bypassing expensive ex vivo strategies. Of course, the method of the invention is inherently flexible inasmuch as it could also be employed for ex vivo gene delivery, should this be desired. Regardless, the availability of the liposome-mediated approach opens the possibility for treatment of the general population—something that is not feasible with current gene therapeutic strategies. The crHIV vectors also can be engineered to contain several ribozymes, which can be made to different targets on the HIV genome. This reduces the possibility that infectious HIV can mutate and escape the effect of the anti-HIV ribozymes. Furthermore, the conditionally replication competent virus strategy can be applied to treat other viral infections, especially those where viral turnover is high.

A particularly useful feature of crHIV vectors is that they can be employed to express genetic antiviral agents, for instance, a ribozyme, post-transcriptionally. Thus, infection of uninfected cells with crHIV vectors results in low toxicity because little expression occurs from the HIV long-terminal repeat (LTR) promoter in the absence of the Tat protein. High levels of crHIV expression, and its consequent antiviral activity, occurs only when the Tat protein is provided by complementation with wild-type HIV. Thus, crHIV vectors are not designed to protect cells from HIV infection, but to lower the overall wild-type HIV viral burden through selective accumulation of nonpathogenic crHIV particles.

While not seeking to be bound by any particular theory regarding the operation or functioning of the invention, it is believed that ribozymes can be employed as confirmed in the following Examples to provide crHIV genomes with a selective advantage because of two useful properties: (1) they have a high degree of specificity, and (2) they have a relative efficiency, depending upon their ability to co-localize with target RNAs (Cech, Science, 236, 1532-1539 (1987)). The specificity of ribozymes is conferred by their specific hybridization to complementary target sequences containing a XUY site. Ribozymes are relatively efficient because they cleave target RNAs with high efficacy only when they efficiently co-localize with target RNAs. In a mixed HIV/crHIV infection, co-localization of ribozyme-containing crHIV RNAs with wild-type HIV RNAs must occur, since HIV RNA genomes dimerize prior to packaging into progeny virions. Cleavage of non-genomic species of wild-type HIV RNAs, required for the production of viral proteins, is likely to be less efficient than that of genomic wild-type HIV RNAs inasmuch as non-genomic HIV RNAs no not dimerize. It was discovered in the experiments described herein that the selective advantage conferred to crHIV RNAs was due to the selective packaging of crHIVs into viral particles. These results suggest that most efficient cleavage occurs intracellularly during dimerization, resulting in the selective destruction of wild-type HIV RNAs by host nucleases. This allows for the preferential packaging of crHIV RNAs into viral particles.

The application of crHIV vectors for HIV therapy can involve not only genomic selection of crHIVs, but also cellular selection of cells producing crHIV particles. Otherwise, the cells producing wild-type HIVs will produce wild-type HIV particles at a selective advantage over the cells producing crHIV particles, and will rapidly predominate. A selective advantage can be conferred to crHIV expressing cells by inserting a gene into crHIV genomes (e.g., the multidrug resistance gene) that confer crHIV expressing cells (in the presence of drug) with a survival advantage over cells expressing wild-type HIV. Under these conditions, wild-type HIV-expressing cells progressively die, but still produce some wild-type HIV, while crHIV-expressing cells that selectively produce crHIV survive. Infection of crHIV-containing cells with remaining wild-type HIV will result in the further production of crHIV containing viral particles. Thus, a viral genomic shift can result with the cumulative infection of CD4+ cells with crHIV genomes, thereby altering the viral balance in the host from pathogenic wild-type HIV to nonpathogenic crHIV genomes. Such a strategy can result in clearance of wild-type HIV, once the balance of HIV genomes selectively shifts from wild-type HIV to crHIV. Viral replication eventually ceases, since crHIVs can only replicate in the presence of wild-type HIV helper genomes. Therefore, under such mutually restrictive conditions, it can be possible to engineer crHIV vectors that not only decrease wild-type HIV viral loads, but also clear the virus from the HIV-infected host.

Means of Production The vector can be produced by the process of complementation either by using transient transfection of vector and helper genomes into a cell line, preferably, but not limited to, the well known 293T cell line; or by using a packaging cell line. Preferably the cell is transiently transfected at a high transfection efficiency using a transfection reagent, preferably calcium phosphate, electroporation or a lipid transfection reagent. Once the transfection has occurred, the vector is harvested from the supernatant preferably not less than 12 hours after transfection and not more than 7 days after transfection. The vector can be concentrated, optionally if desired, by high speed centrifugation without precipitation or ultracentrifugation. Preferred centrifugation conditions are at about 5000-12,000×g, more preferably at about 10,000×g. More preferred conditions are centrifugation overnight at 4° C.

Methods for Vector Purification

Method 1

1. Clarification of viral supernatant

2. Concentration by ultrafiltration

3. Diafiltration with or without benzonaze treatment

4. Ion-exchange chromatography

5. Size exclusion chromatography or diafiltration

6. Optionally concentration by ultrafiltration

Method 2

1. Clarification of viral supernatant

2. Ion exchange chromatography

3. Diafiltration or size exclusion chromatography

4. Optional benzonaze treatment

5. Size exclusion chromatography or diafiltration

6. Optionally concentration by ultrafiltration

Different resins can be used for the above procedure, including Poros 50HQ from Perseptive Biosystems. Hollow fiber cartridges can be used for ultrafiltration or diafiltration, including cartridges (UFP-750 series) from A/G Technologies

Means of Administration

According to the invention, a vector is introduced into a host cell in need of gene therapy for viral infection as previously described. The means of introduction comprises contacting a host capable of being infected with a virus with a vector according to the invention. Preferably, such contacting comprises any means by which the vector is introduced into a host cell; the method is not dependent on any particular means of introduction and is not to be so construed. Means of introduction are well-known to those skilled in the art, and also are exemplified herein.

Accordingly, introduction can be effected, for instance, either in vitro (e.g., in an ex vivo type method of gene therapy) or in vivo, which includes the use of electroporation, transformation, transduction, conjugation or triparental mating, transfection, infection, membrane fusion with cationic lipids, high-velocity bombardment with DNA-coated microprojectiles, incubation with calcium phosphate-DNA precipitate, direct microinjection into single cells, and the like. Other methods also are available and are known to those skilled in the art.

Preferably, however, the vectors or ribozymes are introduced by means of cationic lipids, e.g., liposomes. Such liposomes are commercially available (e.g., Lipofectin™., Lipofectamine™, and the like, supplied by Life Technologies, Gibco BRL, Gaithersburg, Md.). Moreover, liposomes having increased transfer capacity and/or reduced toxicity in vivo (e.g., as reviewed in PCT patent application no. WO 95/21259) can be employed in the present invention. For liposome administration, the recommendations identified in the PCT patent application no. WO 93/23569 can be followed. Generally, with such administration the formulation is taken up by the majority of lymphocytes within 8 hr at 37° C., with more than 50% of the injected dose being detected in the spleen an hour after intravenous administration. Similarly, other delivery vehicles include hydrogels and controlled-release polymers.

The form of the vector introduced into a host cell can vary, depending in part on whether the vector is being introduced in vitro or in vivo. For instance, the nucleic acid can be closed circular, nicked, or linearized, depending on whether the vector is to be maintained extragenomically (i.e., as an autonomously replicating vector), integrated as a provirus or prophage, transiently transfected, transiently infected as with use of a replication-deficient or conditionally replicating virus, or stably introduced into the host genome through double or single crossover recombination events.

Prior to introduction into a host, a vector of the present invention can be formulated into various compositions for use in therapeutic and prophylactic treatment methods. In particular, the vector can be made into a pharmaceutical composition by combination with appropriate pharmaceutically acceptable carriers or diluents, and can be formulated to be appropriate for either human or veterinary applications.

Thus, a composition for use in the method of the present invention can comprise one or more of the aforementioned vectors, preferably in combination with a pharmaceutically acceptable carrier. Pharmaceutically acceptable carriers are well-known to those skilled in the art, as are suitable methods of administration. The choice of carrier will be determined, in part, by the particular vector, as well as by the particular method used to administer the composition. One skilled in the art will also appreciate that various routes of administering a composition are available, and, although more than one route can be used for administration, a particular route can provide a more immediate and more effective reaction than another route. Accordingly, there are a wide variety of suitable formulations of the composition of the present invention.

A composition comprised of a vector of the present invention, alone or in combination with other antiviral compounds, can be made into a formulation suitable for parenteral administration, preferably intraperitoneal administration. Such a formulation can include aqueous and nonaqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. The formulations can be presented in unit dose or multidose sealed containers, such as ampules and vials, and can be stored in a freeze-dried (lyophilized) condition requiring only the addition of the sterile liquid carrier, for example, water, for injections, immediately prior to use. Extemporaneously injectable solutions and suspensions can be prepared from sterile powders, granules, and tablets, as described herein.

The vector can be stored in any suitable solution, buffer or lyophilizable form, if desired. A preferred storage buffer is Dulbecco's Phosphate Buffered Saline; Dulbecco's Phosphate Buffered Saline mixed with a 1-50% solution of trehalose in water (1:1), preferably a 10% solution of trehalose in water (1:1), such that the final concentration is 5% trehalose; Dulbecco's Phosphate Buffered Saline mixed with a 1-50% solution of glucose in water (1:1), preferably a 10% solution of glucose in water (1:1), such that the final glucose concentration is 5%; 20 mM HEPES-buffered saline mixed with 1-50% solution of trehalose in water (1:1), preferably a 10% solution of trehalose in water (1:1), such that the final trehalose concentration is 5%; or; Dulbecco's Phosphate Buffered Saline mixed with a 1-50% solution of mannitol in water (1:1), preferably a 5% solution of mannitol in water (1:1), such that the final mannitol concentration is 2.5%.

A formulation suitable for oral administration can consist of liquid solutions, such as an effective amount of the compound dissolved in diluents, such as water, saline, or fruit juice; capsules, sachets or tablets, each containing a predetermined amount of the active ingredient, as solid or granules; solutions or suspensions in an aqueous liquid; and oil-in-water emulsions or water-in-oil emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers.

An aerosol formulation suitable for administration via inhalation also can be made. The aerosol formulation can be placed into a pressurized acceptable propellant, such as dichlorodifluoromethane, propane, nitrogen, and the like.

Similarly, a formulation suitable for oral administration can include lozenge forms, that can comprise the active ingredient in a flavor, usually sucrose and acacia or tragacanth; pastilles comprising the active ingredient in an inert base, such as gelatin and glycerin, or sucrose and acacia; and mouthwashes comprising the active ingredient in a suitable liquid carrier; as well as creams, emulsions, gels, and the like containing, in addition to the active ingredient, such carriers as are known in the art.

A formulation suitable for topical application can be in the form of creams, ointments, or lotions.

A formulation for rectal administration can be presented as a suppository with a suitable base comprising, for example, cocoa butter or a salicylate. A formulation suitable for vaginal administration can be presented as a pessary, tampon, cream, gel, paste, foam, or spray formula containing, in addition to the active ingredient, such carriers as are known in the art to be appropriate. Similarly, the active ingredient can be combined with a lubricant as a coating on a condom.

The dose administered to an animal, particularly a human, in the context of the present invention should be sufficient to effect a therapeutic response in the infected individual over a reasonable time frame. The dose will be determined by the potency of the particular vector employed for treatment, the severity of the disease state, as well as the body weight and age of the infected individual. The size of the dose also will be determined by the existence of any adverse side effects that can accompany the use of the particular vector employed. It is always desirable, whenever possible, to keep adverse side effects to a minimum.

The dosage can be in unit dosage form, such as a tablet or capsule. The term “unit dosage form” as used herein refers to physically discrete units suitable as unitary dosages for human and animal subjects, each unit containing a predetermined quantity of a vector, alone or in combination with other antiviral agents, calculated in an amount sufficient to produce the desired effect in association with a pharmaceutically acceptable diluent, carrier, or vehicle. The specifications for the unit dosage forms of the present invention depend on the particular compound or compounds employed and the effect to be achieved, as well as the pharmacodynamics associated with each compound in the host. The dose administered should be an “antiviral effective amount” or an amount necessary to achieve an “effective level” in the individual patient.

Since the “effective level” is used as the preferred endpoint for dosing, the actual dose and schedule can vary, depending on interindividual differences in pharmacokinetic's, drug distribution, and metabolism. The “effective level” can be defined, for example, as the blood or tissue level desired in the patient that corresponds to a concentration of one or more vector(s) according to the invention, which inhibits a virus, such as HIV, in an assay predictive for clinical antiviral activity of chemical compounds. The “effective level” for compounds of the present invention also can vary when the compositions of the present invention are used in combination with zidovudine or other known antiviral compounds or combinations thereof.

One skilled in the art can easily determine the appropriate dose, schedule, and method of administration for the exact formulation of the composition being used, in order to achieve the desired “effective level” in the individual patient. One skilled in the art also can readily determine and use an appropriate indicator of the “effective level” of the compounds of the present invention by a direct (e.g., analytical chemical analysis) or indirect (e.g., with surrogate indicators of viral infection, such as p24 or reverse transcriptase for treatment of AIDS or AIDS-like disease) analysis of appropriate patient samples (e.g., blood and/or tissues).

Further, with respect to determining the effective level in a patient for treatment of AIDS or AIDS-like disease, in particular, suitable animal models are available and have been widely implemented for evaluating the in vivo efficacy against HIV of various gene therapy protocols (Sarver et al. (1993b), supra). These models include mice, monkeys and cats. Even though these animals are not naturally susceptible to HIV disease, chimeric mice models (e.g., SCID, bg/nu/xid, NOD/SCID, SCID-hu, immunocompetent SCID-hu, bone marrow-ablated BALB/c) reconstituted with human peripheral blood mononuclear cells (PBMCs), lymph nodes, fetal liver/thymus or other tissues can be infected with vector or HIV, and employed as models for HIV pathogenesis and gene therapy. Similarly, the simian immune deficiency virus (SIV)/monkey model can be employed, as can the feline immune deficiency virus (FIV)/cat model.

These models can also be used to determine the safety of a vector for the purposes of validation of the vector system for clinical trials. An important application is the use of these animal models for biodistribution studies. Transduced cells, preferably but not limited to human cells, containing vector are injected into a non-human animal model and the safety of the vector is determined by the absence of vector genetic material in animal tissue. The absence of vector genetic material in animal tissue would mean that the vector does not autonomously replicate without the presence of the helper or wild-type virus and thus would be considered safe for clinical use in humans. The presence of the vector in the absence of a helper vector or helper virus could be considered a safety risk if the vector is not expected to replicate autonomously. However, in the instance that the vectors are expected to autonomously replicate, then other criteria for safety need to be established, for example the lack of replication in certain tissues or the level of replication in the animal. The presence or absence of the vector could be determined by PCR, or by FACS analysis if the tested vector expresses a marker gene that can be visualized by FACS, but is not limited to such means of detection.

Generally, an amount of vector sufficient to achieve a tissue concentration of the administered ribozyme (or vector) of from about 5 μg/kg to about 300 mg/kg of body weight per day is preferred, especially of from about 10 μg/kg to about 200 mg/kg of body weight per day. In certain applications, e.g., topical, ocular or vaginal applications, multiple daily doses are preferred. Moreover, the number of doses will vary depending on the means of delivery and the particular vector administered.

In the treatment of some virally infected individuals, it can be desirable to utilize a “mega-dosing” regimen, wherein a large dose of a vector is administered, time is allowed for the compound to act, and then a suitable reagent is administered to the individual to inactivate the active compound(s). In the method of the present invention, the treatment (i.e., the replication of the vector in competition with the virus being treated) is necessarily limited. In other words, as the level, for instance, of HIV decreases, the level of vector dependent on HIV for production of virions will also decrease.

The pharmaceutical composition can contain other pharmaceuticals, in conjunction with a vector according to the invention, when used to therapeutically treat AIDS. These other pharmaceuticals can be used in their traditional fashion (i.e., as agents to treat HIV infection), as well as more particularly, in the method of selecting for crHIV viruses in vivo. Such selection as described herein will promote conditionally replicating HIV spread, and allow conditionally replicating HIV to more effectively compete with wild-type HIV, which will necessarily limit wild-type HIV pathogenicity. In particular, it is contemplated that an antiretroviral agent be employed, such as, preferably, zidovudine. Further representative examples of these additional pharmaceuticals that can be used in addition to those previously described, include antiviral compounds, immunomodulators, immunostimulants, antibiotics, and other agents and treatment regimes (including those recognized as alternative medicine) that can be employed to treat AIDS. Antiviral compounds include, but are not limited to, ddI, ddC, gancylclovir, fluorinated dideoxynucleotides, normucleoside analog compounds such as nevirapine (Shih et al., PNAS, 88, 9878-9882 (1991)), TIBO derivatives such as R82913 (White et al., Antiviral Research, 16, 257266 (1991)), and BI-RJ-70 (Shih et al., Am. J. Med., 90(Suppl. 4A), 8S-17S (1991)). Immunomodulators and immunostimulants include, but are not limited to, various interleukins, CD4, cytokines, antibody preparations, blood transfusions, and cell transfusions. Antibiotics include, but are not limited to, antifungal agents, antibacterial agents, and anti-Pneumocystis carinii agents.

Administration of the virus-inhibiting compound with other anti-retroviral agents and particularly with known RT inhibitors, such as ddC, zidovudine, ddI, ddA, or other inhibitors that act against other HIV proteins, such as anti-TAT agents, will generally inhibit most or all replicative stages of the viral life cycle. The dosages of ddC and zidovudine used in AIDS or ARC patients have been published. A virustatic range of ddC is generally between 0.05 μM to 1.0 μM. A range of about 0.005-0.25 mg/kg body weight is virustatic in most patients. The dose ranges for oral administration are somewhat broader, for example 0.001 to 0.25 mg/kg given in one or more doses at intervals of 2, 4, 6, 8, and 12; etc., hr. Preferably, 0.01 mg/kg body weight ddC is given every 8 hr. When given in combined therapy, the other antiviral compound, for example, can be given at the same time as a vector according to the invention, or the dosing can be staggered as desired. The vector also can be combined in a composition. Doses of each can be less, when used in combination, than when either is used alone.

### EXAMPLES

The present inventive compounds and methods are further described in the context of the following examples. These examples serve to illustrate further the present invention and are not intended to limit the scope of the invention.

### Example 1

This example describes the construction of conditionally replication competent vectors according to the invention. In particular, this example describes the construction of conditionally replicating vectors based on HIV, i.e., crHIV vectors.

One of the most prominent aspects of HIV-1 pathogenesis is the production of genetic variants of the virus. The rapid production of HIV variants in vivo indicates that the virus can be considered within the framework of Darwinian genetic modeling (see, e.g., Coffin, Curr. Top. Microbiol. Immunol., 176, 143-164 (1992); and Coffin, Science, 267, 483-489 (1995)). The variants are a result of the infidelity of the HIV-1 reverse transcriptase molecule, which creates mutations in newly transcribed proviruses from viral genomic RNA. Therefore, under in vivo conditions of no significant bottlenecks and many replicative cycles, a substantial degree of genetic variation occurs with the production of many viral variants. Yet, wild-type HIV still predominates, since, under such unrestricted conditions, it has the highest selective advantage. However, in the presence of an inhibitor, for instance zidovudine, a viral variant will be selected that is conferred with a higher selective advantage than the wild-type strain, and consequently will predominate (Coffin (1992) and (1995), supra). Based on this, the present invention provides a conditionally replicating viral vector strategy that affords nonpathogenic HIV-1 genomes with a selective advantage over pathogenic wild-type HIV-1.

These nonpathogenic, conditionally replicating HIV (crHIV) vectors are defective HIVs that undergo replication and packaging only in cells that are infected with wild-type HIV. crHIV genomes compete with and decrease pathogenic wild-type HIV viral loads. The effect of decreasing wild-type HIV viral loads in an infected host should lead to an increased life expectancy. It should also decrease the ability of infected hosts to transmit wild-type HIV to uninfected individuals. For successful competition of crHIVs with wild-type HIV-1, two factors appear important: (1) a selective advantage of crHIV genomes over wild-type HIV genomes, and (2) a selective advantage of crHIV-expressing cells over cells expressing wild-type HIV (i.e., a selective advantage for the production of crHIV virions from crHIV-expressing cells over cells expressing wild-type HIV).

The crHIV vectors conditionally replicate due to the fact that they contain the sequences required for RNA expression, dimerization and packaging, but do not express functional (i.e., wild-type) HIV-1 proteins. A selective advantage was imparted to the crHIV vectors by inserting a ribozyme cassette that cleaves in the U5 region of the wild-type HIV genome, but not the crHIV U5 RNA.

The ribozymes present in the vectors do not cleave the crHIV RNA because the U5 region of the crHIV RNA has been modified by conserved base substitution (base substitutions present in other HIV strains) to prevent the ribozymes from efficiently binding and cleaving these sites. Moreover, the crHIVs are nonpathogenic because they do not code for proteins believed to be responsible for CD4+ cell death. When the HIV-infected cells (that have been transfected with the crHIV vector) become activated, the cells become capable of complementing the crHIV genomic deficits, resulting in the production of crHIV progeny virions.

In general, crHIV genomes were constructed from the full-length, infectious HIV clone, pNL4-3 (Adachi et al. (1986), supra. All cloning reactions and DNA, RNA, and protein manipulations were carried out using methods well known to the ordinary skilled artisan, and which have been described in the art, e.g., Maniatis et al., Molecular Cloning: A Laboratory Manual, 2nd ed. (Cold Spring Harbor Laboratory, NY (1982)). Enzymes and reagents employed in these reactions were obtained from commercial suppliers (e.g., New England Biolabs, Inc., Beverly, Mass.; Clontech, Palo Alto, Calif.; and Boehringer Mannheim, Inc., Indianapolis, Ind.) and were used according to the manufacturers' recommendations. Moreover, vector maintenance and propagation were done using techniques that are commonly known, and that have been described previously (e.g., Dropulic' et al. (1992), supra; and Dropulic' et al. (1993), supra). pNL4-3 was cleaved with the enzymes Pst I (which cleaves in gag, at about position+1000 from the start of transcription) and Xho I (which cleaves in nef, at about position+8400 from the start of transcription), and a polylinker containing convenient restriction sites was inserted. A 0.86 kb Bgl II to Bam HI fragment containing the rev responsive element (RRE) was cloned into a Bam HI site present in the polylinker. These manipulations resulted in deletion of the HIV wild-type genome from within the gag coding region to within the U3 coding region (i.e., thus also deleting the nef gene). While the vector is able to produce a truncated gag transcript, a full-length functional Gag protein is not produced by the vector. However, inasmuch as wild-type Gag functions are unnecessary according to the invention, the gag sequences can be mutated to prevent Gag protein from being translated.

A ribozyme cassette containing either single or multiple ribozymes as described herein was inserted into a Sal I site downstream from the Bam HI site. To accomplish this, complementary deoxyoligonucleotides encoding ribozyme sequences were synthesized, annealed and then cloned into the Sal I site. The ribozymes employed for construction of the crHIV vectors were hammerhead ribozymes. These ribozymes contained a catalytic domain comprised of 22 base pairs, and two hybridization domains comprised of 9 base pairs each. The ribozymes were targeted either to the +115 or +133 site (i.e., corresponding to the number of base pairs downstream from the start of transcription) of the U5 RNA sequence. The hybridization domains and catalytic domain (underlined) of the ribozymes targeted to the +115 site and the +133 site are as follows:

| | | |
|---|---|---|
| CACACAACACTGATGAGGCCGAAAGGCCGAAACGGGCACA | (“the +115 ribozyme”) | SEQ ID NO:3 |
| ATCTCTAGTCTGATGAGGCCGAAAGGCCGAAACCAGAGTC | (“the +133 ribozyme”) | SEQ ID NO:4 |

The ribozyme cassette was comprised of either a single, double or triple ribozyme(s) placed in tandem. Vectors containing either single or triple ribozymes may be readily constructed and targeted to the same site of the U5 HIV RNA, at position +115. Vectors containing double ribozymes may be readily constructed and targeted either to the same site at position +115 or to different sites at positions +115 and +133 of the U5 HIV RNA.

To complete the construction of the vectors, the crHIV vectors were rendered resistant to ribozyme cleavage (i.e., in their manifestation as RNA) by mutating a site recognized by the hammerhead ribozymes occurring within the U5 region of the crHIV genome. To accomplish this, a double-stranded oligonucleotide (i.e., AAGCTTGCCTTGAGTGCTCAAAGTAGTGTGTGCCCACCTGTTGTGTGACTCTGGCAG CTAGAGATCCCACAGACCCTTTTAGTCAGTGTGGAAAATCTCTAGCAGTGGCGCC SEQ ID NO:13) containing the base substitutions depicted in FIG. 2 SEQ ID NO:2 was used to introduce modified sites into the vector. Specifically, base substitutions were engineered into the ribozyme hybridization and cleavage sites at base pairs 115 and 133. In particular, as illustrated in FIG. 2; mutations were introduced at base pairs 113, 114, 132, 134 and 142. These sites can be modified to comprise any mutation (i.e., GTGTGCCCNNCTGTTGTGTGACTCTGGNANCTAGAGANC, wherein N can be any mutant nucleotide SEQ ID NO:14). Preferably, however, the sequences are mutated such that there is, for instance, a G to A substitution at site +113 (i.e., such that the sequence comprises GTGTGCCCATCTGTTGTGTGACTCTGGTAACTAGAGATC SEQ ID NO:15), a U (i.e., T, in terms of the DNA sequence) to C substitution at site +114 SEQ ID NO:5, a U (i.e., T, in terms of the DNA sequence) to C substitution at site +132 SEQ ID NO:6, an A to G substitution at site +134 (i.e., such that the sequence comprises GTGTGCCCGTCTGTTGTGTGACTCTGGTAGCTAGAGATC SEQ ID NO:16) and a U (i.e., T, in terms of the DNA sequence) to A substitution at site +142, which mutations can be made either alone, or in combination. In particular, in the absence of other U5 mutations, the U (i.e. T, in terms of the DNA sequence) to C substitution at site +114 SEQ ID NO:5 and/or site +132 SEQ ID NO:6 in the crHIV US RNA prevents its scission by ribozymes (Uhlenbeck (1987), supra). The inserted base-substitutions are present in various other strains of HIV (Myers et al., HIV Sequence Database, Los Alamos Nat. Lab. (1994)), which indicates that these substitutions do not decrease the replicative capacity of the HIV genome.

The method as set forth herein can be employed to construct other conditionally replicating vectors, for instance, comprised of differing viral genomes (e.g., different RNA viruses), or comprised of different genetic antiviral agents. Furthermore, a conditionally replicating vector can be further modified to impart to a host cell, into which the vector is introduced, a selective advantage over a host cell containing the wild-type virus. For instance, such a vector can be modified to further encode multidrug resistance, or a mutated protease or reverse transcriptase.

These methods can of course can also be employed to construct the various helper vector constructs described herein.

### Example 2

This example describes the resistance to ribozyme cleavage of conditionally replicating vectors, and, in particular, of the crHIV vectors.

To confirm the resistance to ribozyme cleavage of the crHIV vectors, in vitro transcription was performed. To accomplish this, the ribozyme sequences were cloned into the Xho I site of pBluescript KSII (Stratagene, La Jolla, Calif.). A 0.21 kilobase pair (kb) Bgl II fragment containing the mutated crHIV U5 region similarly was excised from the crHIV vector and inserted into the Bam HI site of pBluescript KSII. The resultant modified pBluescript KSII vectors were linearized with Bss HII prior to in vitro transcription. A similar plasmid expressing wild-type HIV U5 RNA (described in Myers et al. (1994), supra) was employed as a control. It was linearized with Eco RI prior to in vitro transcription.

Radiolabeled U5 HIV RNA and ribozyme RNA were produced by in vitro transcription of the vectors, as previously described (Dropulic' et al. (1992), supra). The radiolabeled transcripts were incubated together (at a target to ribozyme molar ratio of 1:2) in 1×transcription buffer containing 40 mM Tris-HCl, pH 7.5, 6 mM MgCl₂, 2 mM Spermidine, and 10 mM NaCl. The samples were heated to 65° C., and then cooled to 37° C. for 5 min prior to the addition of stop buffer solution containing 95% formamide, 20 mM EDTA, 0.05% Bromophenol Blue, and 0.05% Xylene Cyanol FF. The products' were then resolved by denaturing polyacrylamide gel electrophoresis (PAGE), and detected by autoradiography.

When wild-type U5-HIV RNA was incubated with a transcript containing a single ribozyme to site +115, cleavage was readily observed. Such cleavage results in products P1 and P2. Cleavage also can be seen when wild-type HIV RNA was incubated with RNAs containing double ribozymes to either the same site, or to different sites. When a ribozyme-containing transcript directed to two different sites was incubated with wild-type HIV RNA, products P1, P2 and P3 were produced. P3 results from cleavage at the +133 site.

In comparison, when the modified U5-containing crHIV RNA was incubated with either a single ribozyme directed to the +115 site, or double ribozyme directed to either the +115 site or the +133 site, cleavage products were not detected. Thus, these results confirm that crHIV U5 RNAs are resistant to ribozyme cleavage, while wild-type HIV-U5 RNAs are cleaved by anti-US ribozymes. Moreover, the results validate that the approach of the present invention can be employed to impart conditionally replicating vectors (including vectors other than crHIV vectors) with a selective advantage for replication when introduced into a host cell as compared with a wild-type strain of virus.

### Example 3

This example briefly describes key steps in the production of helper vector constructs of the invention. Unless otherwise noted, nucleic acid sequences encoding various retroviral elements were obtained from publicly available sources. All described steps required sequencing of the whole coding region for all proteins to detect mistakes and direct corrections if necessary.

To degenerate the tat sequence and permit its targeting by anti-tat ribozymes, plasmid pTAT was mutagenized using QuickChange kit from Stratagene to include targeting sites for an anti-tat ribozyme cassette. Subsequently, the mutagenized tat sequence was cloned into an expression vector based on pMG plasmid from InvitroGen. The tat sequence was fused to IRES at the first ATG using an NcoI site. The vector was further modified to contain an ampicillin resistance gene and SV40 origin of replication.

Sequences containing the HIV-1 rev and RRE elements were obtained by excising the third exon of rev together with the RRE from pNL4-3, and cloned into the pLITMUS38 plasmid. Rev sequences up to nucleotide 208, which includes the second exon and part of the third exon up to a BamHI site, was assembled from 3 synthetic nucleotides using ETR and PCR. The PCR product was cloned into pUC18, and than subcloned into pLIT/RRErev3. The two pieces of rev were fused using QuickChange kit. RRE from HIV-2 was substituted for the HIV-1 RRE and both constructs were cloned into a gag/pol/Rz vector. Degenerated rev sequences were also used directly for subcloning into packaging construct for vectors such as pVIRPac-2.

To degenerate the first 42 nucleotides of gag, which are required for packaging and must be a part of the vector plasmid, a BssHII/SpeI fragment from pNL4-3 was subcloned into pLITMUS38. The gag sequence was degenerated using QuickChange kit so that all components of the packaging signal were eliminated, and an HIV major splice donor was reinserted in front of the first ATG codon of gag. The degenerated gag was subcloned into the pNgp plasmid, courtesy of Dr. Conde, in place of 5′ LTR to form a continuous gag sequence. This resulting construct still contains splice acceptor (SA) and splice donor (SD) sequences utilized to express the vif protein. This splicing may result in the presence of ribozyme cassette in spliced mRNA, which is undesirable due to possible cleavage of the spliced retroviral (conditionally replicating) vector RNA and resultant decrease in the titer of packaged transducing vector. Overlapping PCR was used to mutate the SA and SD sequences. The PCR product was inserted cloned into a gag/pol construct, followed by insertion of an anti-U5 ribozyme cassette and the rev and RRE elements.

The vesicular stomatitis protein G (VSV-G) env protein was cloned into the first MCS of a pMG-tat vector to generate an envelope expression cassette. To simplify further cloning steps VSV-G was cloned as a blunt ended fragment into the Bgl II site filled in with Klenow polymerase. A construct with the SV40 origin of replication (ori) was generated. To finalize packaging plasmids as helper vectors, a rev sequence was subcloned into the second MCS. The resulting plasmid was called pVIRPAC-2 (see FIG. 6).

To generate helper vectors from some packaging plasmids, the gag/pol/Rz/RRE/rev cassette was subcloned into pMG-tat-G plasmid (without an SV40 ori). Constructs with an RRE from either HIV-1 or HIV-2 were made (pVIRPac-1.1 and 1.2Rz in FIG. 6). As a control for testing ribozyme function, the ribozyme cassette was deleted in the final HIV-2 RRE containing construct, generating pVIRPac-1.2. Finally, to test the hypothesis that forcing gag/pol RNA into the splicing machinery may prevent cleavage of retroviral (conditionally replicating) vector genomic RNA, the intron from β-globin was inserted in front of the SV40 poly A signal (pVIRPac-1.2RzIn). Of course in other embodiments of the invention, the inserted intron is derived from HIV, preferably a complete HIV intron confirmed to direct an RNA into the cellular splicing machinery (e.g. spliceosome).

### Example 4

This example describes the efficacy of various helper vector constructs. Standardized protocols for retroviral vector production by transient transfection was used in these experiments. Megapreps of plasmid DNA were produced using known techniques. To produce the virus, 293T cells were co-transfected with retroviral vector and helper vector plasmids by calcium phosphate method. Cell supernatants were collected approximately 40 h after transfection, filtered through 0.45 μm syringe filters, and titrated on HT1080 cells. Vector titer was calculated based on its transduction efficiency measured by flow cytometry approximately 72 h after infection. The arbitrary formula was used for titer calculations was N*400,000*, where N is the fraction of transduced cells; 400,000 is the approximate number of cells at the time of infection; and F is a dilution factor.

To enhance the safety of a helper vector by decreasing the possibility of generating RCV upon recombination with an HIV-1 based retroviral vector, an anti-U5 ribozyme cassette was inserted into certain helper vector constructs. The ribozyme would cut and destroy retroviral vector RNA if co-packaged into the virion. It is possible, however, that the ribozyme would also cleave vector RNA inside the producer cells, thus interfering with virus production. To further increase safety, the helper constructs contained the RRE-2 element to further decrease the likelihood of helper and vector being co-packaged into a viral particle. These helpers were used to package the pN1(cPT)GFP vector.

As shown in FIG. 7, the presence of a ribozyme on the helper construct had little effect on vector titer. Importantly, a helper construct (pVirPac 1.2RzIn) containing an intron to affect the cellular trafficking of helper RNA away from vector RNA also had no significant effect on vector titer. The vector titer generated with this 2-plasmid system is comparable to the titer obtained using conventional 3-plasmid transfection systems Similar experiments with an RRE1 containing helper construct, pVirPac1.1, showed about a 5-fold lower packaging efficiency. Thus, incorporation of RRE-2 into the helper vector construct not only increased its safety by reducing the possibility of homologous recombination, but also unexpectedly increased efficiency of packaging.

### Example 5

This example tests whether expression of the MGMT gene from the HIV-LTR spliced mRNA is sufficient for efficient selection of MGMT transduced cells with BG and BCNU. FIG. 10A shows the vectors used. FIG. 10B shows effective cell survival and expansion in the presence of cytocidal concentrations of BG/BCNU. The letter designations are as follows: M, MGMT; I, IRES;G, GFP; W, Woodchuck post-transcriptional element; and C, CMV promoter. All of the above elements were cloned into a pN1 vector, downstream of the RRE element such that any gene directly 3′ to the RRE element would be expressed from the spliced mRNA since the splice acceptor site was located just proximal to the gene insertion site. The figure demonstrates that while control cells not transduced with a lentiviral vector (“CGFP”) did not expand or survive, all vectors expressing the MGMT did survive and expand.

Cells transduced with pN1CMIG were dead after 14 weeks while cells transduced with pN1MCG were dead after 21 weeks. Importantly, cells transduced with pN1MIG and pN1MIG-W remained alive after 29 weeks, indicating long term survival of cells transduced with a vector without an inserted internal promoter.

Surprisingly, vectors expressing MGMT from the HIV-LTR spliced mRNA were selected very efficiently. The expression of MGMT does not limit the scope of payload genes either to MGMT or selection genes. These results indicate that any gene can be expressed from the HIV-LTR promoter in T cells. Similar data has been obtained in non-T cells, demonstrating that the expression of genes from HIV-LTR spliced mRNA could be applied for the expression of genes in many cell types.

Additionally, the bioistronic nature of the “MIG” constructs demonstrates that multiple genes can be expressed when linked by an IRES. Examples beyond the above include MGMT or other selection gene with a chemokine, interferon, or other genetic antiviral agent.

### Example 6

Human CD4+ T cells were tested for their sensitivity to BG and BCNU. CD14 depleted CD4+ cells were purified from peripheral blood with or without cytokine mobilization. The cells were cultured in 3 μg/ml PHA and 5 ng/ml IL-2 for 4-7 days prior to BG and BCNU treatment. Under appropriate conditions in the absence of BFG and BCNU, CD4+ cells can be expanded up to 100 fold over 10 days in culture. Doses tested were 0, 0.2, 0.5, 2, 5, 10, 20, 30, and 40 μM BG and 0, 2, 5, 10, 20, 30, and 40 μM BCNU. A series of three experiments were performed.

FIG. 11 shows representative results from the above. Expansion was reduced to 20-fold after 10 μM BG and 1M BCNU, and to approximately 10-fold or less after 20 μM or more of BCNU. Treatment of CD4+ cells with BCNU did not result in growth delay. Thus, unlike the SupT1 cells used in the above example, CD4+ cells are not sensitive to low dose BCNU alone. This suggests greater MGMT expression primary CD4+ cells, although variation in MGMT expression is expected based on variation among T cell donors.

The presence of both BG and BCNU, each at 10 μM or higher, was best at sensitizing CD4+ cells, while BG at 0.5 μM and 15 μM or more of BCNU similarly sensitized the cells. Increasing the BG dose did not increase cell killing, although toxicity to CD4 cells increased with the BCNU dose.

As shown in FIG. 11, the level of selection generally increased 5-fold from baseline 20% to 100%. In one experiment, selection was from 3% to >90%. 10-100 fold expansion was seen after selection and one week; 20-400 fold expansion was seen in 2.5 fold, with an average of 80-fold.

### Example 7

Previous work suggests a correlation between the purging of bone marrow autografts with improved disease free survival after autologous transplantation. Also, the purging of normal T cells from an allograft has been used to decrease the risk of graft-versus-host disease. FIG. 13A demonstrates the efficiency of transducing tumor cells with a single round of transduction using pN1(cPT)ASenvGFP. FIG. 13B confirms that tumor cells may be selectively and effectively killed over CD34+ stem cells since the latter show no significant transduction after a single round of transduction.

The application of the above to the purging of tumor cells from an autologous bone marrow graft begins with harvesting bone marrow or collecting peripheral blood stem cells. CD34+ cells are purified via a CD34 antibody column and then transduced with a conditionally replicating vector at an MOI of 2 to 400 for 2 to 16 hours. The cells are optionally incubated in the presence of one or more cell activating factors, such as cytokines, to either assist transduction or maintain the cells. The cells are then transferred into the subject being treated.

The ability to similarly target T cells in an allograft uses conditionally replicating vectors, in combination with a high efficiency stable transduction protocol like that in co-pending U.S. patent application Ser. No. ______ (yet to be assigned) filed Aug. 31, 2000 as attorney docket no. 397272000400, to selectively transduce T cells.

### Example 8

FIG. 14, panel B, shows the effective pseudotyping of HIV-1 vectors of the invention with various envelope proteins.

Similar initial titers with a Lentiviral vector pseudotyped with VSV-G envelope protein can be effectively increased via purification using either high speed centrifugation or diafiltration. Vector pN1(cPT2)ASenvGFP was packaged with pVirPac1.2Rz2. The table below shows (a) the total protein in a sample, (b) the vector titer in the sample and (c) the fold purification of the vector from extraneous material in the sample.

| | Total Protein, | Vector Titer, | |
|---|---|---|---|
| Process Step | mg | TU/ml | Purification (fold) |
| Start after clarification | 3207 | 5.9 × 10⁶ | N.A. |
| Concentration | 722 | 8.2 × 10⁷ | 61.7 |
| Diafiltration | 224 | 6.8 × 10⁸ | 1650 |

As seen from the table, the total protein in the vector supernatant sample after clarification of cell debris is 3027 mg, while the vector titer at this stage is 5.9×10⁶. The sample then undergoes concentration by ultrafiltration by which time the total protein in this sample is 722 mg and the vector titer increases to 8.2×10⁷, a 61.7 fold purification. After concentration by ultrafiltration the sample is diafiltrated by which time the total protein in the sample has further decreased to 224 mg while the vector titer has increased to 6.8×10⁸ per ml, a 1650 fold purification. This demonstrates that non-vector extraneous protein can be removed and Lentiviral vector that is pseudotyped with VSV-G can be purified using the above described protocol.

The above may also be used in combination with vectors produced in a Hela-tat cell line generating titers of approximately 10¹⁰ as shown in FIG. 16.

### Example 9

Alternative helper constructs for pseudotyping conditionally replicating vectors have been developed by placing VSV-G expression under Rev-dependent control. Since RRE-containing mRNAs are not expressed in the absence of Rev, it has been postulated that they are defective due to the presence of the cis repressive sequences (CRS) residing within the gag/pol, pol and env genes. The CRS has been reported to trap mRNA in the nucleus, destabilize mRNA, or prevent mRNA association with ribosomes. Thus the presence of the CRS and Rev supplied in trans, combined with mRNA splicing, may be used to produce to regulate gene expression.

The Rev/RRE/CRS system was used to control VSV-G expression in a Rev-dependent manner. Helper constructs for this approach contain a 5′ splice donor site; an HIV-2 RRE and a 3′ splice acceptor site (preferably for a tat/rev); a CRS fragment from the HIV-2 pol region to decrease the likelihood of recombination with HIV-1 based vectors; and optionally a non-inducible promoter, such as the CMV immediate early (IE) promoter, instead of any inducible promoters. VSV-G encoding RNAs, while produced without induction is still regulated by the presence of Rev since only unspliced RNAs may be exported and expressed in the cytoplasm without Rev. VSV-G expression remains Rev mediated because Rev counteracts the nuclear retention of the transcripts controlled by mRNA splicing and CRS.

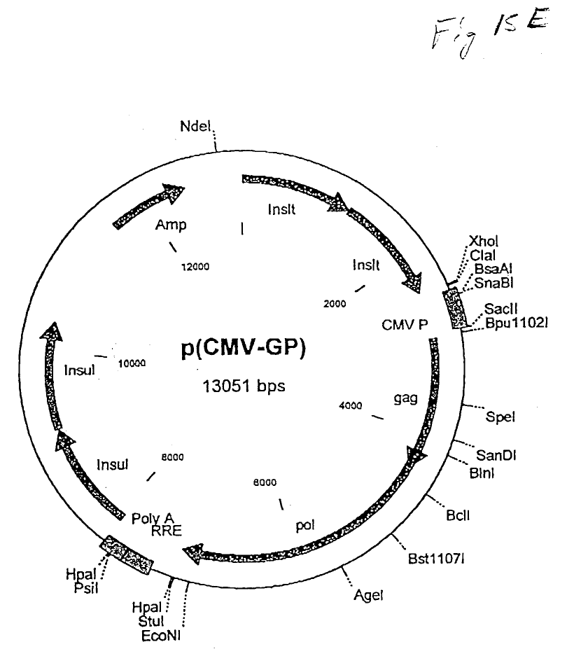

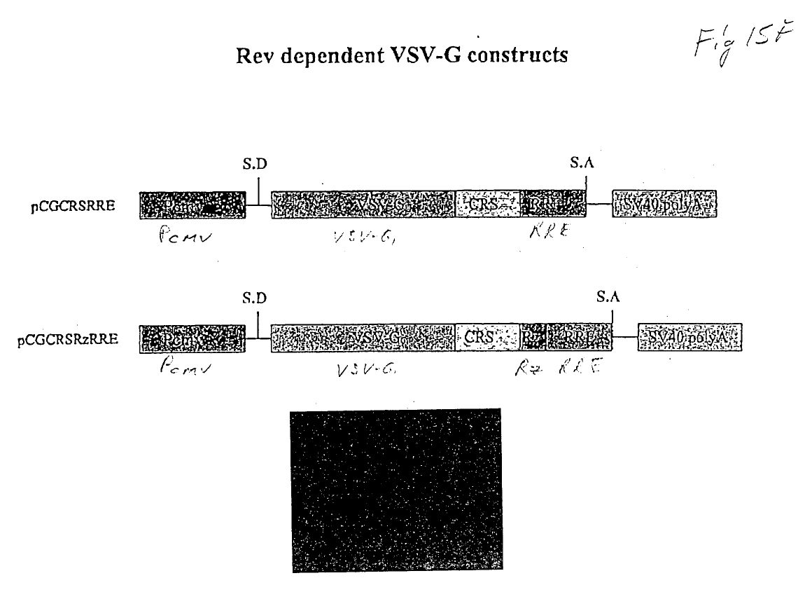

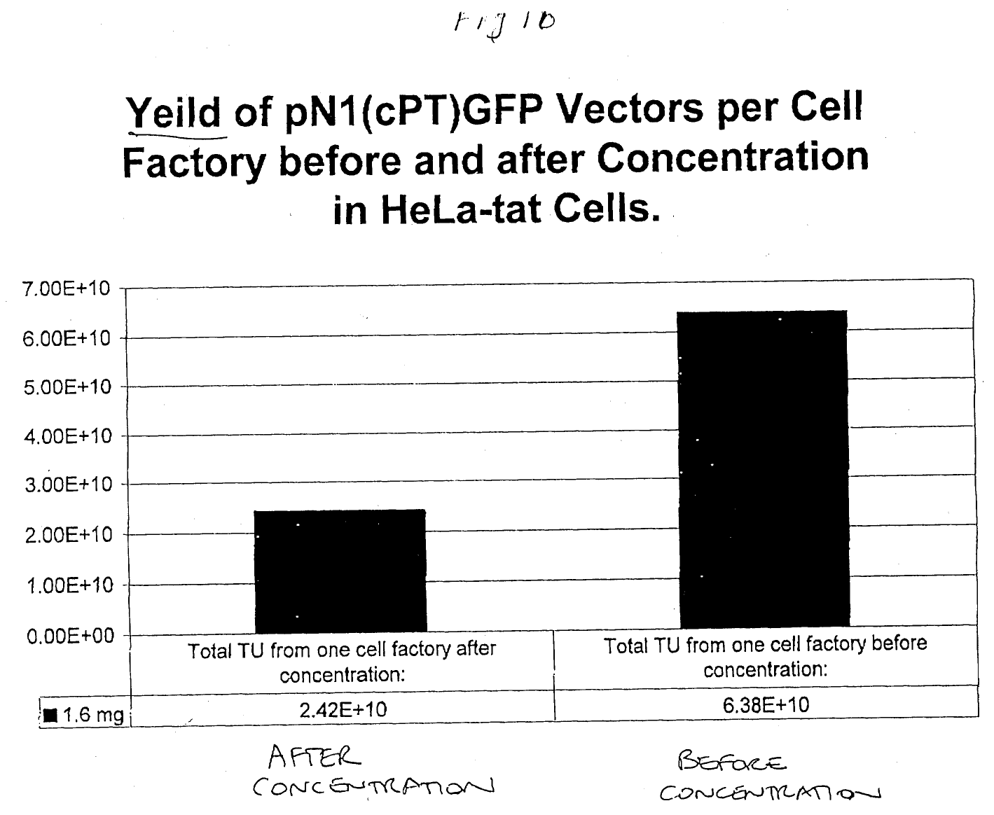

Preferably, the splice donor site is not a synthetic 5′ β-globin splice donor site but a native HIV, or HIV analog, splice donor site. The HIV-2 RRE is a 280 bp fragment containing both the HIV-2 RRE and the splice acceptor site for Tat/Rev; and the CRS is a 550 bp internal fragment from the HIV-2 pol sequence. The CRS and RRE elements from HIV-2 may be prepared by PCR. Non-limiting examples of such helper constructs include pCGCRSRRE, pCGCRSRzRRE, and others shown in FIGS. 15A-15E. The final plasmid constructs were confirmed by multiple restriction enzyme digestion. While pEH-GP and pCMV-GP are constructs that constitutively express gag-pol polyprotein, other promoters, including a lentiviral or HIV promoter, may be used if desired.

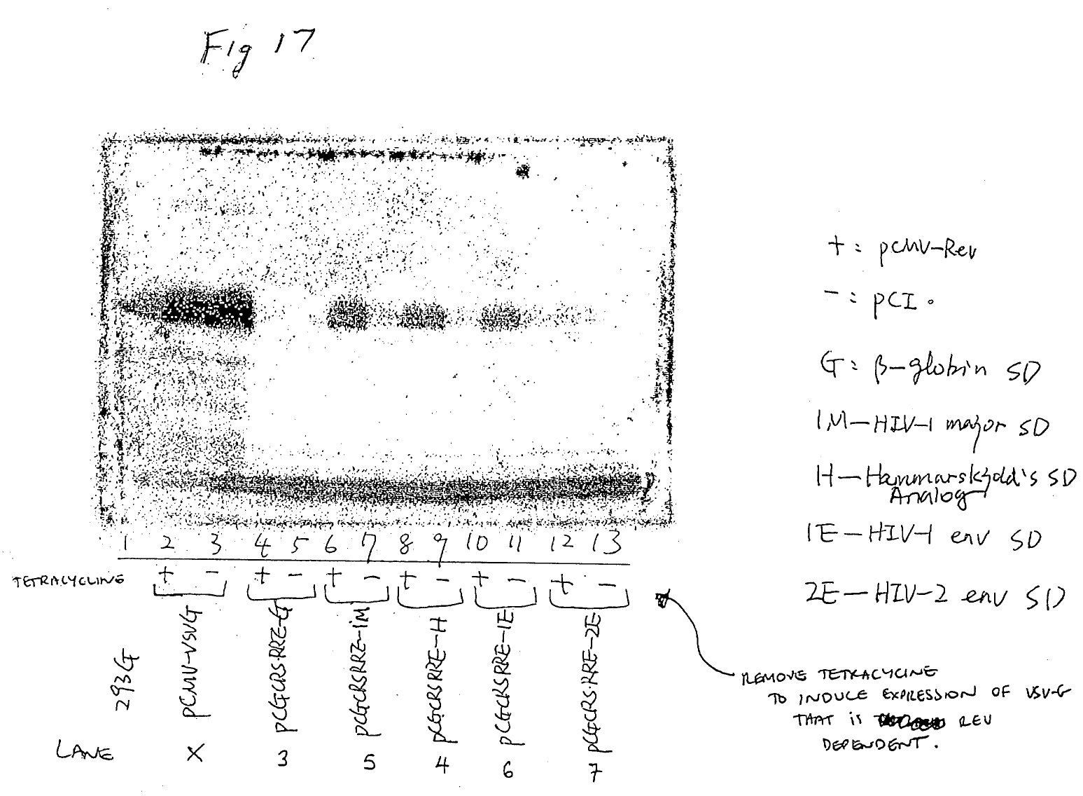

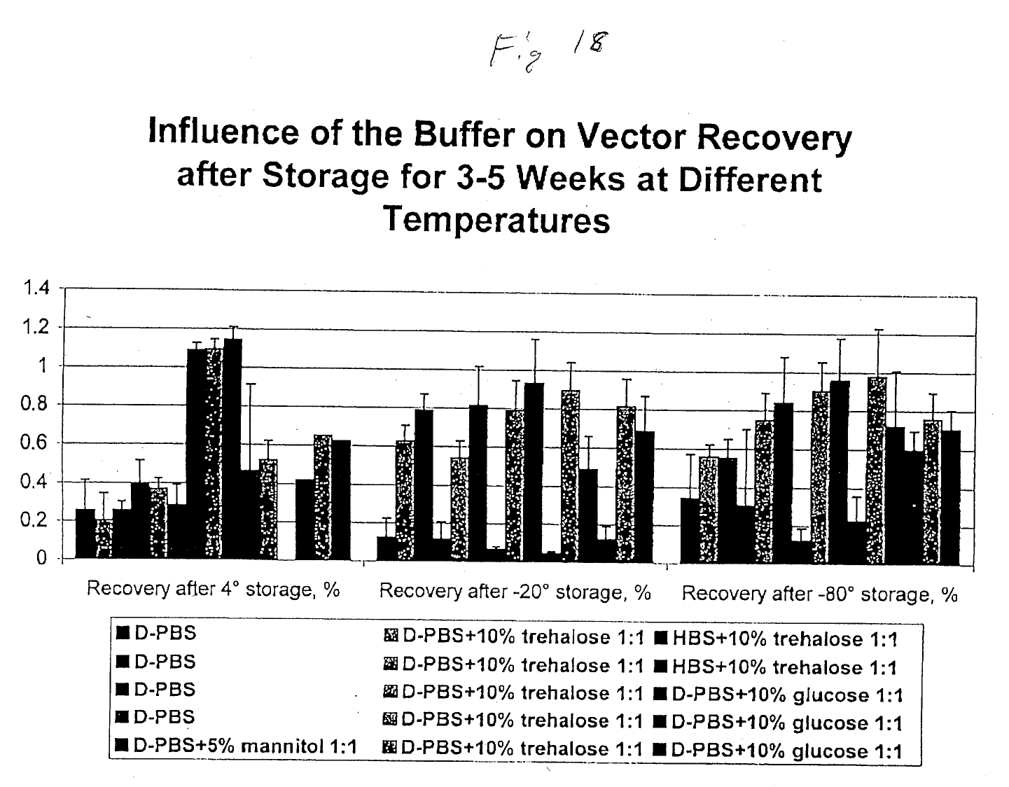

FIG. 17 shows the results from using a number of different splice donor combinations for Rev dependent expression of VSV-G. As shown, removal of tetracycline results in Rev dependent expression.

Additional examples of splice-donor site combinations, as well as a concensus sequence, are provided below. While all may be used, the HIV major, HIV-1 env, HIV-2 major, and analog splice-donor combinations are preferred.

| | |
|---|---|
| CONCENSUS SPLICE DONOR: | NNNNAGGTAAGTNNN |
| BETA-GLOBIN SPLICE DONOR: | NGGGCAGGTAAGTAT |
| HIV MAJOR SPLICE DONOR: | NNGACTGGTGAGTAN |
| HTV-1 ENV SPLICE DONOR: | AAAGCAGTAAGTAGT |
| HIV-2 ENV SPLICE DONOR: | AGACAAGTGAGTAAG |
| HIV-2 MAJOR SPLICE DONOR: | NNGAAGGTAAGTGCN |
| ANALOG SPLICE DONOR: | CTTCAGGGTGAGTTNN |

### Example 10

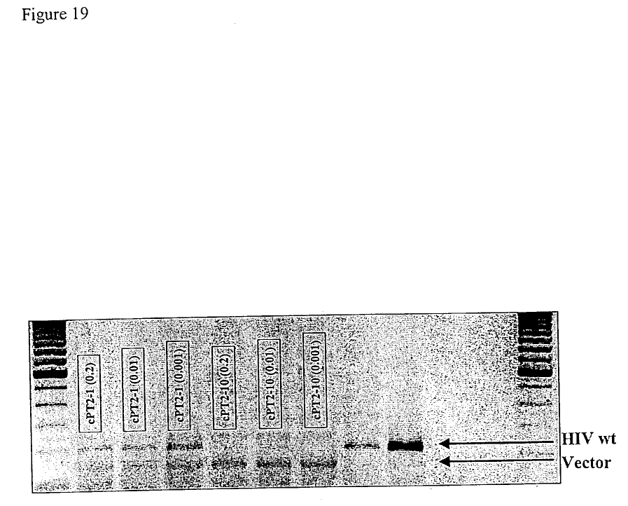

To identify appropriate conditions for the storage of vectors prior to use, a variety of storage buffers and conditions were tested. The results are shown in FIG. 18. The ability to store vectors at −20° C. and in a pharmaceutically acceptable buffer without significant loss of vector recovery will be of tremendous advantage in maintaining vectors prior to use. Of course the storage of vectors at about −80° C., about −20° C., and about 4° C. in other pharmaceutically acceptable compositions may also be readily practiced.

### Example 11

This example describes the amino acid sequence of a chimeric HIV CTL epitope for use in the practice of the invention. The sequence contains a first methionine (M) to initiate translation followed by various contiguous subsequences corresponding to p17, p24, p 15, Pol, Rev, gp120env, gp41 env, and nef, respectively.

M-
KIRLRPGGNKKYKLKHIVWASRELERFGSEELRSLYNTVAVLYCVHQKIE
VKDTKEALDTENRNQESQNY-
PIVQNLGQMVHQALSPRTLNAWVKVIEEKAFSPEVIPMFSAISEGATPQD
LNTMLNTVGGHQAAMQMLKATINEEAAEWDRLHPVHAGPIAPGQMREPRG
TSTLQEQIAWMTNNPPIPVGEIYKRWIILGLNKIVRMYSPVSIFRDYVDR
FYKTLRAEQATQEVKNWMTETLLVQNANPDCKTILKAILEDMMTACQGVG
GPGIKLKARLV-
QEGHQMKDCTERQANFGNFPQSRLEPTAPPE-
ITLWQRPLVDTVLEDMNLVLVGPTPVNJSPJETVPVKLGPKVKQWPLALV
EICTEMEKIEGKISKIGPTVLDVGDAYFSVPLDEDFRKYTAFTIPSIWKG
SPAIFQSSMTKNPDIVIYQYMDDLYVDLEEGQHRTKIEELRQHLLRWGFT
TPDKKPIKLPEKESWLVGKLNWASQIYAGIKVKQLIPITEEABLEILKIP
VHGVYQIYQEPFKNLKTGDVKQLTEAVKITTESIVIWPIQKETWETWWTE
YWPLVKLWYQLEPIVGAETFYVDGAANKALQDSGLEVNIVTDSQYALGIE
SELVSQIIEQLLAWVPAHKGYEEAEVIETAYFILKLLLWKGEGAV-
ISGWILNTY-
RVKGIRKNYAENLWVTVYYGVPVWKEATTTLFCASDAKAYDPNPQEVVLH
EDIISLWDQSLKKLTPLCVTLNCSFNVTTLINTSYTLINCKSSTITQACP
KCKNVSTVQCRPVVSTQLLLNGSLAEEDIVSIEINCTRPNNNTRIKKITL
GPGRVLYTTGENNTLKQIVEKLREIKQFKPEIVMHSFNCGGEFFYCNSTQ
LFLPCRIKQIINRWQEVGKAMYAPPIEGQIRCLSNITGVKIEPLGVAPTK
ARRRVVQR-
RAIEAQQHLGIKQLQARVLAVERYLKIDQQLLGITTVPWNASWWYIKIHM
IVGVLSIVNRVRQGYSPLSFQTHRLVDGFLTLLYHRLIDLLLIAKRGRRG
WAALKYSLLNATAIAVDRVIEIVQRTCRAILHIPRRIRQGLERALL-
WPAIRERMVGFPVRPQVPLRPMTYKAAHDLSHFLKEKGGLEGLIYSQKRQ
DILDLWVYHTQGFFPDWQNYTPGPGTRYPLCFGWCFKLVPVVLMWKFDSK
LAFHHVARELHPEYFKDC-

Example 12 Dose dependent inhibition of wt-HIV DNA formation in primary CD4+ T cells was observed after prior introduction of an HIV based vector (see FIG. 19). Primary CD4+ T cells were transduced with vector and cultured for 5 days under stimulatory conditions before challenged with wt-HIV infection. The HIV based vector (cPT2) at multiplicities of infection of 1 or 10 (designated in the figure as cPT2-1 or cPT2-10) were used. The transduced cells were then challenged with wt-HIV (NL4-3 strain) at varying multiplicities of infection (0.2, 0.01 and 0.001). The last two lanes of FIG. 19 are pNL4-3 plasmid spiked DNA positive controls showing wt-HIV DNA amplification. The cells were lysed on day 8 after infection and DNA extracted using established methods. DNA PCR was performed on the extracted cellular DNA samples using primers specific for wt-HIV and vector sequences to discriminate between integrated wt-HIV and vector DNA. The wt-HIV specific primers are directed to the nef gene sequences which are absent in the vector used. The data shows that while at a vector transducing MOI of 1 (cPT2-1) the cells are permissive to wt-HIV DNA formation, as shown by a visible signal of wt-HIV DNA in DNA samples, no wt-HIV DNA formation is seen at a vector MOI of 10 (cPT-2-10). This indicates that the vector can inhibit the production of wt-HIV DNA and prevent its integration into the transduced host cell. The data also indicates that the level of inhibition is dose dependent.

### Example 13

This example provides a summary of the embodiments of the invention as described above. The invention includes a conditionally replicating viral vector comprising a gene (or other nucleic acid) to be expressed and one or more first nucleotide sequences wherein the vector (a) replicates in a host cell only upon complementation with a wild-type strain of virus, a helper virus, or a helper vector, each of which is sensitive to the presence of said one or more first nucleotide sequences; (b) is resistant to the presence of said one or more first nucleotide sequences; and (c) contains one or more substitutions, insertions, or deletions that decrease the likelihood of generating a replication competent vector.

To decrease the likelihood of generating a replication competent vector, the invention provides vectors containing degenerated sequences with respect to one or more sequences of a helper construct, of a host cell's nucleic acid content, or of other nucleic acids. Additionally, helper constructs for use in combination with such vectors for their packaging. Such helper constructs may also contain degenerated sequences with respect to one or more sequences of the vector, of a host cell's nucleic acid content, or of other nucleic acids.

The vectors of the invention may also contain one or more sequences that target a helper construct. Such sequences include genetic antiviral agents, and preferably are (or encode) ribozymes or antisense nucleic acids. Preferred ribozymes and antisense sequences are able to undergo basepair complementarity with their respective targets via G-U base pairing in addition to standard A-T, A-U, and G-C base pairing. Similarly, the helper constructs of the invention may also contain the same type of sequences to target the vector.

Helper vectors of the invention may also contain one or more sequences that affect the localization, or cellular tracking of RNAs expressed from by the helper in comparison to a vector. For example, the helper vectors may contain an intron, a heterologous RRE in comparison to the vector, or a heterologous gag sequence in comparison to the vector. The helper vectors may also contain a degenerated gag and/or pol sequence(s) in comparison to the vector such that helper and vector RNAs are less likely to co-localize. The helper vectors may also contain splice-donor sites such that packaging and RRE signals (sequences) are removed from RNAs to prevent their co-localization with vector RNAs. Such splice-donor sites may also be present in helper vectors to result in the differential tracking of a vector in comparison'to a helper vector or wild-type viruses.

Helper vectors of the invention may also be capable of pseudotyping the vectors of the invention. Exemplary examples include the VSV-G, RD114, rabies virus, GALV, and chimeric envelope proteins. Helper vectors of the invention are also preferably derived from a heterologous virus with respect to the vector. An example is helper vectors derived from HIV-2. Methods of the invention include the use of such helper vectors to replicate vectors of the invention.

Exemplary vectors of the invention include those of the pN1 and pN2 series shown in FIGS. 1A to 1K, especially with the removal or substitution of the GFP sequences. Preferably, the vectors retain at least the antisense sequence and the cPT containing sequence. Exemplary helper constructs of the invention include those-of the pVirPac series as shown in FIGS. 6A-6G with and without ribozymes.

All vectors and helpers of the invention may be degenerated as described above or in other parts of the constructs as necessary. Preferred degeneracy is to codons preferentially used in human cells. The vectors and helpers may also be chimeric in nature such that they are derived from different naturally occurring as well as synthetic nucleic acids. Preferred chimeric constructs contain both HIV-1 and HIV-2 sequences. Especially preferred vectors, however, are HIV-1 derived or encode an HIV-1 Tat protein. Alternatively, the vectors do not encode a Tat protein, which is supplied by a helper vector, a wild-type virus, or a host cell.

The vectors of the invention also preferably do not encode or do not express viral accessory proteins. The vectors may also comprise a nucleic acid that diminishes, minimizes, or prevents viral infection when present in a host cell or renders said cell resistant to toxic agents. Examples of such nucleic acids include a cell surface protein or a truncated CCR5 protein, preferably the CCR5Δ32T protein. Other examples include nucleic acids encoding resistance to a drug or DNA damaging agent, preferably multidrug resistance or an MGMT variant, especially the G156A variant. Additional examples include nucleic acids encoding a transdominant phenotype. An exemplary example is a transdominant HIV-1 gag sequence.

The vectors of the invention may also contain sequences to assist the expression of a heterologous sequence. An exemplary example is an insulator sequence. The vectors may also contain a sequence that improves transduction of the vector into cells. An exemplary example is the 545 basepair sequence containing the cPT described herein, although smaller fragments or larger fragments containing the cPT may also be used. The vectors may also contain LTRs to express genes as well as nucleic acids, including LTRs modified in the transcription binding sites located therein. Preferably, such modified LTRs are contained in Lentiviral vectors. Methods of the invention include the use of such vectors.

The vectors of the invention may also express at least two genes using appropriately placed splice sites or IRES elements. The vectors may also be packaged into viral particles with one or more chimeric envelope proteins which stimulate target or host cells for transduction. Additionally, the vectors may be packaged in cells where the helper is a protein or the helper contains an Rev/RRE/CRS system for regulating gene expression. Methods of the invention include the use of such vectors.

All of the references cited herein, including patents, patent applications, and publications, are hereby incorporated in their entireties by reference. As used herein, all terms presented in the singular form are intended to include both the singular and plural forms.

While this invention has been described with an emphasis upon preferred embodiments, it will be apparent to those of ordinary skill in the art that variations in the preferred embodiments can be prepared and used and that the invention can be practiced otherwise than as specifically described herein. The present invention is intended to include such variations and alternative practices. Accordingly, this invention includes all modifications encompassed within the spirit and scope of the invention as defined by the following claims.

1. A method of preventing or inhibiting the production of viral DNA in a cell, said method comprising
introducing into said cell, before said cell is infected with a virus that produces viral DNA as part of the virus's life cycle, a conditionally replicating viral vector that integrates into said cell's genome,
wherein said viral vector does not contain a genetic antiviral agent payload that inhibits said virus upon expression.

2. The method of claim 1 wherein said virus is a wildtype virus.

3. The method of claim 2 wherein said virus is HIV-1.

4. The method of claim 3 wherein said cell is a CD4+ cell.

5. The method of claim 1 wherein said cell is a hematopoietic stem cell.

6. The method of claim 1 wherein said cell is a CD4+ cell.

7. The method of claim 1 wherein said vector integrates into the cell's genome by the same process as that used by said virus.

8. The method of claim 1 wherein said conditionally replicating viral vector is introduced at a multiplicity of infection from about 2 to about 50.

9. The method of claim 1 wherein said cell is a primary cell.

10. The method of claim 9 further comprising introducing said cell into a subject.

11. The method of claim 10 wherein said subject is human.

12. The method of claim 11 further comprising the administration of an antiviral agent to said subject.

13. The method of claim 9 wherein said cell is a human cell.

14. The method of claim 1 wherein said conditionally replicating viral vector is derived from an HIV virus.

15. The method of claim 14 wherein said HIV virus is HIV-1.

16. The method of claim 1 wherein said conditionally replicating vector is a chimeric vector.

17. The method of claim 16 wherein said conditionally replicating vector comprises HIV-1 and HIV-2 elements.

18. The method of claim 1 wherein said conditionally replicating vector is pseudotyped before introducing said vector into said cell.

19. The method of claim 18 wherein said conditionally replicating vector is pseudotyped with the vesicular stomatis virus envelope protein VSV-G, the RD114 envelope protein, the Rabies Virus envelope protein, the Gibbon Ape Leukemia Virus envelope protein, or a chimeric envelope protein.

20. The method of claim 5 wherein said conditionally replicating vector expresses a variant of the O⁶-methylguanine-DNA-methyltransferase (MGMT) that is resistant to O⁶-benzylguanine (BG) mediated inactivation and thus protects said cells against alkylating agents.
